(19) United States
(12) Patent Application Publication
(10) Pub. No.: US 2026/0009024 A1
(43) Pub. Date: Jan. 8, 2026

(54) NON-INVASIVE MONITORING OF GENOMIC ALTERATIONS INDUCED BY GENE-EDITING THERAPIES

(71) Applicant: GUARDANT HEALTH, INC., Palo Alto, CA (US)

(72) Inventor: Andrew KENNEDY, San Diego, CA (US)

(21) Appl. No.: 19/282,925

(22) Filed: Jul. 28, 2025

Related U.S. Application Data
(63) Continuation of application No. PCT/US2024/013554, filed on Jan. 30, 2024.
(60) Provisional application No. 63/482,432, filed on Jan. 31, 2023.

(51) Int. Cl. C12N 15/10; C12Q 1/6883

(52) U.S. Cl. CPC C12N 15/1093; C12Q 1/6883

(57) ABSTRACT

Non-invasive, post-gene editing methods for determining the reduced efficacy of a genome-editing drug and/or for detecting diseases induced by a genome-editing drug.

### CROSS-REFERENCE TO RELATED APPLICATIONS

This application is a continuation application of PCT Application No. PCT/US2024/013554, filed Jan. 30, 2024, which claims the benefit of priority of U.S. Provisional Patent Application No. 63/482,432, filed Jan. 31, 2023, which are incorporated by reference herein in its entirety for all purposes.

### INTRODUCTION AND SUMMARY

New genome editing technology has ushered in a new wave of personalized, precision medicines. Few of these have already entered the clinic, with many more in clinical trials, utilizing the technology to directly cure monogenic diseases, as well as cell- and organ-genome engineering for improving immunotherapy and transplantation technology. It is of paramount importance to the field to understand, characterize and minimize off-target genome edits.

Cancer-causing side-effects observed in the previous generation of gene therapy medicines paused/halted clinical adoption and acceptance. With the new wave of CRISPR-based gene-editing there are tremendous technology improvement efforts on-going to minimize off-targets. However, by nature of gene-editing technology mechanism there will arguably will always be some low baseline risk of inducing genetic disease from off-target edits. Current off-target detection methods are limited in sensitivity, unable to assess each cell being edited/administered to a patient. Additionally, each patient's genomic variation can present differences in therapeutic (on-target) efficacy, as well as off-targets, profile, and susceptibility. Furthermore, over time, the efficacy of genome-editing drugs may wane. Therefore, there is a need for improved methods for monitoring effects, including genomic, epigenetic, fragmentomic, and proteomic changes in response to genome editing drugs. The present disclosure aims to meet this need, provide other benefits, or at least provide the public with a useful choice.

### A.

The present disclosure provides methods and systems to monitor genomic, epigenetic, fragmentomic, and/or proteomic changes in response to a gene editing drug to inform treatment follow-ups, such as repeat dosing and side-effect management. Such methods may provide for high sensitivity detection of one or more genetic, epigenetic, fragmentomic, and/or proteomic variants.

In one aspect the disclosure provides a method for determining reduced efficacy of a genome editing drug, comprising: (a) determining a first parameter comprising a quantitative measures of levels of one or more proteins associated with the disease by performing a proteomic assay on molecules obtained or derived from a sample from a subject that has undergone administration of the genome editing drug to treat a disease; (b) determining a second parameter comprising a quantitative measures of editing of one or more predetermined variants associated with the disease by sequencing a sequencing library comprising molecules obtained or derived from a sample from a subject; and (c) determining whether the first and the second parameters are within a predetermined therapeutic threshold, wherein the first and second parameters below the predetermined therapeutic threshold indicate an additional dose of the genome editing drug should be administered to the subject. In an additional aspect, the disclosure provides a method for determining reduced efficacy of a genome editing drug, comprising collecting a sample from a subject, wherein the subject has undergone administration of the genome editing drug to treat a disease; (a) determining a first parameter comprising a quantitative measures of levels of one or more proteins associated with the disease by performing a proteomic assay on molecules obtained or derived from a sample from a subject that has undergone administration of the genome editing drug to treat a disease; (b) determining a second parameter comprising a quantitative measures of editing of one or more predetermined variants associated with the disease by sequencing a sequencing library comprising molecules obtained or derived from a sample from a subject; and (c) determining whether the first and the second parameters are within a predetermined therapeutic threshold, wherein the first and second parameters below the predetermined therapeutic threshold indicate an additional dose of the genome editing drug should be administered to the subject.

In some embodiments the method further comprises determining adverse side effects of the genome editing drug by: (d) sequencing a panel to generate sequencing reads from molecules obtained or derived from a third portion of the sample; and (e) detecting from the sequencing reads the presence or absence of one or more genetic, epigenetic, and/or fragmentomic markers, wherein the presence or absence of one or more of the markers is indicative of the adverse side effect induced by the genome editing drug.

In some embodiments the genome editing drug comprises a CRISPR-Cas system that edits the one or more predetermined variants associated with the disease. In some embodiments the genome editing drug further comprises one or more single guide RNAs (sgRNAs) that direct a Cas protein to the one or more predetermined variants associated with the disease. In some embodiments the Cas proteins comprises Cas9, dCas9, or nCas9. In some embodiments the CRISPR-Cas system introduces a gene knockout, gene insertion, a wildtype allele, or an inactivating mutation.

In some embodiments the sample is a bodily sample comprising blood, plasma, serum, or urine sample.

In some embodiments the proteomic assay comprises an immunoassay, ELISA, HPLC, mass spectrometry, ProtSeq, IHC or immunofluorescence.

In some embodiments the genome editing drug is administered in vivo or ex vivo. In some embodiments the ex vivo genome editing is done in hematopoietic stem cells.

In some embodiments the hematopoietic stem cells comprise a cell-based therapy.

In some embodiments the therapy comprises an immunotherapy.

In some embodiments in the disease is amyloidosis transthyretin-related (ATTR). In some embodiments the one or more variants are within the TTR gene sequence.

In some embodiments the disease is a hemoglobinopathy. In some embodiments the hemoglobinopathy is beta-thalassemia or sickle cell disease.

In some embodiments the adverse side effects is cancer.

In some embodiments at least a subset of the markers in (a) are specific to the genome editing drug. In some embodiments at least a subset of the markers in (a) are specific to the subject based on the subject's genomic profile. In some embodiments the markers in (a) further comprise one or more of a SNV, indels, structural variants, methylation, histone acetylation, TFBS, chromatin positioning, or nucleosome positioning.

In another aspect the disclosure provides a method of screening for a disease induced by a genome editing drug, comprising: (a) sequencing a sequencing panel comprising nucleic acid molecules obtained or derived from the sample to generate sequencing reads; and (b) detecting from the sequencing reads the presence or absence of one or more genetic, epigenetic, and/or fragmentomic markers, wherein the presence or absence of one or more of the markers is indicative of the disease induced by the genome editing drug.

In some embodiments the nucleic acid molecules are DNA. In some embodiments the DNA is cell-free DNA.

In some embodiments copies of the cell-free DNA molecules are generated prior to sequencing.

In some embodiments one or more adapters comprising barcodes are attached to the nucleic acid molecules prior to sequencing. In some embodiments the adapters are randomly attached to both ends of the nucleic acid molecules. In some embodiments the nucleic acid molecules are uniquely barcoded. In some embodiments the nucleic acid molecules are non-uniquely barcoded.

In another aspect the disclosure provides a sequencing panel, comprising: a plurality of target regions, wherein the plurality of target regions comprise a plurality of loci associated with a genetic disease in a subject, wherein the plurality of loci comprise one or more nucleotide targets for a genome editing drug.

In some embodiments the sequencing panel is specific to the subject based on the subject's genomic profile. In some embodiments the panel comprises a plurality of nucleic acid sequences specific to a genome editing drug.

In another aspect the disclosure provides a method for determining therapeutic efficacy and adverse side effects of a genome editing drug, comprising: (a) determining a first parameter comprising a quantitative measures of levels of one or more proteins associated with the disease by performing a proteomic assay on molecules obtained or derived from a first portion of the sample; (b) determining a second parameter comprising a quantitative measures of editing of one or more predetermined variants associated with the disease by sequencing a sequencing library comprising molecules obtained or derived from a second portion of the sample; and (c) determining whether the first and the second parameters are within a predetermined therapeutic threshold, wherein the first and second parameters below the predetermined therapeutic threshold indicate an additional dose of the genome editing drug should be administered to the subject; (d) sequencing a panel to generate sequencing reads from molecules obtained or derived from a third portion of the sample; (c) detecting from the sequencing reads the presence or absence of one or more genetic, epigenetic, and/or fragmentomic markers, wherein the presence or absence of one or more of the markers is indicative of the adverse side effect induced by the genome editing drug.

The disclosure further encompasses the following embodiments. Embodiment 1 is a method for determining reduced efficacy of a genome editing drug, comprising:
(a) determining a first parameter comprising quantitative measures of levels of one or more proteins associated with a disease by performing a proteomic assay on molecules obtained or derived from a sample from a subject that has undergone administration of the genome editing drug to treat the disease;
(b) determining a second parameter comprising quantitative measures of editing of one or more predetermined variants associated with the disease by sequencing a sequencing library comprising molecules obtained or derived from a sample from the subject; and
(c) determining whether the first and the second parameters are within a predetermined therapeutic threshold, wherein the first and second parameters below the predetermined therapeutic threshold indicate an additional dose of the genome editing drug should be administered to the subject.

Embodiment 2 is the method of embodiment 1, wherein the sample of (a) and the sample of (b) are the same sample.

Embodiment 3 is the method of embodiment 1, wherein the sample of (a) and the sample of (b) are different samples.

Embodiment 4 is the method of embodiment 1, wherein an original sample was obtained from the subject, the sample of (a) is a first portion of the original sample, and the sample of (b) is a second portion of the original sample.

Embodiment 5 is the method of any one of the preceding embodiments, wherein the method further comprises determining an adverse side effect of the genome editing drug by:
(d) sequencing a panel to generate sequencing reads from molecules obtained or derived from a sample from the subject; and
(e) detecting from the sequencing reads the presence or absence of one or more genetic, epigenetic, and/or fragmentomic markers, wherein the presence or absence of one or more genetic, epigenetic, and/or fragmentomic markers is indicative of the adverse side effect induced by the genome editing drug.

Embodiment 6 is the method of embodiment 5, wherein the sample of (d) is the same as the sample of (a) and/or the sample of (b).

Embodiment 7 is the method of embodiment 5, wherein the sample of (d) is different from the sample of (a) and/or the sample of (b).

Embodiment 8 is the method of embodiment 5, wherein an original sample was obtained from the subject, the sample of (a) is a first portion of the original sample, the sample of (b) is a second portion of the original sample, and the sample of (d) is a third portion of the original sample; the sample of (a) is a first portion of the original sample, the sample of (d) is a second portion of the original sample, and the sample of (b) is different from the samples of (a) and (d); or the sample of (b) is a first portion of the original sample, the sample of (d) is a second portion of the original sample, and the sample of (a) is different from the samples of (a) and (d).

Embodiment 9 is the method of any one of the preceding embodiments, wherein the genome editing drug comprises a CRISPR-Cas system that edits the one or more predetermined variants associated with the disease.

Embodiment 10 is the method of any one of the preceding embodiments, wherein the genome editing drug comprises one or more single guide RNAs (sgRNAs) that direct a Cas protein to the one or more predetermined variants associated with the disease.

Embodiment 11 is the method of the immediately preceding embodiment, wherein the Cas protein comprises Cas9, dCas9, or nCas9.

Embodiment 12 is the method of embodiment 9, wherein the CRISPR-Cas system introduces a gene knockout, gene insertion, a wildtype allele, or an inactivating mutation.

Embodiment 13 is the method of any one of the preceding embodiments, wherein the sample is a bodily sample comprising a blood, plasma, serum, or urine sample.

Embodiment 14 is the method of any one of the preceding embodiments, wherein the proteomic assay comprises an immunoassay, ELISA, HPLC, mass spectrometry, ProtSeq, IHC or immunofluorescence.

Embodiment 15 is the method of any one of the preceding embodiments, wherein the genome editing drug is administered in vivo or ex vivo.

Embodiment 16 is the method of the immediately preceding embodiment, wherein the ex vivo administration of the genome editing drug is to a hematopoietic stem cell.

Embodiment 17 is the method of the immediately preceding embodiment, wherein the hematopoietic stem cell comprises a cell-based therapy.

Embodiment 18 is the method of the immediately preceding embodiment, wherein the cell-based therapy comprises an immunotherapy.

Embodiment 19 is the method of any one of the preceding embodiments, wherein the disease is amyloidosis transthyretin-related (ATTR).

Embodiment 20 is the method of any one of the preceding embodiments, wherein the one or more predetermined variants are within the TTR gene sequence.

Embodiment 21 is the method of any one of the preceding embodiments, wherein the disease is a hemoglobinopathy.

Embodiment 22 is the method of the immediately preceding embodiment, wherein the hemoglobinopathy is beta-thalassemia or sickle cell disease.

Embodiment 23 is the method of any one of embodiments 5-8, wherein the adverse side effect is cancer.

Embodiment 24 is the method of any one of embodiments 5-8, wherein at least a subset of the genetic, epigenetic, and/or fragmentomic markers in (e) are specific to the genome editing drug.

Embodiment 25 is the method of any one of embodiments 5-8, wherein at least a subset of the genetic, epigenetic, and/or fragmentomic markers in (e) are specific to the subject based on a genomic profile of the subject.

Embodiment 26 is the method of any one of embodiments 5-8, wherein the genetic, epigenetic, and/or fragmentomic markers in (e) further comprise one or more of a single nucleotide variant (SNV), insertion-deletion (indel), structural variant, methylation, histone acetylation, transcription factor binding site (TFBS), chromatin positioning, or nucleosome positioning.

Embodiment 27 is a method of screening for a disease induced by a genome editing drug, comprising:
(a) sequencing a sequencing panel comprising nucleic acid molecules obtained or derived from a sample from a subject that has undergone administration of the genome editing drug to generate sequencing reads; and
(b) detecting from the sequencing reads the presence or absence of one or more epigenetic and/or fragmentomic markers, wherein the presence or absence of one or more of the markers is indicative of the disease induced by the genome editing drug.

Embodiment 28 is a method of screening for a disease induced by a genome editing drug and determining reduced efficacy of the genome editing drug, comprising:
(a) sequencing a sequencing panel comprising nucleic acid molecules obtained or derived from a sample from a subject that has undergone administration of the genome editing drug to generate sequencing reads;
(b) determining a parameter comprising quantitative measures of editing of one or more predetermined variants associated with the disease by sequencing a sequencing library comprising molecules obtained or derived from a sample from the subject; and
(c) detecting from the sequencing reads the presence or absence of one or more genetic, epigenetic, and/or fragmentomic markers, wherein the presence or absence of one or more of the markers is indicative of the disease induced by the genome editing drug.

Embodiment 29 is the method of embodiment 28, wherein the sequencing library and the sequencing panel are present in a single composition and/or are sequenced in a single sequencing cell.

Embodiment 30 is the method of embodiment 28, wherein the sequencing library and the sequencing panel are present in separate compositions and/or are sequenced separately.

Embodiment 31 is the method of embodiment 28 or 29, wherein the sample of (a) and the sample of (b) are the same sample.

Embodiment 32 is the method of embodiment 28 or 30, wherein the sample of (a) and the sample of (b) are different samples.

Embodiment 33 is the method of embodiment 28 or 30, wherein an original sample was obtained from the subject, the sample of (a) is a first portion of the original sample, and the sample of (b) is a second portion of the original sample.

Embodiment 34 is the method of any one of the embodiments 28-33, wherein the genome editing drug comprises a CRISPR-Cas system that edits the one or more predetermined variants associated with the disease.

Embodiment 35 is the method of any one of the embodiments 28-33, wherein the genome editing drug comprises one or more sgRNAs that direct a Cas protein to the one or more predetermined variants associated with the disease.

Embodiment 36 is the method of the immediately preceding embodiment, wherein the Cas protein comprises Cas9, dCas9, or nCas9.

Embodiment 37 is the method of embodiment 34, wherein the CRISPR-Cas system introduces a gene knockout, gene insertion, a wildtype allele, or an inactivating mutation.

Embodiment 38 is the method of any one of the embodiments 27-37, wherein the sample is a bodily sample comprising a blood, plasma, serum, or urine sample.

Embodiment 39 is the method of any one of the embodiments 27-37, wherein the genome editing drug is administered in vivo or ex vivo.

Embodiment 40 is the method of the immediately preceding embodiment, wherein the ex vivo administration of the genome editing drug is to a hematopoietic stem cell.

Embodiment 41 is the method of the immediately preceding embodiment, wherein the hematopoietic stem cell comprises a cell-based therapy.

Embodiment 42 is the method of the immediately preceding embodiment, wherein the cell-based therapy comprises an immunotherapy.

Embodiment 43 is the method of any one of the embodiments 27-42, wherein the disease is amyloidosis transthyretin-related (ATTR).

Embodiment 44 is the method of any one of the embodiments 28-43, wherein the one or more predetermined variants are within the TTR gene sequence.

Embodiment 45 is the method of any one of embodiments 27-42 or 44, wherein the disease is a hemoglobinopathy.

Embodiment 46 is the method of the immediately preceding embodiment, wherein the hemoglobinopathy is beta-thalassemia or sickle cell disease.

Embodiment 47 is the method of any one of embodiments 27-46, wherein at least a subset of the genetic, epigenetic, and/or fragmentomic markers are specific to the genome editing drug.

Embodiment 48 is the method of any one of embodiments 27-47, wherein at least a subset of the genetic, epigenetic, and/or fragmentomic markers are specific to the subject based on a genomic profile of the subject.

Embodiment 49 is the method of any one of embodiments 27-48, wherein the genetic, epigenetic, and/or fragmentomic markers in further comprise one or more of a single nucleotide variant (SNV), insertion-deletion (indel), structural variant, methylation, histone acetylation, transcription factor binding site (TFBS), chromatin positioning, or nucleosome positioning.

Embodiment 50 is the method of any one of embodiments 27-49, wherein the nucleic acid molecules are DNA.

Embodiment 51 is the method of the immediately preceding embodiment, wherein the DNA is cell-free DNA.

Embodiment 52 is the method of the immediately preceding embodiment, further comprising generating copies of the cell-free DNA molecules prior to sequencing.

Embodiment 53 is the method of any one of embodiments 27-49, further comprising generating copies of the cell-free DNA molecules prior to sequencing.

Embodiment 54 is the method of any one of embodiments 27-49, further comprising attaching one or more adapters comprising barcodes to the nucleic acid molecules prior to sequencing.

Embodiment 55 is the method of the immediately preceding embodiment, wherein the one or more adapters are randomly attached to both ends of the nucleic acid molecules.

Embodiment 56 is the method of any one of embodiments 27-55, wherein the nucleic acid molecules are uniquely barcoded.

Embodiment 57 is the method of any one of embodiments 27-55, wherein the nucleic acid molecules are non-uniquely barcoded.

Embodiment 58 is a method of screening for a disease induced by a genome editing drug and determining reduced efficacy of the genome editing drug, comprising:
(a) sequencing a sequencing panel comprising nucleic acid molecules obtained or derived from a sample from a subject that has undergone administration of the genome editing drug to generate sequencing reads;
(b) determining a parameter comprising quantitative measures of levels of one or more proteins associated with the disease by performing a proteomic assay on molecules obtained or derived from a sample from the subject; and
(c) determining whether the first and the second parameters are within a predetermined therapeutic threshold, wherein the first and second parameters below the predetermined therapeutic threshold indicate an additional dose of the genome editing drug should be administered to the subject;
(d) detecting from the sequencing reads the presence or absence of one or more genetic, epigenetic, and/or fragmentomic markers, wherein the presence or absence of one or more of the markers is indicative of the disease induced by the genome editing drug.

Embodiment 59 is the method of embodiment 58, wherein the sample of (a) and the sample of (b) are the same sample.

Embodiment 60 is the method of embodiment 58, wherein the sample of (a) and the sample of (b) are different samples.

Embodiment 61 is the method of embodiment 58, wherein an original sample was obtained from the subject, the sample of (a) is a first portion of the original sample, and the sample of (b) is a second portion of the original sample.

Embodiment 62 is a method for determining therapeutic efficacy and adverse side effects of a genome editing drug, comprising:
(a) determining a first parameter comprising quantitative measures of levels of one or more proteins associated with the disease by performing a proteomic assay on molecules obtained or derived from a sample from a subject that has undergone administration of the genome editing drug;
(b) determining a second parameter comprising quantitative measures of editing of one or more predetermined variants associated with the disease by sequencing a sequencing library comprising molecules obtained or derived from a sample from the subject; and
(c) determining whether the first and the second parameters are within a predetermined therapeutic threshold, wherein the first and second parameters below the predetermined therapeutic threshold indicate an additional dose of the genome editing drug should be administered to the subject;
(d) sequencing a panel to generate sequencing reads from molecules obtained or derived from a third portion of the sample; and
(e) detecting from the sequencing reads the presence or absence of one or more genetic, epigenetic, and/or fragmentomic markers, wherein the presence or absence of one or more of the markers is indicative of the adverse side effect induced by the genome editing drug.

Embodiment 63 is the method of embodiment 62, wherein the sample of (a) and the sample of (b) are the same sample.

Embodiment 64 is the method of embodiment 62, wherein the sample of (a) and the sample of (b) are different samples.

Embodiment 65 is the method of embodiment 62, wherein an original sample was obtained from the subject, the sample of (a) is a first portion of the original sample, and the sample of (b) is a second portion of the original sample.

Embodiment 66 is the method of embodiment 58, wherein the nucleic acid molecules are DNA.

Embodiment 67 is the method of the immediately preceding embodiment, wherein the DNA is cell-free DNA.

Embodiment 68 is the method of the immediately preceding embodiment, further comprising generating copies of the cell-free DNA molecules prior to sequencing.

Embodiment 69 is the method of any one of embodiments 58-61 or 66-68, further comprising generating copies of nucleic acid molecules prior to sequencing.

Embodiment 70 is the method of any one of embodiments 58-61 or 66-69, further comprising attaching one or more adapters comprising barcodes to the nucleic acid molecules prior to sequencing.

Embodiment 71 is the method of the immediately preceding embodiment, wherein the adapters are randomly attached to both ends of the nucleic acid molecules.

Embodiment 72 is the method of any one of embodiments 58-61 or 66-71, wherein the nucleic acid molecules are uniquely barcoded.

Embodiment 73 is the method of any one of embodiments 58-61 or 66-71, wherein the nucleic acid molecules are non-uniquely barcoded.

Embodiment 74 is the method of any one of embodiments 62-65, wherein the genome editing drug comprises a CRISPR-Cas system that edits the one or more predetermined variants associated with the disease.

Embodiment 75 is the method of any one of embodiments 62-65 or 74, wherein the genome editing drug comprises one or more single guide RNAs (sgRNAs) that direct a Cas protein to the one or more predetermined variants associated with the disease.

Embodiment 76 is the method of the immediately preceding embodiment, wherein the Cas protein comprises Cas9, dCas9, or nCas9.

Embodiment 77 is the method of embodiment 74, wherein the CRISPR-Cas system introduces a gene knockout, gene insertion, a wildtype allele, or an inactivating mutation.

Embodiment 78 is the method of any one of embodiments 58-77, wherein the sample is a bodily sample comprising a blood, plasma, serum, or urine sample.

Embodiment 79 is the method of any one of embodiments 62-65 or 74-78, wherein the proteomic assay comprises an immunoassay, ELISA, HPLC, mass spectrometry, ProtSeq, IHC or immunofluorescence.

Embodiment 80 is the method of any one of embodiments 58-79, wherein the genome editing drug is administered in vivo or ex vivo.

Embodiment 81 is the method of the immediately preceding embodiment, wherein the ex vivo administration of the genome editing drug is to a hematopoietic stem cell.

Embodiment 82 is the method of the immediately preceding embodiment, wherein the hematopoietic stem cell comprises a cell-based therapy.

Embodiment 83 is the method of the immediately preceding embodiment, wherein the cell-based therapy comprises an immunotherapy.

Embodiment 84 is the method of any one of embodiments 58-83, wherein the disease is amyloidosis transthyretin-related (ATTR).

Embodiment 85 is the method of any one of embodiments 62-65 or 74-77, wherein the one or more predetermined variants are within the TTR gene sequence.

Embodiment 86 is the method of any one of embodiments 58-83 or 85, wherein the disease is a hemoglobinopathy.

Embodiment 87 is the method of the immediately preceding embodiment, wherein the hemoglobinopathy is beta-thalassemia or sickle cell disease.

Embodiment 88 is the method of any one of embodiments 62-65, 74-79, or 85, wherein the adverse side effect is cancer.

Embodiment 89 is the method of any one of embodiments 58-88, wherein at least a subset of the genetic, epigenetic, and/or fragmentomic markers are specific to the genome editing drug.

Embodiment 90 is the method of any one of embodiments 58-89, wherein at least a subset of the genetic, epigenetic, and/or fragmentomic markers are specific to the subject based on a genomic profile of the subject.

Embodiment 91 is the method of any one of embodiments 58-90, wherein the genetic, epigenetic, and/or fragmentomic markers further comprise one or more of a single nucleotide variant (SNV), insertion-deletion (indel), structural variant, methylation, histone acetylation, transcription factor binding site (TFBS), chromatin positioning, or nucleosome positioning.

Embodiment 92 is a method of preparing a panel of hybridization probes, comprising: (a) sequencing nucleic acid molecules obtained or derived from a sample from a subject that has undergone administration of a genome editing drug, thereby obtaining sequencing reads; (b) identifying a plurality of genomic locations with alterations using the sequencing reads; (c) preparing the panel of hybridization probes, wherein the panel comprises hybridization probes specific for at least a portion of the plurality of genomic locations with alterations.

Embodiment 93 is the method of the immediately preceding embodiment, wherein the hybridization probes are labeled with a binding partner, optionally wherein the binding partner is biotin.

Embodiment 94 is a method of preparing a sequencing library, comprising:
(a) sequencing nucleic acid molecules obtained or derived from a sample from a subject that has undergone administration of a genome editing drug, thereby obtaining sequencing reads;
(b) identifying a plurality of genomic locations with alterations using the sequencing reads;
(c) enriching a second sample from the subject for a plurality of target regions, wherein the target regions comprise at least a portion of the genomic locations identified in (b), thereby providing enriched target regions;
(d) preparing a sequencing library comprising at least a portion of the enriched target regions.

Embodiment 95 is the method of the immediately preceding embodiment, wherein enriching the second sample comprises targeted amplification of the plurality of target regions.

Embodiment 96 is the method of embodiment 94, wherein enriching the second sample comprises capturing the plurality of target regions with a plurality of hybridization probes.

Embodiment 97 is the method of any one of embodiments 94-96, wherein preparing the sequencing library comprises adding adapters to at least a portion of the enriched target regions.

Embodiment 98 is the method of the immediately preceding embodiment, wherein the adapters comprise barcodes.

Embodiment 99 is the method of any one of embodiments 94-96, wherein preparing the sequencing library comprises amplifying the enriched target regions.

Embodiment 100 is the method of any one of embodiments 92-99, wherein the alterations comprise on-target genetic alterations.

Embodiment 101 is the method of any one of embodiments 92-99, wherein the alterations comprise off-target genetic alterations.

Embodiment 102 is the method of any one of embodiments 92-99, wherein the alterations comprise epigenetic alterations.

Embodiment 103 is the method of any one of embodiments 92-99, wherein the alterations comprise fragmentomic alterations.

Embodiment 104 is the method of any one of embodiments 92-99, wherein the alterations are relative to a wild-type reference genome.

Embodiment 105 is the method of any one of embodiments 92-99, wherein alterations are relative to the genome of the subject before administration of the genome editing drug.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE FIGURES

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows current in vivo editing workflows.

FIG. 2 shows an in vivo editing workflow for monitoring informed secondary dose of a therapeutic.

FIG. 3 shows an in vivo editing workflow for early detection of cancer (initiated/mediated by genome-editing off-target).

FIG. 4 is a schematic diagram of an example of a system suitable for use with some embodiments of the disclosure.

### DETAILED DESCRIPTION

Reference will now be made in detail to certain embodiments of the disclosure. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

All patents, patent applications, websites, other publications or documents and the like cited herein whether supra or infra, are expressly incorporated by reference in their entirety for all purposes to the same extent as if each individual item were specifically and individually indicated to be so incorporated by reference. If different versions of a publication, website or the like are published at different times, the version most recently published at the effective filing date of the application is meant, unless otherwise indicated.

### I. Definitions

As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

As used herein, “partitioning” refers to physically separating, sorting, and/or fractionating a mixture of nucleic acid molecules in a sample into a plurality of subsamples or subpopulations of nucleic acids based on a characteristic of the nucleic acid molecules. A sample or population may be partitioned into one or more partitioned subsamples or subpopulations based on a characteristic that is indicative of a genetic or epigenetic change or a disease state. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. Stated differently, partitioning may include physically partitioning nucleic acid molecules based on the presence or absence of one or more methylated nucleobases. In some embodiments, the methods and systems used for partitioning may be found in PCT Patent Application No. PCT/US2017/068329, which is hereby incorporated by reference in its entirety.

As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets-one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partition, or residual partitioned set). In another example, the nucleic acid molecules can be partitioned based on the number of methylated nucleotides-one partitioned set can have nucleic acid molecules with nine methylated nucleotides, and another partitioned set can have unmethylated nucleic acid molecules (zero methylated nucleotides).

As used herein, “sample” means anything capable of being analyzed by the methods and/or systems disclosed herein.

As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, long-read sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Examples of commercially available instruments for performing next-generation sequencing include, but are not limited to, NextSeq, HiSeq, NovaSeq, MiSeq, Ion PGM and Ion GeneStudio S5.

As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

As used herein, “base pairing specificity” refers to the standard DNA base (A, C, G, or T) for which a given base most preferentially pairs. Thus, for example, unmethylated cytosine, 5mC and 5hmC have the same base pairing specificity (i.e., specificity for G) whereas uracil and cytosine have different base pairing specificity because uracil has base pairing specificity for A while cytosine has base pairing specificity for G. The ability of uracil to form a wobble pair with G, for example, is irrelevant because uracil nonetheless most preferentially pairs with A among the four standard DNA bases.

“Enriching” or “Capturing” one or more target nucleic acids or one or more nucleic acids comprising at least one target region refers to preferentially isolating or separating the one or more target nucleic acids or one or more nucleic acids comprising at least one target region from non-target nucleic acids or from nucleic acids that do not comprise at least one target region, e.g., through the use of targeted sequence capture.

A nucleic acid is “produced by a tumor” or ctDNA or circulating tumor DNA, if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence)). 5-methylcytosine (5mC) refers to a cytosine with a methyl group added to the 5C position of the cytosine. Derivatives of 5mC include, but are not limited to, 5-hydroxymethylcytosine (5hmC), 5-formylcytosine (5fC), and 5-caryboxylcytosine (5caC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The “methylation profile of nucleic acids” means the position and identity of the nucleoside and the methylation status of the nucleoside within a nucleic acid (e.g., DNA) sequence. As described above, different methods of conversion and partitioning followed by sequencing can detect unmethylated C, 5mC and 5hmC profiled. The methylation profile of cytosines can be identified according to the specific partitioning and conversion procedures as described above.

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, “fragment” refers to a biological component, such as a nucleic acid molecule (such as DNA or RNA) that has been broken or separated from one or more other pieces. Fragmentation, such as DNA fragmentation, can occur spontaneously (as in cfDNA fragments, which may be obtained from blood samples) or can be induced intentionally, such as using standard laboratory procedures, such as described herein. DNA fragmentation can be performed, for example, to prepare DNA (such as genomic DNA and/or DNA isolated from a sample comprising cells) for sequencing. With some samples, such as cfDNA samples, artificial fragmentation may be unnecessary.

As used herein, a “fragmentation characteristic” refers to any feature relating to the endpoints, midpoint, size, presence, absence, and/or amount of DNA fragments as isolated from a subject, such as DNA fragments having a midpoint or one or both endpoints at a particular genomic position or within a particular range of positions, and/or having a length of a particular value or in a particular range.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

An “enriched set” or “captured set” of nucleic acids or “enriched” or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “cell type” is a set of cells having a shared characteristic. For example, cell types can include cells of different origins, differentiation types, different activation types, or any combination of different origins, different differentiation types, and different activation types. Indeed, differentiation status and activation status can overlap and often change together in a given cell, such as an immune cell or a cancer cell. For example, activation of an immune cell may induce differentiation of the cell. In some embodiments, cell types may be distinguished based on characteristics such as one or more cell surface markers, a genetic signature (such as expression (or expression level) of a particular gene or set of genes), and/or an epigenetic signature, such as regions of DNA hypermethylation or hypomethylation.

As used herein, a “cell cluster” or “cluster” is a plurality of related cell types, e.g., immune cell types, tissue-specific cell types, and/or cancer cell types. In some embodiments, the cell types within a cluster have similar DNA methylation profiles, e.g., in a plurality of hypermethylation variable target regions and/or hypomethylation variable target regions.

A “converted nucleobase” is a nucleobase having an altered base pairing specificity, wherein the original base pairing specificity of the nucleobase was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. As used herein, a “converted sample” is a sample comprising DNA comprising at least one converted nucleobase.

As used herein, a “combination” of steps or other elements refers to the performance or presence of two or more of the steps or elements in a method or product; elements, where appropriate, may be either together in a single composition, apparatus, or the like, or in proximity, e.g., in separate containers or compartments within a larger container, such as a multiwell plate, tube rack, refrigerator, freezer, incubator, water bath, ice bucket, machine, or other form of storage. A combination, combinations, or combination thereof refers to any and all permutations and combinations of the listed terms preceding the term “combination.” For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Specifically binds” in the context of a primer, a probe, or other oligonucleotide and a target sequence (e.g., a nucleic acid comprising a sequence that is partially or completely complementary to the primer, probe, or other oligonucleotide) means that under appropriate hybridization conditions, the primer, probe, or other oligonucleotide hybridizes to its target sequence, or replicates thereof, to form a stable hybrid, while at the same time formation of stable non-target hybrids is minimized. Thus, a primer, probe, or other oligonucleotide hybridizes to a target sequence or replicate thereof to a sufficiently greater extent than to a non-target sequence, to ultimately enable enrichment or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

A “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity). A “target region set” can comprise regions that share at least one common feature. In some embodiments, a target region set is identified by the at least one common feature. For example, a hypermethylation variable target region set comprises regions of DNA that are hypermethylated.

“Sequence-variable target regions” refer to target regions that may exhibit changes in sequence such as nucleotide substitutions (i.e., single nucleotide variations), insertions, deletions, or gene fusions, or transpositions in neoplastic cells (e.g., tumor cells and cancer cells) relative to normal cells. A “sequence-variable target region set” refers to a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, 2, or 1 nucleotides.

“Epigenetic target regions” refers to target regions that may show sequence-independent differences in different cell or tissue types (e.g., different types of immune cells) or in abnormal cells, such as neoplastic cells (e.g., tumor cells and cancer cells), relative to normal cells; or that may show sequence-independent differences (i.e., in which there is no change to the nucleotide sequence, e.g., differences in methylation, nucleosome distribution, or other epigenetic features) in DNA, e.g., from different cell types or from subjects having cancer relative to DNA from healthy subjects. Examples of sequence-independent changes include, but are not limited to, changes in methylation (increases or decreases), nucleosome distribution, fragmentation patterns, CCCTC-binding factor (“CTCF”) binding, transcription start sites (e.g., with respect to any one or more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An “epigenetic target region set” is a set of epigenetic target regions.

As used herein, a “differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one immune cell type, relative to the degree of methylation in the same region of DNA from at least one other cell or tissue type, such as other immune cell types, or from the same cell or tissue type from a healthy subject. In some embodiments, a differentially methylated region has a detectably lower degree of methylation (e.g., a hypomethylated region) in at least one cell or tissue type, such as at least one immune cell type, relative to the degree of methylation in the same region of DNA from at least one other cell or tissue type, such as other immune cell types, or from the same cell or tissue type from a healthy subject.

A nucleic acid is “produced by a tumor” if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “hypermethylation” refers to an increased level or degree of methylation of nucleic acid molecule(s) relative to the other nucleic acid molecules comprising the same genetic information within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules comprising the same genetic information within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. Suitable agents include agents that recognize modified nucleotides in double-stranded DNA, single-stranded DNA, and both double-stranded and single-stranded DNA.

The term “epigenetic status” refers to a certain level or extent of a sequence-independent variable that may be present in a DNA sequence. In some embodiments, the epigenetic status of a DNA sequence refers to the extent or level of methylation, nucleosome distribution, cfDNA fragmentation pattern, CCCTC-binding factor (“CTCF”) binding, transcription start site, or regulatory protein binding region of the sequence. Epigenetic statuses thus include, but are not limited to, hypermethylation, hypomethylation, and the presence of absence of CTCF binding sites or transcription start sites. The epigenetic status of a sequence may be a “reference epigenetic status” that can be used for comparison to the epigenetic status of the corresponding sequence in other DNA molecules. An example of a reference epigenetic status is a status that is prevalent in samples obtained from healthy subjects and is not associated with cancer.

As used herein, “methylation status” refers to the presence or absence of a methyl group on a DNA nucleobase (e.g., cytosine) at a particular genomic position in a nucleic acid, the degree of methylation of a nucleic acid (e.g., high, low, intermediate, or unmethylated), or the number of nucleotides methylated in a particular nucleic acid molecule. A nucleic acid “in methylated form” means that it comprises a sequence containing a methylated DNA nucleobase, e.g., a methylated cytosine in a CpG dinucleotide.

As used herein, “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

As used herein, “methylation sensitive restriction enzyme” or “MSRE” refers to a methylation sensitive nuclease that is a restriction enzyme. An MSRE is sensitive to the methylation status of the DNA (e.g., cytosine methylation), i.e., the presence or absence of methyl group in a nucleotide base in its recognition sequence alters the rate at which the enzyme cleaves the DNA. In some embodiments, the methylation sensitive restriction enzymes do not cleave the DNA if a particular nucleotide base is methylated at the recognition sequence. For example, HpaII is a methylation sensitive restriction enzyme with a recognition sequence “CCGG” and it does not cleave DNA if the second cytosine in the recognition sequence is methylated.

As used herein, “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

As used herein, “methylation-dependent restriction enzyme” or “MDRE” refers to a methylation dependent nuclease that is a restriction enzyme. An MDRE is dependent on methylation of the DNA (e.g., cytosine methylation) i.e., the presence or absence of methyl group in a nucleotide base alters the rate at which the enzyme cleaves the DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR (N9)” and it does not cleave DNA if the absence of the methylated cytosine (mC) in the recognition sequence.

As used herein, “digestion efficiency” or “cutting efficiency” refers to the efficiency of restriction enzyme digestion. The digestion efficiency can be calculated based on the number of control molecules observed upon digesting with restriction enzyme and number of control molecules observed in the absence of restriction enzyme digestion. The MSRE digestion efficiency can be calculated by: Efficiency=1−(number of negative control molecules_{[MSRE]}/number of negative control molecules_{[Mock]}). The MDRE digestion efficiency can be calculated by: Efficiency=1−(number of positive control molecules_{[MDRE]}/number of positive control molecules_{[Mock]}).

“Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

A “T1392S mutation” as used herein refers to a substitution in a TET2 enzyme of the threonine present at position 1372 of the full-length wild-type human TET2 enzyme with a serine. A TET2 enzyme comprising a T1372S mutation may, but does not necessarily, comprise additional differences from the wild-type human enzyme sequence. Position 1372 aligns to position 258 and 248, respectively, of the truncated TET2 sequences disclosed as SEQ ID NOs: 23 and 24 of U.S. Pat. No. 10,961,525. The immediate wild-type sequence context of position 1372 of human TET2 is FSGVTACLD (SEQ ID NO: 13) where the T is at position 1372. Thus, a TET2 enzyme comprising a T1392S mutation may comprise the sequence FSGVSACLD (SEQ ID NO: 14) or optionally a variant of SEQ ID NO: 14 in which at least 5, 6, 7, or 8 positions match SEQ ID NO: 14 including position 5.

“Or” is used in the inclusive sense, i.e., equivalent to “and/or,” unless the context requires otherwise.

### II. Exemplary Methods

### B. Overview

Improvements in genome editing technology has ushered in a new wave of personalized, precision medicines. As gene-editing therapeutics are being adopted into clinical practice there is a perceived need for post-administration non-invasive diagnostics to monitor and detect in vivo therapeutic efficacy (short/long-term) and side effects (immediate/proliferating off-targets) to guide follow-up treatment. With the latter, all such therapies, with mechanism of specifically altering the genome have potential side effects of inducing genetic diseases, such as cancer through off-target alterations.

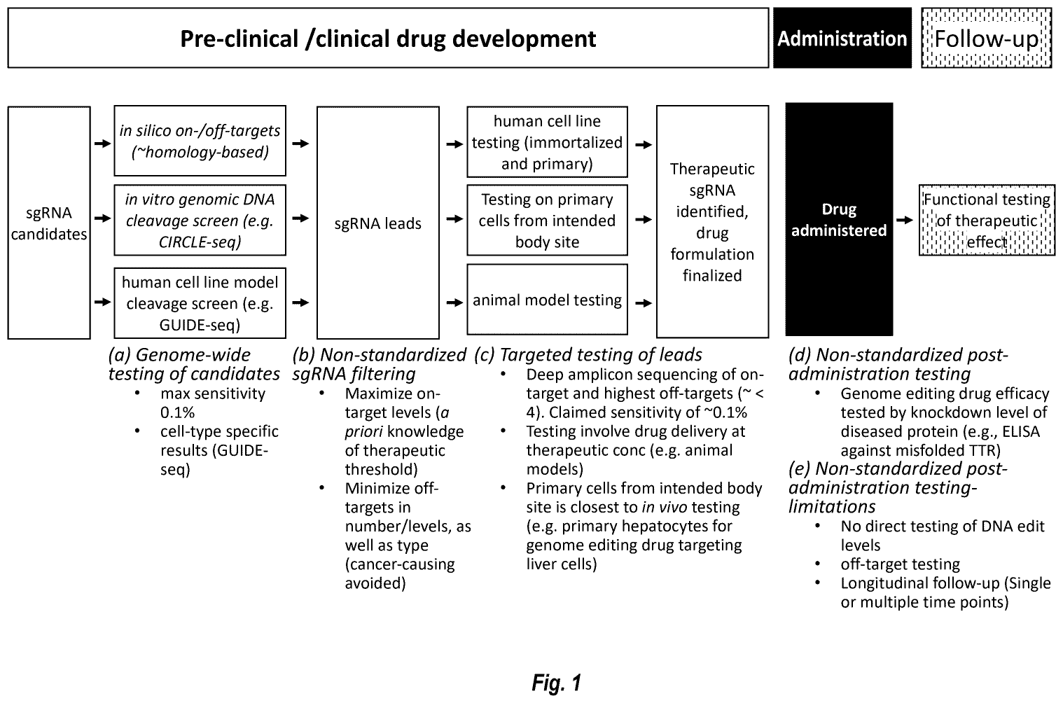

FIG. 1 shows current in vitro biochemical methods (CIRCLE-seq, GUIDE-seq) for finding off-targets, which only offer a maximum sensitivity of ˜0.1%, 4 orders of magnitude higher than what would be needed to assess the single cell off-target editing event in a 100M+ cell dose (FIG. 1; step a). These methods are applied to filter guide RNAs, with maximum on-target levels (a priori knowledge of therapeutic threshold) and minimum off-targets in number/levels, as well as to avoid cancer-causing off-target sites (FIG. 1; step b). In vivo gene editing therapies currently test on-target efficacy and off-targets by proxy, only during drug development and using in silico, in animal, and in vitro cell models (FIG. 1; step c). Following administration of the gene editing drug in vivo, functional testing of therapeutic effect is done using proteomic assays (e.g., NTLA-2001 an ELISA against misfolded TTR is performed to confirm knockdown level of the diseased protein) (FIG. 1; step d). These limited follow-up studies do not address the off-target edits that can occur at different tissues across the body, other than intended tissue site and patients do not benefit from follow-up treatment options, such as secondary dose or alternative treatments (FIG. 1; step e).

An aspect of the present disclosure provides methods for non-invasive, post gene-editing monitoring for therapeutic efficacy and/or early cancer detection (and other genetic diseases) utilizing, in some embodiments, liquid biopsy technology. Off-target gene editing may induce carcinogenesis through somatic alterations of tumor suppressor and/or oncogenes-sensitive liquid biopsy cancer screening technologies incorporating somatic and epigenetic markers can be employed for early detection when there is better chance of successful intervention. A plasma-based cf/ctDNA methods is envisioned to be particularly valuable in all “in vivo editing” applications (editing reagents directly administered to patient's body-target solid tissue, off-target potential body-wide) and transplantation of non-hematological cells/tissues.

A relatively low editing threshold (% of cells edited) is necessary for many therapies, as the corrected cells have a proliferative advantage over diseased cells (e.g., sickle cell disease correction of hematopoietic stem cells ex vivo). Although this is seemingly positive for clinical efficacy, concerns have been raised by multiple researchers who have observed that cells with deficient DNA damage response (e.g., TP53 deficient) are preferentially edited. Cells with deficient/inhibited DNA damage response can arise from spontaneous, background mutations or gene-editing off-targets (in same cell as on-target edits). The proliferative advantage of corrected cells with deficient DNA damage response increases oncogenic risk.

In other or additional embodiments, the long-term efficacy of therapeutic dose may vary from patient to patient. Assessing frequency of edited cells via turnover in cfDNA can be used to evaluate if/when secondary doses will be needed to maintain therapeutic levels of corrected cells. For example, with their NTLA-2001 drug, Intellia has shown post-administration functional testing of therapeutic effect—immunoassay to quantify level of the disease-causing protein (misfolded TTR). While this is valuable, if changes in therapeutic effect occur, follow-up treatment options, such as secondary dose will need to be informed by absolute/changes in levels of genome alterations. If edited DNA remains above known therapeutic threshold, but therapeutic effect has been lost, further investigation of cause and alternative treatment options should be considered-second dose of gene-editing drug is not warranted.

Secondary or additional dose of genome-editing reagents also increase likelihood of off-targets. Yet no drug has shown secondary dosing data. Such data, showing off-target assessment after administration of additional dose(s) may likely be required by regulatory agencies. In either scenario, monitoring changes in edited cell frequency post-administration will be a valuable diagnostic to guide clinical follow-up.

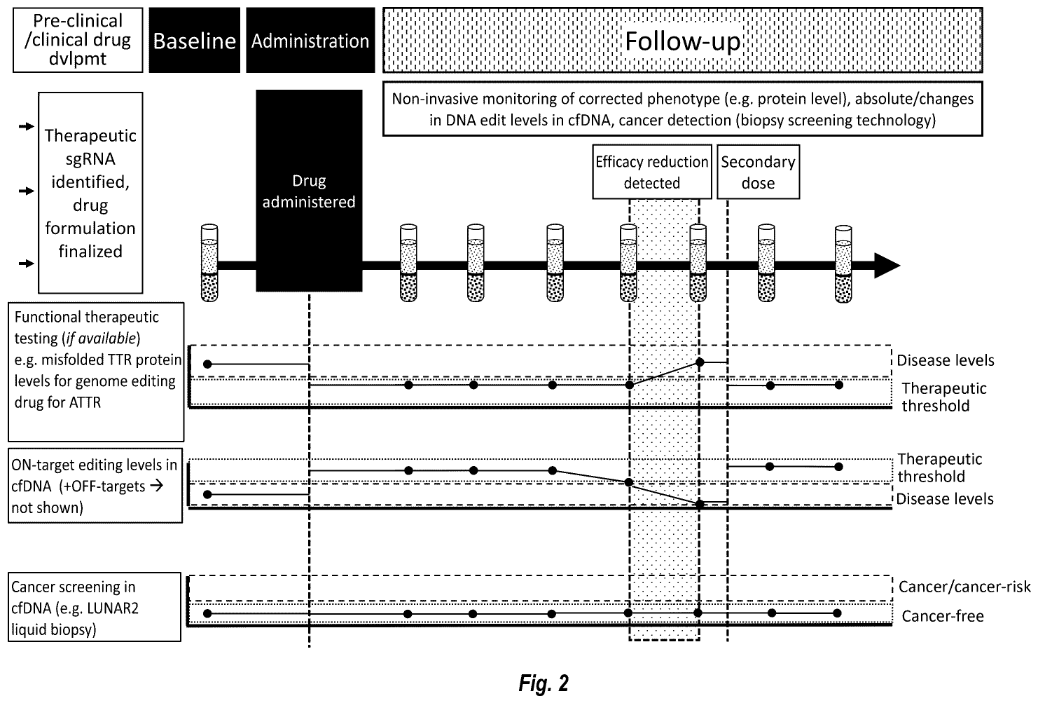

FIG. 2 shows an example of a method for determining the efficacy of an administered therapeutic. Since each patient's genome can present differences in therapeutic (on-target) efficacy, as well as off-targets, profile and susceptibility post-administration quantification of in vivo editing can offer patients follow-up treatment options, such as secondary dose or alternative treatments when a therapeutic effect is not observed and/or is lost. In the embodiment show in FIG. 2, in vivo editing workflow baseline values for protein levels, functional therapeutic tests, on-target editing levels, and markers for disease risk for are established prior to administration of the drug, and a longitudinal follow-up is conducted post-administration. Over multiple time points a patient exhibiting drug efficacy reduction (levels of on-target editing below therapeutic thresholds) can be administered a second dose to restore therapeutic levels of on-target editing.

Gene editing therapeutics can be grouped by where editing event occurs-ex vivo or in vivo. The former is primarily focused on hematologic monogenic diseases, whereby blood cells are removed from patient, gene-edited ex vivo and re-administered to the patient (autologous edited cell therapy/transplantation). Commonly, 100s of millions of cells subjected to editing are transplanted back into body with these processes. Before patient re-administration, the standard practice to assess editing safety is to perform amplicon sequencing of the on-target and a few in silico predicted off-targets. The claimed sensitivity of these methods is ˜0.1%, 4 orders of magnitude higher than what would be needed to assess the single cell off-target editing event in the 100M+ cell dose. Engineering cells by gene-editing for novel cell-based therapies (e.g., immunotherapy) and editing animal organs for safety in human transplantation (xenographs) have same conceptual concerns as direct ex vivo gene-editing therapies.

In vivo gene editing therapies currently test on-target efficacy and off-targets by proxy, only during drug development—in silico, in animal, and cell models. There is no post-administration quantification of in vivo editing. This is of concern for same reasons as above, with added complexity that off-target edits could occur at different body locations, other than intended body site (e.g., liver cells in transthyretin (ATTR) amyloidosis patients). As such, gene-editing off-targets should be assessed globally-cell-free DNA diagnostics are well suited for this, assaying the DNA after cell death from various cell types in the body. This approach also provides a non-invasive tool to monitor therapeutic efficacy-monitoring absolute level and temporal changes of on-target editing in cfDNA.

The present disclosure provides methods valuable as a post-treatment monitoring test for gene editing drugs, such as Intellia Therapeutics' NTLA-2001 therapy for ATTR amyloidosis, a rare and fatal disease that occurs in people born with TTR gene mutations, that produce a misfolded protein. NTLA-2001 is undergoing clinical trials with successful (interim) first-in-human results from an in vivo editing therapy. The therapy delivers CRISPR editing reagents (as RNA) in a lipid nanoparticle to liver cells, and the CRISPR-Cas9 system targets the TTR gene, causing an inactivating mutation that inhibits production of the misfolded protein. Liquid biopsy monitoring post-therapy can be used to assess (1) if therapeutic levels of editing have been achieved, (2) are they being sustained, (3) the frequency of and changes in levels of off-target edits, are any clinically relevant, (4) is there evidence of cancer or cancer/risk resulting from treatment.

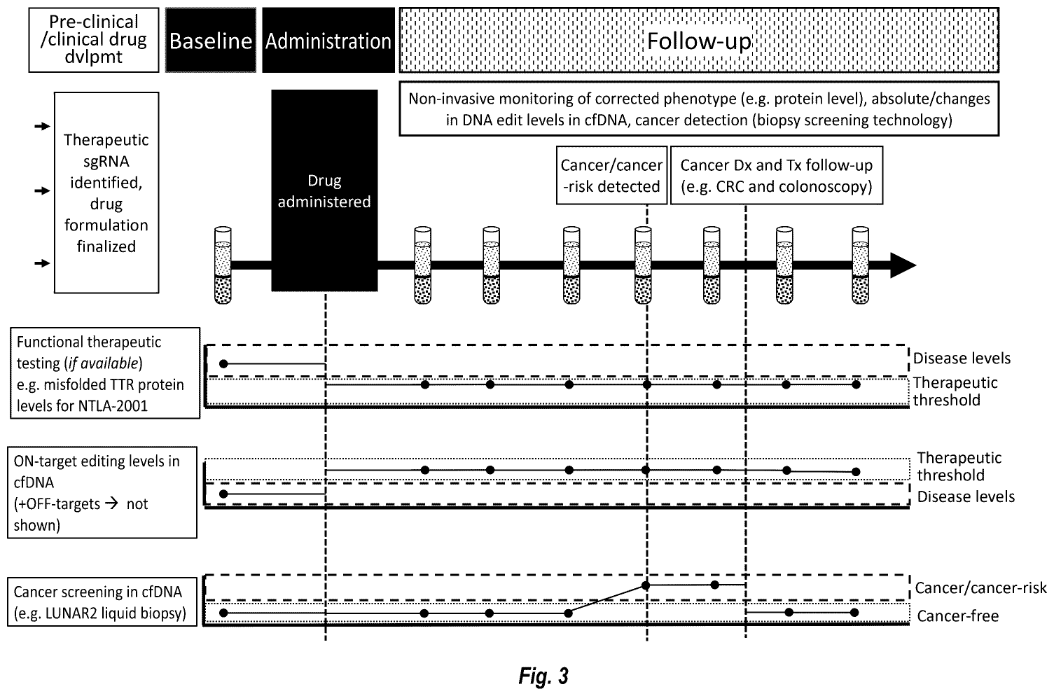

Gene editing therapies, with mechanism of specifically altering the genome have potential side effects of inducing genetic diseases, through off-target alterations. For example, off-target gene editing may induce carcinogenesis through somatic alterations of tumor suppressor and/or oncogenes. Longitudinal monitoring using sensitive liquid biopsy cancer screening technologies incorporating somatic and epigenetic markers can be employed for early detection when there is better chance of successful intervention. In FIG. 3, an embodiment of the present disclosure provides an in vivo editing workflow where baseline values for markers for disease risk are established prior to administration of the drug, and a longitudinal follow-up is conducted post-administration. Over multiple time points a patient exhibiting markers of disease risk (levels of markers above therapeutic thresholds) can undergo standard of care testing to confirm risk (e.g., in CRC a colonoscopy) and appropriate treatment to restore therapeutic levels of markers.

The present disclosure provides methods to develop an assay, such as a liquid biopsy, utilizing appropriate cancer screening test (cancer-specific or multi-cancer test) and incorporate into a targeted sequencing workflow (e.g., hybrid capture panel) relevant on- and off-target regions specific to the gene-editing therapy. Administer this assay to a gene-editing patient at various timepoints post-treatment (optionally pre-treatment) to monitor genetic and epigenetic alterations associated with treatment in cfDNA or tissue.

In some embodiments, a sequencing panel can be developed, customized and/or upgraded-adding content relevant to genome-editing applications. Growth and adoption of genome-editing in the future for clinical, wellness/improvements, bio-terror, others will require regular/more-frequent monitoring of genome-integrity. Adding content and methods to specifically detect genome-editing reagent integration into genome may also be valuable.

In some embodiments, methods and systems are provided to monitor genomic changes in response to a gene editing drug to inform treatment follow-ups, such as repeat dosing and side-effect management. Such methods may provide for high sensitivity detection of one or more genetic variants. Throughout this disclosure, the steps of the disclosed methods are not necessarily performed in the order listed except to the extent that a product or outcome of one step is needed to perform a later step.

In some embodiments, a method for determining reduced efficacy of a genome editing drug is provided, comprising: (a) determining a first parameter comprising quantitative measures of levels of one or more proteins associated with a disease by performing a proteomic assay on molecules obtained or derived from a sample from a subject that has undergone administration of the genome editing drug to treat the disease; (b) determining a second parameter comprising quantitative measures of editing of one or more predetermined variants associated with the disease by sequencing a sequencing library comprising molecules obtained or derived from a sample from the subject; and (c) determining whether the first and the second parameters are within a predetermined therapeutic threshold, wherein the first and second parameters below the predetermined therapeutic threshold indicate an additional dose of the genome editing drug should be administered to the subject. In some embodiments, a method for determining reduced efficacy of a genome editing drug is provided, comprising collecting a sample from a subject, wherein the subject has undergone administration of the genome editing drug to treat a disease; (a) determining a first parameter comprising a quantitative measures of levels of one or more proteins associated with the disease by performing a proteomic assay on molecules obtained or derived from a sample from a subject that has undergone administration of the genome editing drug to treat a disease; (b) determining a second parameter comprising a quantitative measures of editing of one or more predetermined variants associated with the disease by sequencing a sequencing library comprising molecules obtained or derived from a sample from a subject; and (c) determining whether the first and the second parameters are within a predetermined therapeutic threshold, wherein the first and second parameters below the predetermined therapeutic threshold indicate an additional dose of the genome editing drug should be administered to the subject.

In some embodiments, the sample of (a) and the sample of (b) are the same sample. In some embodiments, the sample of (a) and the sample of (b) are different samples. In some embodiments, an original sample can be obtained from the subject, the sample of (a) is a first portion of the original sample, and the sample of (b) is a second portion of the original sample.

In some embodiments, the method for determining reduced efficacy of a genome editing drug further comprises determining adverse side effects of the genome editing drug by: (d) sequencing a panel to generate sequencing reads from molecules obtained or derived from a sample from the subject; and (c) detecting from the sequencing reads the presence or absence of one or more genetic, epigenetic, and/or fragmentomic markers, wherein the presence or absence of one or more genetic, epigenetic, and/or fragmentomic markers is indicative of the adverse side effect induced by the genome editing drug.

In some embodiments, the sample of (d) is the same as the sample of (a) and/or the sample of (b). In some embodiments, the sample of (d) is different from the sample of (a) and/or the sample of (b). In some embodiments, an original sample can be obtained from the subject, the sample of (a) is a first portion of the original sample, the sample of (b) is a second portion of the original sample, and the sample of (d) is a third portion of the original sample; the sample of (a) is a first portion of the original sample, the sample of (d) is a second portion of the original sample, and the sample of (b) is different from the samples of (a) and (d); or the sample of (b) is a first portion of the original sample, the sample of (d) is a second portion of the original sample, and the sample of (a) is different from the samples of (a) and (d).

In some embodiments, a method of screening for a disease induced by a genome editing drug is provided, comprising: (a) sequencing a sequencing panel comprising nucleic acid molecules obtained or derived from a sample from a subject that has undergone administration of the genome editing drug to generate sequencing reads; and (b) detecting from the sequencing reads the presence or absence of one or more epigenetic and/or fragmentomic markers, wherein the presence or absence of one or more of the markers is indicative of the disease induced by the genome editing drug. In some embodiments, the sample of (a) and the sample of (b) are the same sample. In some embodiments, the sample of (a) and the sample of (b) are different samples. In some embodiments, an original sample was obtained from the subject, the sample of (a) is a first portion of the original sample, and the sample of (b) is a second portion of the original sample.

In some embodiments, a method of screening for a disease induced by a genome editing drug and determining reduced efficacy of the genome editing drug is provided, comprising: (a) sequencing a sequencing panel comprising nucleic acid molecules obtained or derived from a sample from a subject that has undergone administration of the genome editing drug to generate sequencing reads; (b) determining a parameter comprising quantitative measures of editing of one or more predetermined variants associated with the disease by sequencing a sequencing library comprising molecules obtained or derived from a sample from the subject; and (c) detecting from the sequencing reads the presence or absence of one or more genetic, epigenetic, and/or fragmentomic markers, wherein the presence or absence of one or more of the markers is indicative of the disease induced by the genome editing drug. In some embodiments, the sample of (a) and the sample of (b) are the same sample. In some embodiments, the sample of (a) and the sample of (b) are different samples. In some embodiments, an original sample was obtained from the subject, the sample of (a) is a first portion of the original sample, and the sample of (b) is a second portion of the original sample.

In some embodiments, the sequencing library and the sequencing panel are present in a single composition and/or are sequenced in a single sequencing cell. In some embodiments, the sequencing library and the sequencing panel are present in separate compositions and/or are sequenced separately.

In some embodiments, a method of screening for a disease induced by a genome editing drug and determining reduced efficacy of the genome editing drug is provided, comprising: (a) sequencing a sequencing panel comprising nucleic acid molecules obtained or derived from a sample from a subject that has undergone administration of the genome editing drug to generate sequencing reads; (b) determining a parameter comprising quantitative measures of levels of one or more proteins associated with the disease by performing a proteomic assay on molecules obtained or derived from a sample from the subject; (c) determining whether the first and the second parameters are within a predetermined therapeutic threshold, wherein the first and second parameters below the predetermined therapeutic threshold indicate an additional dose of the genome editing drug should be administered to the subject; and (d) detecting from the sequencing reads the presence or absence of one or more genetic, epigenetic, and/or fragmentomic markers, wherein the presence or absence of one or more of the markers is indicative of the disease induced by the genome editing drug. In some embodiments, the sample of (a) and the sample of (b) are the same sample. In some embodiments, the sample of (a) and the sample of (b) are different samples. In some embodiments, an original sample was obtained from the subject, the sample of (a) is a first portion of the original sample, and the sample of (b) is a second portion of the original sample.

In some embodiments, a method for determining therapeutic efficacy and adverse side effects of a genome editing drug is provided, comprising: (a) determining a first parameter comprising quantitative measures of levels of one or more proteins associated with the disease by performing a proteomic assay on molecules obtained or derived from a sample from a subject that has undergone administration of the genome editing drug; (b) determining a second parameter comprising quantitative measures of editing of one or more predetermined variants associated with the disease by sequencing a sequencing library comprising molecules obtained or derived from a sample from the subject; (c) determining whether the first and the second parameters are within a predetermined therapeutic threshold, wherein the first and second parameters below the predetermined therapeutic threshold indicate an additional dose of the genome editing drug should be administered to the subject; (d) sequencing a panel to generate sequencing reads from molecules obtained or derived from a third portion of the sample; and (e) detecting from the sequencing reads the presence or absence of one or more genetic, epigenetic, and/or fragmentomic markers, wherein the presence or absence of one or more of the markers is indicative of the adverse side effect induced by the genome editing drug. In some embodiments, the sample of (a) and the sample of (b) are the same sample. In some embodiments, the sample of (a) and the sample of (b) are different samples. In some embodiments, an original sample was obtained from the subject, the sample of (a) is a first portion of the original sample, and the sample of (b) is a second portion of the original sample.

In some embodiments, the genome editing drug comprises a CRISPR-Cas system that edits the one or more predetermined variants associated with the disease. In some embodiments, the genome editing drug further comprises one or more single guide RNAs (sgRNAs) that direct a Cas protein to the one or more predetermined variants associated with the disease. In some embodiments, the Cas protein is Cas9, dCas9, nCas9, or any combination thereof. In some embodiments, the CRISPR-Cas system introduces a gene knockout, gene insertion, a wildtype allele, an inactivating mutation, or any combination thereof.

In some embodiments, the sample is a bodily sample. In some embodiments, the sample is a blood sample or fractions thereof (e.g., plasma or serum), urine sample, or any combination thereof.

In some embodiments, the proteomic assay comprises mass spectrometry, an immunoassay, enzyme-linked immunosorbent assay (ELISA), High-performance liquid chromatography (HPLC), mass spectrometry, protein sequencing (e.g., ProtSeq), Immunohistochemistry (IHC), immunofluorescence, or any combination thereof.

In some embodiments, the genome editing drug is administered in vivo or ex vivo. In some embodiments, the ex vivo administration of the genome editing drug is to a stem cell, such as a hematopoietic stem cell. In some embodiments, the hematopoietic stem cells comprise a cell-based therapy. In some embodiments, the cell-based therapy comprises an immunotherapy.

In some embodiments, the disease is amyloidosis transthyretin-related (ATTR) or a hemoglobinopathy. In some embodiments, the one or more predetermine variants are within the TTR gene sequence. In some embodiments, the hemoglobinopathy is beta-thalassemia or sickle cell disease.

In some embodiments, the adverse side effect is cancer.

In some embodiments, at least a subset of the genetic, epigenetic, and/or fragmentomic markers are specific to the genome editing drug. In some embodiments, at least a subset of the genetic, epigenetic, and/or fragmentomic markers are specific to the subject based on the genomic profile of the subject. In some embodiments, the genetic, epigenetic, and/or fragmentomic markers further comprise one or more of a single nucleotide variant (SNV), insertion-deletion (indel), structural variant, methylation, histone acetylation, transcription factor binding site (TFBS), chromatin positioning, or nucleosome positioning.

In some embodiments, the nucleic acid molecules are DNA. In some embodiments, the DNA is cell-free DNA. In some embodiments, copies of the cell-free DNA molecules are generated prior to sequencing.

In some embodiments, one or more adapters comprising barcodes are attached to the nucleic acid molecules prior to sequencing. In some embodiments, the adapters are randomly attached to both ends of the nucleic acid molecules. In some embodiments, the adapters are randomly attached to either end of the nucleic acid molecules. In some embodiments, the nucleic acid molecules are uniquely barcoded. In some embodiments, the nucleic acid molecules are non-uniquely barcoded.

In some embodiments, a method of preparing a panel of hybridization probes is provided, comprising: (a) sequencing nucleic acid molecules obtained or derived from a sample from a subject that has undergone administration of a genome editing drug, thereby obtaining sequencing reads; (b) identifying a plurality of genomic locations with alterations using the sequencing reads; and (c) preparing the panel of hybridization probes, wherein the panel comprises hybridization probes specific for at least a portion of the plurality of genomic locations with alterations. In some embodiments, the hybridization probes are labeled with a binding partner. In some embodiments, the binding partner is biotin.

In some embodiments, a method of preparing a sequencing library is provided, comprising: (a) sequencing nucleic acid molecules obtained or derived from a sample from a subject that has undergone administration of a genome editing drug, thereby obtaining sequencing reads; (b) identifying a plurality of genomic locations with alterations using the sequencing reads; and (c) enriching a second sample from the subject for a plurality of target regions, wherein the target regions comprise at least a portion of the genomic locations identified in (b), thereby providing enriched target regions; (d) preparing a sequencing library comprising at least a portion of the enriched target regions.

In some embodiments, the enriching the second sample comprises targeted amplification of the plurality of target regions. In some embodiments, the enriching the second sample comprises capturing the plurality of target regions with a plurality of hybridization probes.

In some embodiments, the preparing the sequencing library comprises adding adapters to at least a portion of the enriched target regions. In some embodiments, the adapters comprise barcodes. In some embodiments, the preparing the sequencing library comprises amplifying the enriched target regions.

In some embodiments, the alterations comprise on-target genetic alterations, off-target genetic alterations, epigenetic alterations, fragmentomic alterations, or any combination thereof. In some embodiments, the alterations are relative to a wild-type reference genome. In some embodiments, the alterations are relative to the genome of the subject before administration of the genome editing drug.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### C. Subjects

In some embodiments, the DNA (e.g., cfDNA or DNA from a sample comprising cells) is obtained from a subject (e.g., a test subject) having a cancer or a precancer. In some embodiments, the subject has a stage I cancer, stage II cancer, stage III cancer, or stage IV cancer. In some embodiments, the DNA from the subject is obtained and/or derived from a sample obtained from the subject. In some embodiments, the DNA is obtained from a subject suspected of having a cancer or a precancer. In some embodiments, the DNA is obtained from a subject having a tumor. In some embodiments, the DNA is obtained from a subject suspected of having a tumor. In some embodiments, the DNA is obtained from a subject having neoplasia. In some embodiments, the DNA is obtained from a subject suspected of having neoplasia. In some embodiments, the DNA is obtained from a subject in remission from a tumor, cancer, or neoplasia (e.g., following chemotherapy, surgical resection, radiation, or a combination thereof). In any of the foregoing embodiments, the precancer, cancer, tumor, or neoplasia or suspected precancer, cancer, tumor, or neoplasia may be of the bladder, head or neck, lung, colon, rectum, kidney, breast, prostate, skin, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject. In any of the foregoing embodiments, the subject may be a test subject.

### D. Samples

A sample can be any biological sample isolated from a subject. Samples can include body tissues, such as known or suspected solid tumors (such as carcinomas, adenocarcinomas, or sarcomas), whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof (e.g., plasma and/or serum), and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids or other nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, post-translation modifications (PTMs) of chromatin, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma can depend on the desired read depth for sequenced regions. Exemplary volumes are 0.4-40 mL, 5-20 mL, 10-20 mL. For examples, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be for example 5 to 20 mL. In some embodiments, the sample volume is 3-5 mL of plasma, such as 4 mL of plasma, per 10 mL whole blood.

A sample can comprise various amount of nucleic acid that contains genome equivalents. For example, a sample of about 30 ng DNA can contain about 10,000 haploid human genome equivalents and, in the case of cell-free DNA, about 200 billion individual nucleic acid molecules. Similarly, a sample of about 100 ng of DNA can contain about 30,000 haploid human genome equivalents and, in the case of cell-free DNA, about 600 billion individual molecules. Some samples contain 1-500, 2-100, 5-150 ng cell-free DNA, e.g., 5-30 ng, or 10-150 ng cell-free DNA.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

A sample can comprise various amounts of nucleic acid that contain genome equivalents. For example, a sample of about 30 ng DNA can contain about 10,000 (104) haploid human genome equivalents. Similarly, a sample of about 100 ng of DNA can contain about 30,000 haploid human genome equivalents.

A sample can comprise nucleic acids from different sources. For example, a sample can comprise germline DNA or somatic DNA. A sample can comprise nucleic acids carrying mutations. For example, a sample can comprise DNA carrying germline mutations and/or somatic mutations. Somatic mutations refer to mutations originating in somatic cells of a subject, e.g., precancer cells or cancer cells. A sample can also comprise DNA carrying cancer-associated mutations (e.g., cancer-associated somatic mutations), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids or other nucleic acids (e.g., cell-free DNA from a buffy coat sample or any other sample comprising cells, such as a blood sample (e.g., a whole blood sample, a leukapheresis sample, or a PBMC sample)) in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acid molecules. The method can comprise obtaining 1 femtogram (fg) to 200 ng.

An exemplary sample is 5-10 ml of whole blood, plasma or serum, which includes about 30 ng of DNA or about 10,000 haploid genome equivalents.

Cell-free nucleic acids are nucleic acids not contained within or otherwise bound to a cell or in other words nucleic acids remaining in a sample of removing intact cells. Cell-free nucleic acids or other nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids or other nucleic acids can be double-stranded, single-stranded, or a hybrid thereof. Double-stranded DNA molecules at least some of which have single-stranded overhangs are a preferred form of cell-free DNA for any method disclosed herein. A cell-free nucleic acid or other nucleic acid can be released into bodily fluid through secretion or cell death processes, e.g., cellular necrosis and apoptosis. Some cell-free nucleic acids or other nucleic acids are released into bodily fluid from cancer cells e.g., circulating tumor DNA, (ctDNA). Others are released from healthy cells.

A cell-free nucleic acid or other nucleic acid can have one or more epigenetic modifications, for example, a cell-free nucleic acid or other nucleic acid can be acetylated, methylated, ubiquitinylated, phosphorylated, sumoylated, ribosylated, and/or citrullinated.

Cell-free nucleic acids or other nucleic acids can have a size distribution of about 100-500 nucleotides, particularly 110 to about 230 nucleotides, with a mode of about 168 nucleotides and a second minor peak in a range between 240 to 440 nucleotides.

Cell-free nucleic acids or other nucleic acids can be isolated from bodily fluids through a partitioning step in which cell-free nucleic acids or other nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield.

After such processing, samples can include various forms of nucleic acid including double-stranded DNA, single-stranded DNA and single-stranded RNA. Optionally, single stranded DNA and RNA can be converted to double stranded forms so they are included in subsequent processing and analysis steps.

Reference or control molecules can be added to or spiked into a sample as a control or normalization standard. For example, a certain amount of modified DNA from a species other than the species of the subject from which the sample was obtained or synthetic nucleic acids comprising certain modifications may be added to the sample. In some embodiments, the reference or control molecules are distinguishable from the molecules originally present in the sample. In some embodiments, the detected DNA sequences are normalized to the reference or control molecules.

In some embodiments, a plurality of first subsamples (e.g., from different subjects, and/or which have been distinguishably tagged with sample tags) are pooled before a sequencing step. This approach can reduce costs, e.g., in that less reagent may be needed per subsample treated.

DNA molecules can be linked to adapters at either one end or both ends. Typically, double-stranded molecules are blunt ended by treatment with a polymerase with a 5′-3′ polymerase and a 3′-5' exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

E. Subjecting the DNA or a Subsample Thereof to a Procedure that Affects a First Nucleobase in the DNA Differently from a Second Nucleobase in the DNA

In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. Such a step can be useful, e.g., for facilitating identification of epigenetic features of the DNA being sequenced, including epigenetic alterations that may be markers of an adverse side effect of the genome editing drug. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA before library preparation using the DNA, before a first amplification of the DNA, before dividing the DNA into a plurality of subsamples, or any combination thereof. In certain embodiments, the DNA is subjected to the procedure before or after contacting the DNA with a methylation-sensitive nuclease.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA of the first subsample comprises bisulfite conversion. Treatment with bisulfite converts unmodified cytosine and certain modified cytosine nucleotides (e.g., 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, thus facilitates identifying positions containing mC or hmC using the sequence reads obtained from the exemplary sample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA of the first subsample comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336:934-937.

In some embodiments, the procedure that affects a first nucleobase in the DNA differently from a second nucleobase in the DNA of the first subsample comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149:1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA of the first subsample comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

Alternatively, protection of hmC (e.g., using BGT or 5-hydroxymethylcytosine carbamoyltransferase) can be combined with Tet-assisted conversion with a substituted borane reducing agent. hmC can be protected as noted above through glucosylation using BGT, forming ghmC, or through carbamoylation using 5-hydroxymethylcytosine carbamoyltransferase, forming 5cmC. Treatment with a TET protein such as mTet1 then converts mC to caC but does not convert C, ghmC, or 5cmC. caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA of the first subsample comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO₄) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12 (17): e4496.

In some embodiments, the procedure that affects a first nucleobase in the DNA differently from a second nucleobase in the DNA of the first subsample comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12 (17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA of the first subsample comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form β-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63:711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N⁶-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

In some embodiments, the conversion procedure is an enzymatic conversion procedure which converts the base pairing specificity of modified nucleosides (e.g., DM-seq conversion comprising adding a protective group (such as a carboxymethyl group) to unmodified cytosines, and deaminating 5mC, such as using an APOBEC enzyme) or an enzymatic conversion procedure which converts the base pairing specificity of unmodified nucleosides (such as SEM-seq).

In some cases, the conversion procedure used in the methods of the disclosure is one that changes the base pairing specificity of a modified nucleoside (e.g., methylated cytosine), but does not change the base pairing specificity of the corresponding unmodified nucleoside (e.g., cytosine) or does not change the base pairing specificity of any un-modified nucleoside (e.g., cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as DM-seq may in some cases be preferred over methods such as bisulfite sequencing and EM-seq because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized region of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on % of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g., cytosine), but does not change the base pairing specificity of the corresponding modified nucleoside (e.g., methylated cytosine).

The skilled person can select a suitable method according to their needs, including which nucleoside modifications are to be detected and/or identified.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI having SEQ ID NO: 1 or SEQ ID NO: 2, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase of SEQ ID NO: 1 or SEQ ID NO: 2 can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using BGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (BGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149:1368-80, and in Yang et al., Bio-protocol, 2023; 12 (17): c4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein (SEQ ID NO: 3 in the present application). In some embodiments, the one or more TET enzymes comprise TETcd. TETcd is described in U.S. Pat. No. 10,260,088 and its sequence is SEQ ID NO: 3 therein (SEQ ID NO: 4 in the present application). In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker (SEQ ID NO: 5 of the present application) as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V1900I, or V1900P TET2 mutant. Examples of V1900A, V1900C, V1900G, V1900I, and V1900P TET2 mutants are provided as SEQ ID NOs: 6-10. In some embodiments, the V1900 TET mutant has at least 80%, 85%, 90%, 95%, 96%, 97%, 98%, or 99% sequence identity to SEQ ID NO: 6, 7, 8, 9, or 10. Position 1900 of the wild-type TET2 sequence corresponds to position 438 in each of SEQ ID NOs: 5-10. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S are provided as SEQ ID NOs: 11 and 12. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13 (2): 181-187. As demonstrated in Liu et al., TET2 comprising a T1372S mutation can more efficiently oxidize 5mC to produce 5-carboxylcytosine (5caC) than other versions of TET2 such as TET2 lacking a T1372S mutation. In some embodiments, the TET2 enzyme comprises SEQ ID NO: 14 or optionally a variant of SEQ ID NO: 14 in which at least 5, 6, 7, or 8 positions match SEQ ID NO: 14 including position 5 of SEQ ID NO: 14. In some embodiments, the TET2 enzyme is a human TET2 enzyme comprising a T1372S mutation. In some embodiments, the TET2 enzyme comprises the sequence of SEQ ID NO: 11. In some embodiments, the TET2 enzyme comprises a sequence having at least 80%, 85%, 90%, 95%, 96%, 97%, 98%, or 99% identity to SEQ ID NO: 11. In some embodiments, the TET2 enzyme comprises a sequence having at least 80%, 85%, 90%, 95%, 96%, 97%, 98%, or 99% identity to SEQ ID NO: 12. In some embodiments, the TET2 enzyme comprises the sequence of SEQ ID NO: 12. The sequences of SEQ ID NOs: 11 and 12 are shown below.

Provided herein is a method comprising contacting DNA contacting DNA with a TET2 enzyme comprising a T1372S mutation to oxidize 5-methylcytosine (5mC) and/or 5-hydroxymethylcytosine (5hmC) present in the DNA to 5-carboxycytosine (5caC), subsequently contacting at least a portion of the DNA with a substituted borane reducing agent, thereby converting 5-caC in the DNA to dihydrouracil (DHU), thereby producing treated DNA, and sequencing at least a portion of the treated DNA.

The following Table 1 shows exemplary sequences useful in certain disclosed methods.

TABLE 1

| SEQ ID NQ | |
|---|---|
| and | |
| description | Sequence |
| SEQ ID NO: 1 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGKSIPVKILEAIFNTLEFVNNEELE |
| SEQ ID NO: 2 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGRSIPVKILEAIFNTLEFVNNEELE |
| SEQ ID NO: 3 | GGSQSQNGKCEGCNPDKDEAPYYTHLGAGPDVAAIRTLMEERYGEKGKAIRIEKVIYTGKE |
| TETV | GKSSQGCPIAKWVYRRSSEEEKLLCLVRVRPNHTCETAVMVIAIMLWDGIPKLLASELYSE |
| | LTDILGKCGICTNRRCSQNETRNCCCQGENPETCGASFSFGCSWSMYYNGCKFARSKKPRK |
| | FRLHGAEPKEEERLGSHLQNLATVIAPIYKKLAPDAYNNQVEFEHQAPDCCLGLKEGRPFS |
| | GVTACLDFSAHSHRDQQNMPNGSTVVVTLNREDNREVGAKPEDEQFHVLPMYIIAPEDEFG |
| | STEGQEKKIRMGSIEVLQSFRRRRVIRIGELPKSCEVSGQDAAAVQEIEYWSDSEHNFQDP |
| | CIGGVAIAPTHGSILIECAKCEVHATTKVNDPDRNHPTRISLVLYRHKNLFLPKHCLALWE |
| | AKMAEKARKEEECGKNGSDHVSQKNHGKQEKREPTGPQEPSYLRFIQSLAENTGSVTTDST |
| | VTTSPYAFTQVTGPYNTFV |
| SEQ ID NO: 4 | QSQNGKCEGCNPDKDEAPYYTHLGAGPDVAAIRTLMEERYGEKGKAIRIEKVIYTGKEGKS |
| TETcd | SQGCPIAKWVYRRSSEEEKLLCLVRVRPNHTCETAVMVIAIMLWDGIPKLLASELYSELTD |
| | ILGKCGICTNRRCSQNETRNCCCQGENPETCGASFSFGCSWSMYYNGCKFARSKKPRKFRL |
| | HGAEPKEEERLGSHLQNLATVIAPIYKKLAPDAYNNQVEFEHQAPDCCLGLKEGRPFSGVT |
| | ACLDFSAHSHRDQQNMPNGSTVVVTLNREDNREVGAKPEDEQFHVLPMYIIAPEDEFGSTE |
| | GQEKKIRMGSIEVLQSFRRRRVIRIGELPKSCKKKAEPKKAKTKKAARKRSSLENCSSRTE |
| | KGKSSSHTKLMENASHMKQMTAQPQLSGPVIRQPPTLQRHLQQGQRPQQPQPPQPQPQTTP |
| | QPQPQPQHIMPGNSQSVGSHCSGSTSVYTRQPTPHSPYPSSAHTSDIYGDTNHVNFYPTSS |
| | HASGSYLNPSNYMNPYLGLLNQNNQYAPFPYNGSVPVDNGSPFLGSYSPQAQSRDLHRYPN |
| | QDHLTNQNLPPIHTLHQQTFGDSPSKYLSYGNQNMQRDAFTTNSTLKPNVHHLATFSPYPT |
| | PKMDSHFMGAASRSPYSHPHTDYKTSEHHLPSHTIYSYTAAASGSSSSHAFHNKENDNIAN |
| | GLSRVLPGFNHDRTASAQELLYSLTGSSQEKQPEVSGQDAAAVQEIEYWSDSEHNFQDPCI |
| | GGVAIAPTHGSILIECAKCEVHATTKVNDPDRNHPTRISLVLYRHKNLFLPKHCLALWEAK |
| | MAEKARKEEECGKNGSDHVSQKNHGKQEKREPTGPQEPSYLRFIQSLAENTGSVTTDSTVT |
| | TSPYAFTQVTGPYNTFV |
| SEQ ID NO: 5 | MDYKDDDDKHMGGSDFPSCRCVEQIIEKDEGPFYTHLGAGPNVAAIREIMEERFGQKGKA |
| wild type | IRIERVIYTGKEGKSSQGCPIAKWVVRRSSSEEKLLCLVRERAGHTCEAAVIVILILVWE |
| TET2 | GIPLSLADKLYSELTETLRKYGTLTNRRCALNEERTCACQGLDPETCGASFSFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLVFYQHKSMNEPKHGLALWEAKMAEKAREKEEECEKYG |
| SEQ ID NO: 6 | MDYKDDDDKHMGGSDFPSCRCVEQIIEKDEGPFYTHLGAGPNVAAIREIMEERFGQKGKA |
| TET2 V1900A | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLAFYQHKSMNEPKHGLALWEAKMAEKAREKEEECEKYG |
| SEQ ID NO: 7 | MDYKDDDDKHMGGSDFPSCRCVEQIIEKDEGPFYTHLGAGPNVAAIREIMEERFGQKGKA |
| TET2 V1900C | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLCFYQHKSMNEPKHGLALWEAKMAEKAREKEEECEKYG |
| SEQ ID NO: 8 | MDYKDDDDKHMGGSDFPSCRCVEQIIEKDEGPFYTHLGAGPNVAAIREIMEERFGQKGKA |
| TET2 V1900G | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLGFYQHKSMNEPKHGLALWEAKMAEKAREKEEECEKYG |
| SEQ ID NO: 9 | MDYKDDDDKHMGGSDFPSCRCVEQIIEKDEGPFYTHLGAGPNVAAIREIMEERFGQKGKA |
| TET2 V1900I | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLIFYQHKSMNEPKHGLALWEAKMAEKAREKEEECEKYG |
| SEQ ID NO: 10 | MDYKDDDDKHMGGSDFPSCRCVEQIIEKDEGPFYTHLGAGPNVAAIREIMEERFGQKGKA |
| TET2 V1900P | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASESFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLPFYQHKSMNEPKHGLALWEAKMAEKAREKEEECEKYG |
| SEQ ID NO: 11 | MDYKDDDDKHMGGSDFPSCRCVEQIIEKDEGPFYTHLGAGPNVAAIREIMEERFGQKGKAI |
| TET2-CS- | RIERVIYTGKEGKSSQGCPIAKWVVRRSSSEEKLLCLVRERAGHTCEAAVIVILILVWEGI |
| T1372S | PLSLADKLYSELTETLRKYGTLTNRRCALNEERTCACQGLDPETCGASFSFGCSWSMYYNG |
| | CKFARSKIPRKFKLLGDDPKEEEKLESHLQNLSTLMAPTYKKLAPDAYNNQIEYEHRAPEC |
| | RLGLKEGRPFSGVSACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLVFYQHKSMNEPKHGLALWEAKMAEKAREKEEECEKYG |
| SEQ ID NO: 12 | MGGSDFPSCRCVEQIIEKDEGPFYTHLGAGPNVAAIREIMEERFGQKGKAIRIERVIYTGK |
| TET2-CD- | EGKSSQGCPIAKWVVRRSSSEEKLLCLVRERAGHTCEAAVIVILILVWEGIPLSLADKLYS |
| T1372S | ELTETLRKYGTLTNRRCALNEERTCACQGLDPETCGASFSFGCSWSMYYNGCKFARSKIPR |
| | KFKLLGDDPKEEEKLESHLQNLSTLMAPTYKKLAPDAYNNQIEYEHRAPECRLGLKEGRPF |
| | SGVSACLDFCAHAHRDLHNMQNGSTLVCTLTREDNREFGGKPEDEQLHVLPLYKVSDVDEF |
| | GSVEAQEEKKRSGAIQVLSSFRRKVRMLAEPVKTCRQRKLEAKKAAAEKLSSLENSSNKNE |
| | KEKSAPSRTKQTENASQAKQLAELLRLSGPVMQQSQQPQPLQKQPPQPQQQQRPQQQQPHH |
| | PQTESVNSYSASGSTNPYMRRPNPVSPYPNSSHTSDIYGSTSPMNFYSTSSQAAGSYLNSS |
| | NPMNPYPGLLNQNTQYPSYQCNGNLSVDNCSPYLGSYSPQSQPMDLYRYPSQDPLSKLSLP |
| | PIHTLYQPRFGNSQSFTSKYLGYGNQNMQGDGFSSCTIRPNVHHVGKLPPYPTHEMDGHFM |
| | GATSRLPPNLSNPNMDYKNGEHHSPSHIIHNYSAAPGMENSSLHALHLQNKENDMLSHTAN |
| | GLSKMLPALNHDRTACVQGGLHKLSDANGQEKQPLALVQGVASGAEDNDEVWSDSEQSFLD |
| | PDIGGVAVAPTHGSILIECAKRELHATTPLKNPNRNHPTRISLVFYQHKSMNEPKHGLALW |
| | EAKMAEKAREKEEECEKYGPDYVPQKSHGKKVKREPAEPHETSEPTYLRFIKSLAERTMSV |
| | TTDSTVTTSPYAFTRVTGPYNRYI |
| SEQ ID NO: 13 | FSGVTACLD |
| SEQ ID NO: 14 | FSGVSACLD |
| TET2 T1372S | |

### F. Linking Sample Nucleic Acid Molecules to Adapters

Nucleic acids present in a sample with or without prior processing as described above typically contain a substantial portion of molecules in the form of partially double-stranded molecules with single-stranded overhangs. Such molecules can be converted to blunt-ended double-stranded molecules by treating with one or more enzymes to provide a 5′-3′ polymerase and a 3′-5′ exonuclease (or proof-reading function), in the presence of all four standard nucleotide types. Such a combination of activities can extend strands with a recessed 3′ end so they end flush with 5′ end of the opposing strand (in other words generating a blunt end) or can digest strands with 3′ overhangs so they are likewise flush with 5′ end of the opposing strand. Both activities can optionally be conferred by a single polymerase. The polymerase is preferably heat-sensitive so that its activity can be terminated when the temperature is raised. Klenow large fragment and T4 polymerase are examples of suitable polymerase.

The one or more enzymes conferring 5′-3′ polymerase and a 3′-5′ exonuclease activity are preferably denatured by raising the temperature or otherwise. For example, denaturation can be affected by raising the temperature to e.g., to 75°−80° C. The samples are then acted on by a polymerase lacking a proof-reading function. This polymerase is preferably thermostable such as to remain active at the elevated temperature. Taq, Bst large fragment and Tth polymerases are examples of such a polymerase. The second polymerase effects a non-templated addition of a single nucleotide to the 3′ ends of blunt-ended nucleic acids. Although the reaction mixture typically contains equal molar amounts of each of the four standard nucleotide types from the prior step, the four nucleotide types are not added to the 3′ ends in equal proportions. Rather A is added most frequently, followed by G followed by C and T.

In certain embodiments, after tailing of the sample molecules, and with or without subsequent purification of the tailed sample molecules, the tailed sample molecules are contacted with adapters tailed with complementary T and C nucleotides at one end of the adapters. Adapters are typically formed by separate synthesis and annealing of their respective strands. The additional T and C tails can thus be added as an extra nucleotide in synthesis of one of the strands. Typically adapters tailed with G and A are not included because although these adapters might anneal with sample molecules tailed with C and T respectively, they would also anneal with other adapters. Adapter molecules and sample molecules bearing complementary nucleotides (i.e., T-A and C-G) at their 3′ ends anneal and can be ligated to one another. The percentage of C-trailed adapters relative to T-tailed adapters ranges from about 5-40% by moles, for example, 10-35%, 15-25%, 20-35%, 25-35% or about 30%. Because the non-template directed addition of a single nucleotide to the 3′ ends of sample molecules does not proceed to completion, the sample also contains some blunt-ended sample molecules without tailing. These molecules can be recovered by also supplying the sample with adapters having one and preferably only one blunt end. Blunt end adapters are usually supplied at a molar ratio of 0.2-20%, or 0.5-15% or 1-10% of adapters with T- and C-tailed adapters. Blunt-ended adapters can be provided at the same time, before or after the T- and C-tailed adapters. Blunt-ended adapters ligated with blunt-ended sample molecules again resulting in sample molecules flanked on both sides by adapters. These molecules lack the A-T or C-G nucleotide pairs between sample and adapters present when tailed sample molecules are ligated to tailed adapters.

The adapters used in these reactions preferably have one and only one end tailed with T or C or one and only one end blunt so that they can ligate with sample molecules in only one orientation. The adapters can be for example Y-shaped adapters in which one end is tailed or blunt and the other end has two single strands. Exemplary Y-shaped adapters have sequences as follows with (6 bases) indicating a tag. The upper oligonucleotide includes a single base T tail.

Customized combinations of such oligonucleotide including oligonucleotides with both T and C tails can be synthesized for use in the present methods.

A truncated version of these adapter sequences has been described by Rohland et al., Genome Res. 2012 May; 22 (5): 939-946.

Adapters can also be bell-shaped with only one end, which is tailed or blunt. Adapters can include a primer binding site for amplification, a binding site for a sequencing primer, and/or a nucleic acid tag for purposes of identification. The same or different adapters can be used in in a single reaction.

When adapters include an identification tag and nucleic acids in a sample are attached to adapters at each end, the number of potential combinations of identifiers increases exponentially with the number of unique tags supplied (i.e., nⁿ combinations, where n is the number of unique identification tags). In some methods, the number of combinations of unique tags is sufficient that it is statistically probable that all or substantially all (e.g., at least 90%) of different double-stranded DNA molecules in the sample receive a different combination of tags. In some methods, the number of unique combinations of identifier tags is less than the number of unique double-stranded DNA molecules in the sample (e.g., 5-10,000 different tag combinations).

A kit providing suitable enzymes for performing the above methods is the NEBNext® Ultra™ II DNA Library Prep Kit for Illumina®. The kit provides the following reagents

NEBNext Ultra II End Prep Enzyme Mix, NEBNext Ultra II End Prep Reaction Buffer, NEBNext Ligation Enhancer, NEBNext Ultra II Ligation Master Mix-20, NEBNext® Ultra II Q5® Master Mix.

The blunt-ending and tailing of sample nucleic acids can be performed in a single-tube. Blunt-ended nucleic acids need not be separated from the enzyme(s) performing the blunt ending before the tailing reaction occurs. Optionally, all enzymes, nucleotides and other reagents are supplied together before the blunt ending reaction occurs. Supplying together means that all are introduced in the sample sufficiently proximate in time such that all are present when the sample incubation occurs for blunt ending to take place. Optionally, nothing is removed from the samples after supplying the enzymes, nucleotides and other reagents at least until both the blunt ending and end tailing incubations have been completed. Often, the end tailing reaction is performed at a higher temperature than the blunt ending reaction. For example, the blunt ending reaction can be performed at ambient temperature in which 5′-3′ polymerase and 3′-5′ exonuclease are active and the thermostabile polymerase is inactive or minimally active, and the end tailing reaction performed at an elevated temperature, such as over 60° C., when 5′-3′ polymerase and 3′-5′ exonuclease are inactive and the thermostabile polymerase is active.

DNA, e.g., after end-repair, can be subjected to blunt-end ligation with blunt-ended adapters, in cases where A-tailing is not performed, or sticky end ligation with T-tailed adapters, when A tailing is performed. DNA molecules can be ligated to adapters at either one end or both ends. DNA molecules can be ligated with at least partially double stranded adapter (e.g., a Y shaped or bell-shaped adapter). In embodiments wherein the modification-sensitive sequencing comprises a conversion procedure, the ligation step can take place before or after the conversion step. In some embodiments, the ligation step is performed after the conversion step. In some embodiments, adapters are ligated to end-repaired DNA molecules or the adapters are ligated to the plurality of DNA molecules. In some such embodiments, the ligation reaction also seals nicks present in the end-repaired DNA.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample DNA molecule. In some instances, two adapters can be ligated to a single sample DNA molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample DNA molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given DNA molecule. Adapters of the same or different sequence can be linked to the respective ends of a sample DNA molecule. In some cases, adapters of the same or different sequence are linked to the respective ends of the DNA molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a DNA molecule to be analyzed. Other exemplary adapters include T-tailed, C-tailed or hairpin shaped adapters. For example, a hairpin shaped adaptor can comprise a complementary double stranded portion and a loop portion, where the double stranded portion can be attached (e.g., ligated) to a double-stranded polynucleotide. Hairpin shaped sequencing adaptors can be attached to both ends of a polynucleotide fragment to generate a circular molecule, which can be sequenced multiple times.

In some embodiments, the nucleic acids further comprise adapters in which at least one cytosine is a modification resistant cytosine, optionally wherein each cytosine in the adapters is a modification resistant cytosine. In some embodiments, methods further comprise further comprising ligating adapters to the nucleic acids, wherein at least one cytosine in the adapters is a modification resistant cytosine, optionally wherein each cytosine in the adapters is a modification resistant cytosine. The adapters may comprise barcodes, e.g., according to any of the embodiments relating to barcodes described elsewhere herein. In some embodiments, the adapters can include at least 1, 2, 3, 4, 5, 6, 7, 8, 9, 10, 11, 12, 13, 14, 15, 16, 17, 18, 19, or 20 modified nucleotides, such as modified cytosine nucleotides, that are resistant to modification, e.g., conversion. In some embodiments, the modified nucleotides are resistant to modification by a deaminase. In some embodiments, the modified nucleotides comprise a conversion resistant modified cytosine, such as 5-propynylC (5pyC), 5-pyrrolo-dC (5pyrC), 5-hydroxymethylcytosine (5hmC) along with modified variants thereof, glucosylated5-hydroxymethylcytosine (5ghmC), cytosine 5-methylenesulfonate (CMS), bulky 5-position adducts, or N4-modified cytosine. In some embodiments, the conversion resistant modified cytosine is 5pyC, 5pyrC, 5ghmC, or CMS. In some embodiments, the conversion resistant modified cytosine can protect cytosine from being converted by a deaminase, such as a cytidine deaminase, which converts a cytosine to uracil. In some embodiments, each cytosine of an adapter is a conversion resistant modified cytosine, such as any one or more of the foregoing examples. For exemplary descriptions of modified nucleotides and their use in adaptors, see WO2023/288222 and U.S. Pat. No. 10,260,088.

The adapters used in the methods of the present disclosure comprise one or more known modified nucleosides, such as methylated nucleosides. In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure.

In some embodiments, adapters may be added to the DNA or a subsample thereof. Adapters can be ligated to DNA at any point in the methods herein. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

### G. Amplification

Sample nucleic acids flanked by adapters can be amplified by PCR and other amplification methods typically primed from primers binding to primer binding sites in adapters flanking a nucleic acid to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence based amplification, and self-sustained sequence based replication.

Preferably, the present methods result in at least 75, 80, 85, 90 or 95% of double-stranded nucleic acids in the sample being linked to adapters. Preferably use of T- and C-tailing increases the percentage of double-stranded nucleic acids in the sample linked to adaptors relative to control methods performed with T-tailed adapters alone by at least 1, 2, 3, 4, 5, 6, 7, 8, 9 or 10% (an increase of yield from 75% to 80% being considered a 5% increase). Preferably, use of T- and C-tailing in combination with blunt-ended adaptors increase the percentage of double-stranded nucleic acids linked to adaptors by at least 5, 10, 15, 20 or 25%. The percentage of nucleic acids linked to adaptors can be determined by comparative gel electrophoresis of the original sample and the processed sample after linkage to adapters has been completed.

Preferably, the present methods result in at least 75, 80, 85, 90 or 95% of available double-stranded molecules in the sample being sequenced. Preferably the use of T- and C-tailing increases the percentage of double-stranded nucleic acids in the sample being sequenced relative to control methods performed with T-tailed adapters alone by at least 1, 2, 3, 4, 5, 6, 7, 8, 9 or 10%. Preferably the use of T- and C-tailing in combination with blunt ended adaptors increases the percentage of double-stranded nucleic acid in the sample being sequenced relative to control methods performed with T-tailed adaptors along by at least 5, 10, 15, 20 or 25%. The percentage of nucleic acids being sequenced can be determined by comparing the number of molecules actually sequenced based on the number that could have been sequenced based on the input nucleic acids and regions of the genome targeted for sequencing.

Adapted DNA can be amplified (e.g., by PCR) prior to, or as part of, the modification-sensitive sequencing. For example, in modification-sensitive sequencing procedures which comprise a conversion step, the adapted DNA may be amplified after the conversion step. In modification-sensitive sequencing procedures which involve single molecule sequencing (such a nanopore-based sequencing or SMRT sequencing), there may be no amplification step. In some embodiments, the DNA amplification step is performed after a step of subjecting a DNA sample to end repair and before a step of subjecting the end-repaired DNA molecules to modification-sensitive sequencing.

Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. For example, DNA flanked by adapters added to the DNA as described herein can be amplified by PCR or other amplification methods. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids. The present methods can increase the amount or number of amplified molecules relative to control methods performed with T-tailed adapters alone by at least 10, 15, or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

### H. Enriching, Capturing, and Using Capture Probes

DNA molecules in a sample can be subject to a capture step, in which molecules having target sequences are captured for subsequent analysis. In some embodiments, methods disclosed herein comprise a step of capturing one or more sets of target regions of DNA, such as cfDNA. In some embodiments, the capture step (also referred to herein as an “enriching” or “enrichment” step) is performed prior to sequencing; where DNA is subjected to a procedure to a procedure that affects a first nucleobase in the DNA differently from a second nucleobase in the DNA, the capture step may be performed before or after such a step (noting that when performed afterward, capture probe sequences can account for base conversions that may have occurred). A capture step can use a panel of hybridization probes prepared as described herein, e.g., as oligonucleotide baits. The oligonucleotide baits may further comprise additional oligonucleotides targeting one or more sets of target regions as described elsewhere herein. Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

A panel of regions targeted for enrichment can be selected such that they do not contain regions known to include the base modification used in the end repair reaction. When the end repair is performed with dNTPs comprising 5mC or 5hmC, a panel of regions targeted for enrichment may be selected such that they do not contain CpH dinucleotides which are known to be naturally methylated in the subject (e.g., humans). Such CpH dinucleotides can be identified through the use of publicly available resources (e.g., MethBank3.0: a database of DNA methylomes across a variety of species Nucleic Acids Res 2018). Such an approach has the advantage that any detected methylated CpH dinucleotides can unambiguously be attributed to regions synthesized in the end repair.

In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein.

In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater than the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

### 1. Enriched Set

In some embodiments, a captured set of DNA (e.g., cfDNA) is provided. With respect to the disclosed methods, the captured set of DNA may be provided, e.g., by performing a capturing step prior to a sequencing step as described herein. The captured set may comprise DNA corresponding to a sequence-variable target region set, an epigenetic target region set, or a combination thereof. In some embodiments, a capture step is performed prior to a conversion step or after a conversion step.

In some embodiments, a first target region set is captured (e.g., from a sample or a first subsample), comprising at least epigenetic target regions. The epigenetic target regions captured from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that show lower methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is captured from the second subsample, comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The first and second captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-1 kb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion. and fusions comprising any of ALK, FGFR2, FGFR3, NTRK1, RET, or ROS1.

In some embodiments, the DNA that is captured comprises target regions having a type-specific epigenetic variation. In some embodiments, an epigenetic target region set consists of target regions having a type-specific epigenetic variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, the DNA that is captured comprises target regions having a type-specific epigenetic variation. In some embodiments, an epigenetic target region set consists of target regions having a type-specific epigenetic variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in pre-cancerous or cancerous colon tissue. Examples of such target regions are shown in Table 2 below.

TABLE 2
Hypermethylated target regions with aberrantly
high copy number in colon cancer or pre-cancer

| Chromosomal | |
|---|---|
| region with | Genes comprising DMRs within |
| copy number gain | the chromosomal region |
| 7p21.3-15.1 | VWDE, TWIST1, DNAH1, GPNMB, NPY, |
| | GSDME, NFE2L3, HOXA1, HOXA7, EVX1, |
| | CREB5 |
| 8q13.3-24.3 | PRDM14, MSC, TRPA1, SBSPON, CRISPLD1, |
| | ZFHX4, RALYL, MMP16, C8orf88, RUNX1T1, |
| | CDH17, GDF6, SDC2, OSR2, PABPC1, ZNF706, |
| | RIMS2, ZFPM2, SYBU, CSMD3, TRPS1, AARD, |
| | TNFRSF11B, MAL2, COL14A1, POU5F1B, ADCY8, |
| | ST3GAL1, TRAPPC9, AGO2, ADGRB1, LY6E, |
| | LY6H, GLI4, MAFA, TSTA3, PLEC, OPLAH, |
| | FOXH1 |
| 13q14.1-33.3 | ELF1, RGCC, TNFSF11, SERP2, RB1, CNMD, |
| | PCDH17, EDNRB, POU4F1, SLITRK5, GPC6, |
| | SOX21, CLDN10, STK24, ZIC2, NALCN, FGF14, |
| | MYO16, COL4A1, COL4A2, SOX1, F7 |
| 20q12-13.33 | MAFB, LPIN3, EMILIN3, PTPRT, TOX2, GDAPIL1, |
| | HNF4A, MMP9, CDH22, PREX1, ZNFX1, PARD6B, |
| | ZFP64, DOK5, FAM210B, TFAP2C, RBM38, |
| | CTCFL, GNAS, PHACTR3, CDH4, GATA5, |
| | BHLHE23, YTHDF1, NKAIN4, CHRNA4, KCNQ2, |
| | GMEB2 |

TABLE 3
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type.

Without wishing to be bound by any particular theory, in an individual with cancer, proliferating or activated immune cells and/or dying cancer cells may shed more DNA into the bloodstream than immune cells in a healthy individual and/or healthy cells of the same tissue type, respectively. As such, the distribution of cell type and/or tissue of origin of cfDNA may change upon carcinogenesis. Thus, the presence and/or levels of cfDNA originating from certain cell or tissue types can be an indicator of disease. Variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.cu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. The sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLOS One 13: c0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

I. Contacting DNA with a Methylation-Sensitive or Methylation-Dependent Nuclease

In some embodiments, DNA or a subsample thereof (e.g., a first, second, or third subsample prepared by partitioning a sample as described herein, such as on the basis of a level of a cytosine modification, such as methylation, e.g., 5-methylation) is contacted with a methylation-dependent nuclease or methylation-sensitive nuclease. The contacting can be performed using a sample that has been divided into a plurality of subsamples as disclosed herein, and/or using a sample that has been partitioned into a plurality of subsamples as disclosed herein. Unless otherwise indicated, where partitioning is performed on the basis of a cytosine modification, the first subsample is the subsample with a higher level of the modification; the second subsample is the subsample with a lower level of the modification; and, when present, the third subsample has a level of the modification intermediate between the first and second subsamples.

In some embodiments, methods herein comprise contacting DNA with a methylation-sensitive nuclease, thereby degrading DNA comprising unmethylated sequences or sequences having low levels of methylation. In some such embodiments, the methylation-sensitive nuclease is a methylation-sensitive restriction enzyme (MSRE), thereby degrading DNA comprising an unmethylated recognition site of the MSRE. Methylation-sensitive nucleases can thus be used in methods herein comprising one or more steps that deplete unmodified or unmethylated sequences, such as those that are prevalent in cfDNA from a subject.

In some embodiments, methods herein comprise contacting DNA with a methylation-dependent nuclease, thereby degrading DNA comprising methylated sequences or sequences having high levels of methylation. In some such embodiments, the methylation-dependent nuclease is a methylation-dependent restriction enzyme (MDRE), thereby degrading DNA comprising an methylated recognition site of the MSRE. Methylation-dependent nucleases can thus be used in methods herein comprising one or more steps that deplete modified or methylated sequences, such as those that are prevalent in cfDNA from a subject.

As discussed above, partitioning procedures may result in imperfect sorting of DNA molecules among the subsamples. The choice of a methylation-dependent nuclease or methylation-sensitive nuclease can be made so as to degrade nonspecifically partitioned DNA. For example, the second subsample can be contacted with a methylation-dependent nuclease, such as a methylation-dependent restriction enzyme. This can degrade nonspecifically partitioned DNA in the second subsample (e.g., methylated DNA) to produce a treated second subsample. Alternatively or in addition, the first subsample can be contacted with a methylation-sensitive endonuclease, such as a methylation-sensitive restriction enzyme, thereby degrading nonspecifically partitioned DNA in the first subsample to produce a treated first subsample. Degradation of nonspecifically partitioned DNA in either or both of the first or second subsamples is proposed as an improvement to the performance of methods that rely on accurate partitioning of DNA on the basis of a cytosine modification, e.g., to detect the presence of aberrantly modified DNA in a sample, to determine the tissue of origin of DNA, and/or to determine whether a subject has cancer. For example, such degradation may provide improved sensitivity and/or simplify downstream analyses. In general, where nonspecifically partitioned DNA would be hypermethylated, such as in a hypomethylated partition, a methylation-dependent nuclease, such as a methylation-dependent restriction enzyme, should be used. Conversely, where nonspecifically partitioned DNA would be hypomethylated, such as in a hypermethylated partition, a methylation-sensitive nuclease, such as a methylation-sensitive restriction enzyme, should be used. Methylation-dependent nucleases, such as methylation-dependent restriction enzymes, preferentially cut methylated DNA relative to unmethylated DNA, while methylation-sensitive nucleases, such as methylation-sensitive restriction enzymes, preferentially cut unmethylated DNA relative to methylated DNA.

In a contacting a subsample with a nuclease, one or more nucleases can be used. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting the second subsample with more than one methylation-dependent restriction enzyme can more completely degrade nonspecifically partitioned hypermethylated DNA. Similarly, contacting the first subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used. In some embodiments, the methylation-dependent nuclease comprises FspEI. In some embodiments, the methylation-dependent nuclease comprises FspEI and MspJI, e.g., used sequentially.

In some embodiments, a methylation-sensitive nuclease comprises one or more of AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, FspEI is used for digesting the nucleic acid molecules in at least one subsample (e.g., a hypomethylated partition). In some embodiments, BstUI, HpaII and Hin6I are used for digesting the nucleic acid molecules in at least one subsample (e.g., a hypermethylated partition) and FspEI is used for digesting the nucleic acid molecules in at least one other subsample (e.g., a hypomethylated partition). In embodiments involving an intermediately methylated partition, the nucleic acid molecules therein may be digested with a methylation-sensitive nuclease or a methylation-dependent nuclease. In some embodiments, the nucleic acid molecules in an intermediately methylated partition are digested with the same nuclease(s) as the hypermethylated partition. For example, the intermediately methylated partition may be pooled with the hypermethylated partition and then the pooled partitions may be subjected to digestion. In some embodiments, the nucleic acid molecules in an intermediately methylated partition are digested with the same nuclease(s) as the hypomethylated partition. For example, the intermediately methylated partition may be pooled with the hypomethylated partition and then the pooled partitions may be subjected to digestion.

In some embodiments, a subsample is contacted with a nuclease as described above after a step of tagging or attaching adapters to both ends of the DNA. The tags or adapters can be resistant to cleavage by the nuclease using any of the approaches described above. In this approach, cleavage can prevent the nonspecifically partitioned molecule from being carried through the analysis because the cleavage products lack tags or adapters at both ends.

Alternatively, a step of tagging or attaching adapters can be performed after cleavage with a nuclease as described above. Cleaved molecules can be then identified in sequence reads based on having an end (point of attachment to tag or adapter) corresponding to a nuclease recognition site. Processing the molecules in this way can also allow the acquisition of information from the cleaved molecule, e.g., observation of somatic mutations. When tagging or attaching adapters after contacting the subsample with a nuclease, and low molecular weight DNA such as cfDNA is being analyzed, it may be desirable to remove high molecular weight DNA (such as contaminating genomic DNA) from the sample before the contacting step. It may also be desirable to use nucleases that can be heat-inactivated at a relatively low temperature (e.g., 65° C. or less, or 60° C. or less) to avoid denaturing DNA, in that denaturation may interfere with subsequent ligation steps.

Where a sample is partitioned into three subsamples, including a third subsample containing intermediately methylated molecules, the third subsample is in some embodiments contacted with a methylation-sensitive nuclease. Such a step may have any of the features described elsewhere herein with respect to contacting steps, and may be performed before or after a step of tagging or attaching adapters as discussed above. In some embodiments, the first and third subsamples are combined before being contacted with a methylation-sensitive nuclease. Such a step may have any of the features described elsewhere herein with respect to contacting steps, and may be performed before or after a step of tagging or attaching adapters as discussed above. In some embodiments, the first and third subsamples are differentially tagged before being combined.

Alternatively, where a sample is partitioned into three subsamples, including a third subsample containing intermediately methylated molecules, the third subsample is in some embodiments contacted with a methylation-dependent nuclease. Such a step may have any of the features described elsewhere herein with respect to contacting steps, and may be performed before or after a step of tagging or attaching adapters as discussed above. In some embodiments, the second and third subsamples are combined before being contacted with a methylation-dependent nuclease. Such a step may have any of the features described elsewhere herein with respect to contacting steps, and may be performed before or after a step of tagging or attaching adapters as discussed above. In some embodiments, the second and third subsamples are differentially tagged before being combined.

In some embodiments, the DNA is purified after being contacted with the nuclease, e.g., using SPRI beads. Such purification may occur after heat inactivation of the nuclease. Alternatively, purification can be omitted; thus, for example, a subsequent step such as amplification can be performed on the subsample containing heat-inactivated nuclease. In another embodiment, the contacting step can occur in the presence of a purification reagent such as SPRI beads, e.g., to minimize losses associated with tube transfers. After cleavage and heat inactivation, the SPRI beads can be re-used for cleanup by adding molecular crowding reagents (e.g., PEG) and salt.

J. Partitioning the Sample into a Plurality of Subsamples

Disclosed methods herein comprise analyzing DNA in a sample. In such methods, different forms of DNA (e.g., hypermethylated and hypomethylated DNA) can be physically partitioned based on one or more characteristics of the DNA into a plurality of subsamples. This approach can be used to determine, for example, whether certain sequences are hypermethylated or hypomethylated. Such partitioning can be performed before or after a step of dividing the DNA into a plurality of subsamples. Thus, partitioning can be performed using a DNA sample that has not been divided, and/or using one or more subsamples of a DNA sample that has been divided as described herein. In the disclosed methods, partitioning is not considered a form of enriching for one or more sets of target regions of DNA.

In some embodiments, the partitioning comprises contacting the DNA with an agent that recognizes a modification associated with (e.g., in) the DNA. In some embodiments, the agent that recognizes the modification is an antibody. In some embodiments, the agent is immobilized on a solid support. In some embodiments, the partitioning comprises immunoprecipitation, e.g., using the antibody agent, such as an antibody, immobilized on solid support.

In some embodiments, the modification is methylation, and in some such embodiments, the partitioning comprises partitioning on the basis of methylation level. In some such embodiments, the agent is a methyl binding reagent. In some embodiments, the methyl binding reagent specifically recognizes 5-methylcytosine. In some such embodiments, the agent is a hydroxymethyl binding reagent. In some embodiments, the methyl binding reagent specifically recognizes 5-hydroxymethylcytosine, biotinylated 5-hydroxymethylcytosine, glucosylated 5-hydroxymethylcytosine, or sulfonylated 5-hydroxymethylcytosine. In some embodiments, the partitioning comprises partitioning on the basis of binding to a protein comprising contacting the sample comprising the DNA with a binding reagent specific for the protein. In some such embodiments, binding reagent specifically binds a methylated protein, an acetylated protein, such as a methylated or acetylated histone. In some embodiments, the binding reagent specifically binds an unmethylated or unacetylated protein epitope.

In some embodiments, the modification is hydroxymethylation, and in some such embodiments, the partitioning comprises partitioning on the basis of hydroxymethylation level. In some such embodiments, the agent is a hydroxymethyl binding reagent, such as an antibody. In some embodiments, the hydroxymethyl binding reagent (e.g., antibody) specifically recognizes 5-hydroxymethylcytosine (5-hmC). In some embodiments, a modification such as hydroxymethylation is labeled (e.g., biotinylated, glucosylated, or sulfonated) before being contacted with an agent that recognizes the labeled form of the modification. For example, 5-hmC can be enzymatically glucosylated and then partitioned based on binding to J-binding protein 1. Exemplary methods of labeling and/or partitioning 5-hmC are provided, e.g., in Song et al., Nat. Biotech. 29:68-72 (2010); Ko et al., Nature 468:839-843 (2010); and Robertson et al., Nucleic Acids Res. 39: e55 (2011).

Where immunoprecipitation is used and involves an antibody that recognizes single-stranded DNA, the DNA may be converted to double-stranded form by complementary strand synthesis before a subsequent step. Such synthesis may use an adapter as a primer binding site, or can use random priming.

In some embodiments, a sample comprising DNA is partitioned into a plurality of subsamples. In some embodiments, the plurality of partitioned subsamples comprises two subsamples, a first subsample and a second subsample. In some embodiments, the plurality of partitioned subsamples comprises three subsamples, a first subsample, second subsample, and third subsample. In some embodiments, the methods comprise a partitioning step that is performed (a) prior to the enriching for one or more sets of epigenetic and/or sequence-variable target regions of DNA from the DNA; (b) after the enriching for one or more sets of epigenetic and/or sequence-variable target regions of DNA from the DNA; (c) prior to the subjecting the DNA of the first subsample to a procedure that affects a first nucleobase of the DNA differently from a second nucleobase of the DNA; (d) after the subjecting the DNA of the first subsample to a procedure that affects a first nucleobase of the DNA differently from a second nucleobase of the DNA; or (c) any combination of (a)-(d).

In some embodiments comprising a third partitioned subsample, the third subsample comprises DNA associated with a modification in a greater proportion than it is associated with DNA in the second subsample and in a lesser proportion that it is associated with DNA in the first subsample.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable target regions are analyzed to determine whether they show differential methylation characteristic of tumor cells or cells of a type that does not normally contribute to the DNA sample being analyzed (such as cfDNA), and/or particular immune cell types.

In some instances, a heterogeneous nucleic acid sample is partitioned into two or more partitions (sub-samples; e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. After sequencing, analysis of reads can be performed on a partition-by-partition level, as well as a whole DNA population level. Tags are used to sort reads from different partitions. Analysis to detect genetic variants can be performed on a partition-by-partition level, as well as whole nucleic acid population level. For example, analysis can include in silico analysis to determine genetic variants, such as copy number variations (CNVs), single nucleotide variations (SNVs), insertions/deletions (indels), and/or fusions in nucleic acids in each partition. In some instances, in silico analysis can include analysis to determine epigenetic variation (one or more of methylation, chromatin structure, etc.). Analysis can include in silico using sequence information, genomic coordinates length, coverage, and/or copy number. For example, coverage of sequence reads can be used to determine nucleosome positioning in chromatin. Tags are used to sort reads from different partitions. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogenous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Exemplary partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as HpaII, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents or binding reagents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning is performed after contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed herein.

Examples of agents that recognize a modified nucleobase contemplated herein include, but are not limited to:

(a) MeCP2 and MBD2 are proteins that preferentially binds to 5-methyl-cytosine over unmodified cytosine.

(b) RPL26, PRP8 and the DNA mismatch repair protein MHS6 preferentially bind to 5-hydroxymethyl-cytosine over unmodified cytosine.

(c) FOXK1, FOXK2, FOXP1, FOXP4 and FOX13 preferably bind to 5-formyl-cytosine over unmodified cytosine (Iurlaro et al., Genome Biol. 14: R119 (2013)).

(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

In some cases, different procedures are applied to different partitions to determine different characteristics of the initial sample. The DNA of at least one partition is subjected to an end repair and modification sensitive sequencing procedure according to the methods of the disclosure described herein. In some embodiments at least one partition is not subjected to the end repair and modification sensitive sequencing procedure according to the methods of the disclosure described herein. In cases where the modification sensitive sequencing procedure comprises a conversion procedure, corresponding sequences from the converted and non-converted partitions can be compared to identify single nucleotides that have undergone conversion and therefore identify corresponding modified nucleosides in the initial sample.

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

K. Pooling DNA from at Least a First and Second Subsamples or Portions Thereof.

In some embodiments, such as following a partitioning step, the methods comprise preparing a pool comprising at least a portion of the DNA of the second subsample (also referred to as the hypomethylated partition) and at least a portion of the DNA of the first subsample (also referred to as the hypermethylated partition). Target regions, e.g., including epigenetic target regions and/or sequence-variable target regions, may be enriched from the pool. The steps of enriching for a target region set from at least a portion of a subsample described elsewhere herein encompass enrichment steps performed on a pool comprising DNA from the first and second subsamples. A step of amplifying DNA in the pool may be performed before enriching for target regions from the pool. The enriching step may have any of the features described elsewhere herein.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target-regions from the hypermethylated and hypomethylated partitions and epigenetic target regions from the hypermethylated partition. As such, in methods where sequence-variable target-regions and epigenetic target regions are being enriched, the latter may be enriched to a lesser extent than one or more of the sequence-variable target-regions from the hypermethylated and hypomethylated partitions and epigenetic target regions from the hypermethylated partition. For example, sequence-variable target regions can be enriched from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of the hypomethylated partition, e.g., less than about 50% of the DNA of the hypomethylated partition, such as less than or equal to about 45%, 40%, 35%, 30%, 25%, 20%, 15%, 10%, or 5% of the DNA of the hypomethylated partition. In some embodiments, the pool comprises about 5%-25% of the DNA of the hypomethylated partition. In some embodiments, the pool comprises about 10%-20% of the DNA of the hypomethylated partition. In some embodiments, the pool comprises about 10% of the DNA of the hypomethylated partition. In some embodiments, the pool comprises about 15% of the DNA of the hypomethylated partition. In some embodiments, the pool comprises about 20% of the DNA of the hypomethylated partition.

In some embodiments, the pool comprises a portion of the hypermethylated partition, which may be at least about 50% of the DNA of the hypermethylated partition. For example, the pool may comprise at least about 55%, 60%, 65%, 70%, 75%, 80%, 85%, 90%, or 95% of the DNA of the hypermethylated partition. In some embodiments, the pool comprises 50-55%, 55-60%, 60-65%, 65-70%, 70-75%, 75-80%, 80-85%, 85-90%, 90-95%, or 95-100% of the DNA of the hypermethylated partition. In some embodiments, the second pool comprises all or substantially all of the hypermethylated partition.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise enriching for at least a first set of target regions from the first pool, e.g., wherein the first pool is as set forth in any of the embodiments above. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this enrichment step. In some embodiments, enriching for the first set of target regions from the first pool comprises contacting the DNA of the first pool with a first set of target-specific probes. In some embodiments, the first set of target-specific probes comprises target-binding probes specific for the sequence-variable target regions. In some embodiments, the first set of target-specific probes comprises target-binding probes specific for the sequence-variable target regions, hypomethylation variable target regions and/or fragmentation variable target regions.

In some embodiments, the methods comprise enriching for a second set of target regions or plurality of sets of target regions from the second pool, e.g., wherein the first pool is as set forth in any of the embodiments above. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this enrichment step. In some embodiments, enriching for the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is enriched from the second pool but not from the first pool. In some embodiments, a plurality of hypermethylation variable target regions are enriched from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

### L. Providing a Combined Subsample

Some embodiments of the present disclosure comprise combining DNA of at least a first and second subsample, wherein the at least first and second subsample were divided from a DNA sample. As described herein, dividing a DNA sample can comprise dividing the DNA into a plurality of subsamples can comprise physically separating the DNA sample into two, three, four, five, six, seven, eight, nine, ten, or more than ten subsamples. In some embodiments, at least a portion of the DNA of any combination of the two, three, four, five, six, seven, eight, nine, ten, or more than ten subsamples can be combined prior to sequencing, providing a combined subsample. In some embodiments, the step of combining comprises physically combining, such as within the same container, at least a portion of the DNA of a first subsample, at least a portion of the DNA of a second subsample, at least a portion of the DNA of a third subsample, at least a portion of the DNA of a fourth subsample, at least a portion of the DNA of a fifth subsample, at least a portion of the DNA of a sixth subsample, at least a portion of the DNA of a seventh subsample, at least a portion of the DNA of an eighth subsample, at least a portion of the DNA of a ninth subsample, and/or at least a portion of the DNA of a tenth subsample, or any combination thereof. A container (such as, but not limited to, a pipette tip, tube, cuvette, multi-well plate, flow cell, or other container) can include any suitable container.

The DNA of the combined subsample can be sequenced, such as in the same sequencing reaction, such as within the same flow cell. In any embodiment, a first subsample that is partitioned, enriched, converted, and/or digested as described herein may be combined with a second subsample that is (a) not partitioned, enriched, converted, and/or digested, or (b) is partitioned, converted, and/or digested as described herein using the same or different types of partitioning, conversion, and/or digestion as the first subsample; thereby providing a combined subsample. For example, in some embodiments, prior to a sequencing step, enriched DNA of the first subsample and DNA of the second subsample are combined, wherein the DNA of the second subsample is not enriched for one or more sets of target regions of DNA; thereby providing a combined subsample. In certain embodiments, the DNA of the first subsample undergoes a conversion step, such as any of the conversion steps described elsewhere herein, and the converted DNA of the first subsample and DNA of the second subsample are combined, wherein the DNA of the second subsample is not converted; thereby providing a combined subsample. In some embodiments, the DNA of the first subsample undergoes a first conversion step, such as any of the conversion steps described elsewhere herein; the DNA of the second subsample undergoes a second conversion step, such as any of the conversion steps described elsewhere herein (e.g., wherein the second conversion step is different from the first conversion step), and the converted DNA of the first subsample and the converted DNA of the second subsample are combined, thereby providing a combined subsample. In further embodiments, the DNA of the first subsample undergoes partitioning (e.g., to obtain a hypermethylated partition) and/or treatment with a methylation-sensitive nuclease (e.g., a methylation sensitive restriction enzyme) and the partitioned and/or digested DNA of the first subsample is combined with the DNA of the second subsample, optionally wherein the DNA of the second subsample has undergone conversion, e.g., according to any of the conversion steps described elsewhere herein, further optionally wherein the DNA of the second subsample is not digested; thereby providing a combined subsample. In some embodiments, DNA of the first subsample is digested, e.g., with a methylation-sensitive nuclease (such as a methylation sensitive restriction enzyme) and DNA of the second subsample is digested, e.g., with a methylation-dependent nuclease (such as a methylation dependent restriction enzyme) and the digested DNA of the first subsample and the digested DNA of the second subsample are combined, thereby providing a combined subsample. In further embodiments, DNA of the first subsample undergoes partitioning (e.g., to obtain a hypermethylated partition) and the partitioned DNA of the first subsample (e.g., hypermethylated partition) is combined with the DNA of the second subsample, wherein the DNA of the second subsample is not partitioned; thereby providing a combined subsample. In some embodiments, DNA of the first subsample undergoes partitioning (e.g., to obtain a hypermethylated partition) and DNA of the second subsample undergoes partitioning (e.g., to obtain a hypomethylated partition) and partitioned DNA of the first subsample (e.g., hypermethylated partition) and partitioned DNA of the second subsample (e.g., hypomethylated partition) are combined, thereby providing a combined subsample. In some embodiments, DNA of the first subsample undergoes partitioning (e.g., to obtain a hypermethylated partition) and DNA of the second sample undergoes conversion, optionally wherein the partitioning comprises methylation-based partitioning and the converting comprises bisulfite conversion, and the partitioned DNA of the first subsample (e.g., hypermethylated partition) is combined with the converted DNA of the second subsample; thereby providing a combined subsample. In some embodiments, DNA of the first subsample is digested, e.g., with a methylation-sensitive nuclease (such as a methylation sensitive restriction enzyme) and DNA of the second subsample undergoes a conversion step, e.g., any of the conversion steps described elsewhere herein, such as bisulfite conversion, and the digested DNA of the first subsample and the converted DNA of the second subsample are combined; thereby providing a combined subsample. In certain embodiments, DNA of the first subsample undergoes partitioning (e.g., to obtain a hypermethylated partition) and digestion, e.g., with a methylation-sensitive nuclease (such as a methylation sensitive restriction enzyme), and DNA of the second sample undergoes a conversion step, e.g., any of the conversion steps described elsewhere herein, such as bisulfite conversion, and the partitioned and digested DNA of the first subsample and the converted DNA of the second subsample are combined, thereby providing a combined subsample. In some embodiments, at least one subsample of the combined subsample is not enriched for one or more sets of target regions of DNA (and may or may not be subjected to another treatment as described herein, such as a procedure that affects a first nucleobase in the DNA differently from a second nucleobase in the DNA). The DNA of such a subsample may be sequenced (as part of the combined subsample), for example, to assess one or more broad, genome-wide signatures (e.g., global hypomethylation, somatic variations, and/or fragmentomic signatures). Any of the exemplary combinations described above may further include at least a portion of the DNA from one or more additional subsamples, and/or DNA from an additional sample or source. Thus, the presently disclosed methods can enable multiplexed sequencing analyses. In general, prior to any combining step, the DNA of different subsamples may be differentially tagged, e.g., by attaching adapters to the DNA that comprise tags indicative of the subsample in which the DNA was present. Adapters may further comprise additional elements such as barcodes, as described elsewhere herein.

In some embodiments, the combined subsample comprises 80-100%, such as 80-95%, 85-100%, or 85-95% of DNA from the first subsample. In some embodiments, the combined subsample comprises 0%, 0.5-5%, 1-10%, 5-20%, 15-30%, 25-40%, 35-50%, 45-60%, 55-70%, 65-80%, 75-90%, or 85-100% of DNA from the first subsample. In some embodiments, the combined subsample comprises less than or equal to about 90%, 85%, 80%, 75%, 70%, 65%, 60%, 55%, 50%, 45%, 40%, 35%, 30%, 25%, 20%, 15%, 10%, 5%, or 1% of DNA from the first subsample. In some embodiments, the combined subsample comprises 0%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of DNA from the first subsample.

In some embodiments, the combined subsample comprises 0.5-20%, such as 0.5-15%, 5-20%, or 5-15% of DNA from the first subsample. In some embodiments, the combined subsample comprises 0.5-5%, 1-10%, 5-20%, 15-30%, 25-40%, 35-50%, 45-60%, 55-70%, 65-80%, 75-90%, or 85-100% of DNA from the second subsample. In some embodiments, the combined subsample comprises less than or equal to about 90%, 85%, 80%, 75%, 70%, 65%, 60%, 55%, 50%, 45%, 40%, 35%, 30%, 25%, 20%, 15%, 10%, 5%, or 1% of DNA from the second subsample. In some embodiments, the combined subsample comprises 0%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of DNA from the second subsample.

In some embodiments, the combined subsample comprises 0.5-5%, 1-10%, 5-20%, 15-30%, 25-40%, 35-50%, 45-60%, 55-70%, 65-80%, 75-90%, or 85-100% of DNA from a third subsample. In some embodiments, the combined subsample comprises less than or equal to about 90%, 85%, 80%, 75%, 70%, 65%, 60%, 55%, 50%, 45%, 40%, 35%, 30%, 25%, 20%, 15%, 10%, 5%, or 1% of DNA from the third subsample. In some embodiments, the combined subsample comprises 0%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of DNA from the third subsample.

In certain embodiments, the combined subsample comprises substantially all DNA from the first subsample. In certain embodiments, the combined subsample comprises substantially all DNA from the second subsample. In certain embodiments, the combined subsample comprises substantially all DNA from a third subsample.

In some embodiments, the DNA of at least the first and second subsamples is not combined prior to sequencing. In some embodiments, the DNA of the plurality of subsamples is not combined prior to sequencing.

### M. Adapter Ligation or Addition; Tagging

Double-stranded nucleic acids e.g., DNA molecules in a sample, and single stranded nucleic acid molecules converted to double stranded molecules, can be linked to adapters at either one end or both ends. In the methods of the disclosure, adapters can be ligated to sample nucleic acids prior to the partitioning and/or conversion steps. In some embodiments, adapters may be ligated to the sample nucleic acids after the partitioning and conversion steps, but before the step of amplifying the nucleic acids which have been subjected to partitioning and conversion steps.

In some embodiments, the DNA is made ligatable, e.g., by extending the end overhangs of the DNA molecules, and adding adenosine residues to the 3′ ends of fragments and phosphorylating 5′ end of each DNA fragment. Typically, double stranded molecules are blunt ended by treatment with a polymerase with a 5′-3′ polymerase and a 3′-5′ exonuclease (or proof reading function), in the presence of all four standard nucleotides. Klenow large fragment and T4 polymerase are examples of suitable polymerase.

The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the sample nucleic acid molecules and the adapters have blunt ends. In sticky-end ligation, typically, the sample nucleic acid molecules bear an “A” overhang and the adapters bear a “T” overhang.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample nucleic acid molecule. In some instances, two adapters can be ligated to a single sample nucleic acid molecule, with one adapter ligated to each end of the sample nucleic acid molecule.

Adapters can include nucleic acid primer binding sites to permit amplification of a sample nucleic acid molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given nucleic acid molecule. Adapters of the same or different sequence can be linked to the respective ends of a sample nucleic acid molecule. In some embodiments, adapters of the same or different sequence are linked to the respective ends of the nucleic acid molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a nucleic acid molecule to be analyzed. Other exemplary adapters include T-tailed, C-tailed or hairpin shaped adapters. For example, a hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times.

The adapters used in the methods of the present disclosure may comprise one or more known nucleosides wherein the base has a known methylation status, such as 5mC nucleic acid bases. When using adapters comprise 5mC, the adapters can be ligated to the sample nucleic acid molecules prior to the conversion procedure. Analyzing the sequence data corresponding to these known 5mC nucleic acid bases allows for the efficiency of the conversion procedure to be measured, which can be used as a quality control measure for the conversion procedure. In instances where two adapters are ligated to a sample nucleic acid (one at each end), either or both of the adapters may comprise one or more nucleosides with a known methylation status.

Typically the primer binding site(s), sequencing primer binding site(s), sample index(es) and/or molecular barcode(s), if present, do not comprise the nucleosides with a methylation status that change base pairing specificity as a result of the conversion procedure.

Preferably adapters (e.g., Y-shaped adapters) are ligated to the sample nucleic acids prior to the conversion and partitioning steps.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some such methods, first adapters are added to the 5′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids (e.g., DNA) are linked at both ends to Y-shaped adapters including primer binding sites and tags. In some such embodiments, the molecules (e.g., DNA) are amplified.

In some embodiments, the DNA molecules of the sample may be tagged with sample indexes and/or molecular barcodes (referred to generally as “tags”).

Tags can be molecules, such as nucleic acids, containing information that indicates a feature of the molecule with which the tag is associated. For example, DNA molecules can bear a sample tag or sample index (which distinguishes molecules in one sample from those in a different sample), a partition tag (which distinguishes molecules in one partition from those in a different partition) and/or a molecular tag/molecular barcode (which distinguishes different molecules from one another (in both unique and non-unique tagging scenarios)).

Tagging DNA molecules is a procedure in which a tag is attached to or associated with the DNA molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another in both unique and non-unique tagging scenarios). For methods that involve a partitioning step, a partition tag (which distinguishes molecules in one partition from those in a different partition) may be included. In some embodiments, adapters added to DNA molecules comprise tags. In some such embodiments, the tag comprises one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites. In some embodiments, adapters include the same primer binding site.

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, 20-50 different tags (e.g., barcodes) are ligated to both ends of target nucleic acids. For example 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some embodiments, two or more populations, samples, subsamples, or partitions are differentially tagged, such as partitioned subsamples and/or subsamples that are differentially degraded using one or more methylation-sensitive nucleases. Tags can be used to label the individual DNA populations so as to correlate the tag (or tags) with a specific population or partition. In some embodiments, a single tag can be used to label a specific population or partition. In some embodiments, multiple different tags can be used to label a specific population or partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11:0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g. by blunt-end ligation or sticky-end ligation), or overlap extension polymerase chain reaction (PCR), among other methods. Such adapters are ultimately joined to the sample DNA molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after any conversion procedure. In the case of molecular barcodes and/or sample indexes being introduced through amplification processes, the conversion step will occur before the molecular barcodes and/or sample indexes are introduced. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample DNA molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically tags are about 5 to 20 or 6 to 15 nucleotides in length. The tags may be linked to sample DNA molecules randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation as part of an adapter) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original DNA molecule in the sample, start and stop genomic positions corresponding to the sequence of the original DNA molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original DNA molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene).

In certain embodiments, the number of different tags used to uniquely identify a number of molecules, z, in a class can be between any of 2*2, 3*z, 4*z, 5*z, 6*z, 7*2, 8*z, 9*2, 10*2, 11*2, 12*2, 13*z, 14*2, 15*2, 16*2, 17*2, 18*2, 19*2, 20*z or 100*z (e.g., lower limit) and any of 100,000*2, 10,000*2, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes. For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described in, for example, U.S. patents application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments a format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after tagging and/or partitioning, the molecules may be pooled for sequencing in a single run. In some embodiments, a sample tag is added to the molecules, e.g., in a step subsequent to addition of other tags and pooling. Sample tags can facilitate pooling material generated from multiple samples for sequencing in a single sequencing run.

In some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules based on one or more characteristics, the final tagged molecules in the library may no longer possess that characteristic. For example, while single stranded DNA molecules may be partitioned and/or tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Tags providing molecular identifiers or bar codes can be incorporated into or otherwise joined to adapters by ligation, overlap extension PCR among other methods. Generally, assignment of unique or non-unique identifiers, or molecular barcodes in reactions follows methods and systems described by U.S. patent applications 20010053519, 20030152490, 20110160078, and U.S. Pat. Nos. 6,582,908 and 7,537,898.

Tags can be linked to sample nucleic acids randomly or non-randomly. In some embodiments, they are introduced at an expected ratio of unique identifiers to microwells. The microwell plate can have 96, 384, or 1536 microwells. In some cases, they are introduced at an expected ratio of unique tags to microwells. For example, the unique identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique identifiers are loaded per genome sample. In some embodiments, the unique identifiers may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique identifiers are loaded per genome sample. In some embodiments, the average number of unique identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique identifiers per genome sample.

In some embodiments, unique identifiers (e.g., tags) may be predetermined or random or semi-random sequence oligonucleotides. In other embodiments, a plurality of barcodes may be used such that barcodes are not necessarily unique to one another in the plurality. In this example, barcodes may be ligated to individual molecules such that the combination of the bar code and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length, or number of base pairs, of an individual sequence read may also be used to assign a unique identity to such a molecule. As described herein, fragments from a single strand of nucleic acid having been assigned a unique identity may thereby permit subsequent identification of fragments from the parent strand.

Polynucleotides in a sample can be tagged with a sufficient number of different tags so that there is a high probability (e.g., at least 90%, at least 95%, at least 98%, at least 99%, at least 99.9% or at least 99.99%) that all polynucleotides mapping to a particular genomic region bear a different identifying tag (molecules within the region are substantially uniquely tagged). The genomic region to which the polynucleotides map can be, for example, (1) the entire panel of genes being sequenced, (2) some portion of that panel, such as mapping within a single gene, exon or intron, (3) a single nucleotide coordinate (e.g., at least one nucleotide in the polynucleotide maps to the coordinate, for example, the start position, stop position, mid-point or anywhere between) or (4) a particular pair of start/stop (begin/end) nucleotide coordinates. The number of different identifiers (tag counts) necessary to substantially uniquely tag polynucleotides is a function of how many original polynucleotide molecules in the sample that map to the region. This, in turn, is a function of several factors. One factor is the total number of haploid genome equivalents included in the assay. Another factor is the average size of the polynucleotide molecules. Another factor is the distribution of the molecules across the region. This, in turn, can be a function of the cleavage pattern-one may expect cleavage to occur primarily between nucleosomes so that more polynucleotides map across a nucleosome location than between nucleosomes. Another factor is the distribution of barcodes in the pool and the ligation efficiency of individual barcodes, potentially causing differences in affective concentration of one barcode versus another. Another factor is the size of the region within which the molecules to be uniquely tagged are confined (e.g., same start/stop or same exon).

The identifier can be a single barcode attached to one end of a molecule, or two barcodes, each attached to different ends of the molecule. Attaching barcodes independently to both ends of a molecule increases by square the number of possible identifiers. In this case the number of different barcodes is selected such that the combination of barcodes on each end of a particular polynucleotide has a high probability of being unique with respect to other polynucleotides mapping to the same selected genomic region.

In certain embodiments, the number of different identifiers or barcode combinations (tag count) used can be at least any of 64, 100, 400, 900, 1400, 2500, 5625, 10,000, 14,400, 22,500 or 40,000 and no more than any of 90,000, 40,000, 22,500, 14,400 or 10,000. For example, the number of identifiers or barcode combinations can be between 64 and 90,000, between 400 and 22,500, 400 and 14,400 or between 900 and 14,400.

In a sample comprising fragmented genomic DNA, e.g., cell-free DNA (cfDNA), from a plurality of genomes, there is some likelihood that more than one polynucleotide from different genomes will have the same start and stop positions (“duplicates” or “cognates”). The probable number of duplicates beginning at any position is a function of the number of haploid genome equivalents in a sample and the distribution of fragment sizes. For example, cfDNA has a peak of fragments at about 160 nucleotides, and most of the fragments in this peak range from about 140 nucleotides to 180 nucleotides. Accordingly, cfDNA from a genome of about 3 billion bases (e.g., the human genome) may be comprised of almost 20 million (2×10⁷) polynucleotide fragments. A sample of about 30 ng DNA can contain about 10,000 haploid human genome equivalents. (Similarly, a sample of about 100 ng of DNA can contain about 30,000 haploid human genome equivalents.) A sample containing about 10,000 (10⁴) haploid genome equivalents of such DNA can have about 200 billion (2×10¹¹) individual polynucleotide molecules. It has been empirically determined that in a sample of about 10,000 haploid genome equivalents of human DNA, there are about 3 duplicate polynucleotides beginning at any given position. Thus, such a collection can contain a diversity of about 6×10¹⁰-8×10¹⁰ (about 60 billion-80 billion e.g., about 70 billion (7×10¹⁰)) differently sequenced polynucleotide molecules.

The probability of correctly identifying molecules is dependent on initial number of genome equivalents, the length distribution of sequenced molecules, sequence uniformity and number of tags. The number can be calculated using a Poisson distribution. When the tag count is equal to one, that is, equivalent to having no unique tags or not tagging. Table 4 below lists the probability of correctly identifying a molecule as unique assuming a typical cell-free size distribution as above.

TABLE 4

| | Tag % Correctly |
|---|---|
| Tag Count | uniquely identified |
| 1000 human haploid | |
| genome equivalents | |
| 1 | 96.9643 |
| 4 | 99.2290 |
| 9 | 99.6539 |
| 16 | 99.8064 |
| 25 | 99.8741 |
| 100 | 99.9685 |
| 3000 human haploid | |
| genome equivalents | |
| 1 | 91.7233 |
| 4 | 97.8178 |
| 9 | 99.0198 |
| 16 | 99.4424 |
| 25 | 99.6412 |
| 100 | 99.9107 |

In this case, upon sequencing the genomic DNA, it may not be possible to determine which sequence reads are derived from which parent molecules. This problem can be diminished by tagging parent molecules with a sufficient number of unique identifiers (e.g., the tag count) such that there is a likelihood that two duplicate molecules, i.e., molecules having the same start and stop positions, bear different unique identifiers so that sequence reads are traceable back to particular parent molecules. One approach to this problem is to uniquely tag every, or nearly every, different parent molecule in the sample. However, depending on the number of haploid gene equivalents and distribution of fragment sizes in the sample, this may require billions of different unique identifiers.

This method can be cumbersome and expensive. In some aspects, methods and compositions are provided herein in which a population of polynucleotides in a sample of fragmented genomic DNA is tagged with n different unique identifiers, wherein n is at least 2 and no more than 100,000*z, wherein z is a measure of central tendency (e.g., mean, median, mode) of an expected number of duplicate molecules having the same start and stop positions. In certain embodiments, n is at least any of 2*2, 3*z, 4*z, 5*z, 6*z, 7*z, 8*z, 9*2, 10*2, 11*2, 12*2, 13*2, 14*2, 15*z, 16*z, 17*z, 18*z, 19*2, 20*z or 100*z (e.g., lower limit). In other embodiments, n is no greater than 100,000*z, 10,000*2, 2000*2, 1000*2, 500*z or 100*z (e.g., upper limit). Thus, n can range between any combination of these lower and upper limits. In certain embodiments, n is between 100*z and 1000*2, 5*z and 15*z, between 8*z and 12*z, or about 10*z. For example, a haploid human genome equivalent has about 3 picograms of DNA. A sample of about 1 microgram of DNA contains about 300,000 haploid human genome equivalents. The number n can be between 15 and 45, between 24 and 36, between 64 and 2500, between 625 and 31,000, or about 900 and 4000. Improvements in sequencing can be achieved as long as at least some of the duplicate or cognate polynucleotides bear unique identifiers, that is, bear different tags. However, in certain embodiments, the number of tags used is selected so that there is at least a 95% chance that all duplicate molecules starting at any one position bear unique identifiers. For example, a sample comprising about 10,000 haploid human genome equivalents of cfDNA can be tagged with about 36 unique identifiers. The unique identifiers can comprise six unique DNA barcodes. Attached to both ends of a polynucleotide, 36 possible unique identifiers are produced. Samples tagged in such a way can be those with a range of about 10 ng to any of about 100 ng, about 1 μg, about 10 μg of fragmented polynucleotides, e.g., genomic DNA, e.g., cfDNA. In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. In some embodiments comprising barcode attachment, e.g., randomly, to both ends of a molecule, the combination of barcodes, together, constitutes a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene).

Accordingly, the present disclosure also provides compositions of tagged polynucleotides. The polynucleotides can comprise fragmented DNA, e.g., cfDNA. A set of polynucleotides in the composition that map to a mappable base position in a genome can be non-uniquely tagged, that is, the number of different identifiers can be at least at least 2 and fewer than the number of polynucleotides that map to the mappable base position. A composition of between about 10 ng to about 10 μg (e.g., any of about 10 ng-1 μg, about 10 ng-100 ng, about 100 ng-10 μg, about 100 ng-1 μg, about 1 μg-10 μg) can bear between any of 2, 5, 10, 50 or 100 to any of 100, 1000, 10,000 or 100,000 different identifiers. For example, between 5 and 100 or between 100 and 4000 different identifiers can be used to tag the polynucleotides in such a composition.

Events in which different molecules mapping to the same coordinate (in this case having the same start/stop positions) and bearing the same, rather than different, tags, are referred to as “molecular collisions”. In certain instances, the actual number of molecular collisions may be greater than the number of theoretical collisions, calculated, e.g., as above. This may be a function of uneven distribution of molecules across coordinates, differences in efficiency of ligation between barcodes, and other factors. In this case, empirical methods can be used to determine the number of barcodes needed to approach the theoretical collision number. In one embodiment, provided herein is a method of determining a number of barcodes required to diminish barcode collisions for a given haploid genome equivalent based on length distribution of sequenced molecules and sequence uniformity. The method comprising creating a plurality of pools of nucleic acid molecules; tagging each pool with incrementally increasing numbers of barcodes; and determining an optimal number of barcodes that reduces the number of barcode collisions to a theoretical level, e.g., that could be due to differences in affective barcode concentrations due to differences is pooling and ligation efficiency.

In one embodiment, the number of identifiers necessary to substantially uniquely tag polynucleotides mapping to a region can be determined empirically. For example, a selected number of different identifiers can be attached to molecules in a sample, and the number of different identifiers for molecules mapping to the region can be counted. If an insufficient number of identifiers is used, some polynucleotides mapping to the region will bear the same identifier. In that case, the number identifiers counted will be less than the number of original molecules in the sample. The number of different identifiers used can be iteratively increased for a sample type until no additional identifiers, representing new original molecules, are detected. For example, in a first iteration, five different identifiers may be counted, representing at least five different original molecules. In a second iteration, using more barcodes, seven different identifiers are counted, representing at least seven different original molecules. In a third iteration, using more barcodes, 10 different identifiers are counted, representing at least ten different original molecules. In a fourth iteration, using more barcodes, 10 different identifiers, again, are counted. At this point, adding more barcodes is not likely to increase the number of original molecules detected.

### N. Detecting; Sequencing

In general, sample nucleic acids flanked by adapters with or without prior amplification can be subject to sequencing. Sequencing methods include, for example, Sanger sequencing, high-throughput sequencing, pyrosequencing, sequencing-by-synthesis, single-molecule sequencing (also known as single-molecule sequencing or third generation sequencing), nanopore sequencing (a type of long-read sequencing), 5-letter sequencing or 6-letter sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

The sequencing reactions can be performed on one more fragments types known to contain markers of cancer of other disease. The sequencing reactions can also be performed on any nucleic acid fragments present in the sample. The sequence reactions may provide for sequence coverage of the genome of at least 5%, 10%, 15%, 20%, 25%, 30%, 40%, 50%, 60%, 70%, 80%, 90%, 95%, 99%, 99.9% or 100%. In other embodiments, sequence coverage of the genome may be less than 5%, 10%, 15%, 20%, 25%, 30%, 40%, 50%, 60%, 70%, 80%, 90%, 95%, 99%, 99.9% or 100%.

In some embodiments, detection of the presence or absence of nucleic acid sequences and/or modifications comprises sequencing. In some embodiments, the nucleic acid are sequenced in a manner that distinguishes a first nucleobase from a second nucleobase.

Simultaneous sequencing reactions may be performed using multiplex sequencing. In some embodiments, cell-free nucleic acids may be sequenced with at least 1000, 2000, 3000, 4000, 5000, 6000, 7000, 8000, 9000, 10000, 50000, 100,000 sequencing reactions. In other embodiments, cell-free polynucleotides may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some embodiments, data analysis may be performed on at least 1000, 2000, 3000, 4000, 5000, 6000, 7000, 8000, 9000, 10000, 50000, 100,000 sequencing reactions. In other embodiments, data analysis may be performed on less than 1000, 2000, 3000, 4000, 5000, 6000, 7000, 8000, 9000, 10000, 50000, 100,000 sequencing reactions. An exemplary read depth is 1000-50000 or 1000-10000 or 1000-20000 reads per locus (base).

The sequencing method can be massively parallel sequencing, that is, simultaneously (or in rapid succession) sequencing any of at least 100, 1000, 10,000, 100,000, 1 million, 10 million, 100 million, or 1 billion nucleic acid molecules.

In some embodiments, detection of the presence or absence of DNA sequences and/or modifications comprises sequencing. In some embodiments, the DNA is sequenced in a manner that distinguishes a first nucleobase from a second nucleobase. In general, sample nucleic acids, including nucleic acids flanked by adapters, with or without prior amplification can be subject to sequencing. In general, sample nucleic acids flanked by adapters with or without prior amplification can be subject to sequencing.

Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously. For example, long-read sequencing (also referred to herein as single-molecule sequencing or third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine (Weirather J L, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “ID” reads (“template” and “complement”) by removing the adaptor. The consensus sequence of two “ID” reads is a “2D” read with a higher accuracy.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil- and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

Simultaneous sequencing reactions may be performed using multiplex sequencing. In some embodiments, cell-free nucleic acids may be sequenced with at least 1000, 2000, 3000, 4000, 5000, 6000, 7000, 8000, 9000, 10000, 50000, 100,000 sequencing reactions. In other embodiments cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%.

In some embodiments, the sequence reactions may provide for sequence coverage of, for example, at least 5%, 10%, 15%, 20%, 25%, 30%, 40%, 50%, 60%, 70%, or 80% of the genome. Sequence coverage can performed on, for example, at least 5, 10, 20, 70, 100, 200 or 500 different genes, or up to, for example, 5000, 2500, 1000, 500 or 100 different genes.

In general, sequencing of epigenetic target regions, e.g., to analyze a modified nucleoside profile of DNA, requires a lesser depth of sequencing than sequencing of a sequence-variable target region, e.g., for analysis of mutations. Hence, lesser sequencing depths, as described herein, may in some cases be adequate for the methods described herein.

In some embodiments, the sequencing comprises targeted sequencing in which one or more genomic regions of interest are sequenced. In some such embodiments, the genomic regions of interest comprise regions present one or more genes selected from Tables 3, 6, 7, 8, and/or 9. In some such embodiments, DNA sequences that do not comprise regions of interest are not sequenced. Some embodiments comprise non-targeted sequencing, e.g., all genomic regions of the DNA in a treated sample or subsample are sequenced, or genomic regions are randomly chosen for sequencing. In other embodiments, detecting DNA the presence or absence of sequences comprises sequencing DNA that is not enriched for genomic regions of interest (non-targeted sequencing), e.g., wherein detectable sequences are obtained in a substantially unbiased manner.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or at most 5000, 2500, 1000, 500 or 100 different genes.

Simultaneous sequencing reactions may be performed using multiplex sequencing. In some embodiments, cell-free nucleic acids may be sequenced with at least 1000, 2000, 3000, 4000, 5000, 6000, 7000, 8000, 9000, 10000, 50000, 100,000 sequencing reactions. In other embodiments cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

In some embodiments, sequences that were not cleaved during the degrading step are sequenced. In some embodiments, less than 50%, 40%, 30%, 20%, 10%, 5%, 4%, 3%, 2%, or 1% of sequences that were cleaved during the degrading step are sequenced.

### 1. Modification Sensitive Sequencing

The methods disclosed herein may use modification sensitive sequencing to detect the modification status of one or more nucleotides. This may include nucleotides present in the original sample and/or at least one type of dNTP comprising a modified base (such as mCTP) used in the end repair reaction. In some embodiments, a DNA sample comprising a plurality of DNA molecules is subjected to modification-sensitive sequencing to obtain sequencing data derived from the DNA sample, wherein the modification-sensitive sequencing comprises subjecting the plurality of DNA molecules to a procedure that affects a first nucleobase of the plurality of DNA molecules differently from a second nucleobase of the plurality of DNA molecules, wherein the first nucleobase is a modified or unmodified nucleobase, the second nucleobase is a modified or unmodified nucleobase different from the first nucleobase, and the first nucleobase and the second nucleobase have the same base pairing specificity, thereby producing a plurality of converted DNA molecules comprising one or more inappropriately converted bases and/or one or more inappropriately unconverted bases at one or more locations. Such embodiments may also comprise a step of end-repair prior to the modification-sensitive sequencing.

“Modification sensitive sequencing” refers to any sequencing workflow which is capable of distinguishing at least two modification states of a nucleotide bases. These two states may be: (i) whether a base is modified or not (e.g., 5mC and/or 5hmC vs unmethylated cytosine); or (ii) the type of modification which a base exhibits (e.g., 5mC vs 5hmC). Modification sensitive sequencing does not necessarily require that a specific type of modification is identified as present or absent at a specific position, just whether one or more modification types (e.g., 5mC and 5hmC) is present or absent. For instance, in some embodiments, modification sensitive sequencing includes sequencing comprising a bisulfite conversion step which can distinguish 5mC and 5hmC from unmethylated C, but it cannot distinguish between 5mC and 5hmC. In some embodiments, the modification-sensitive sequencing comprises subjecting a DNA sample (such as a DNA sample from a subject) to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

The type of modification sensitive sequencing used will depend on the type of modified base(s) used in the end repair, such that the type of modification sensitive sequencing will be able to detect at least the presence or absence of at least that modified base. Table 5 summarizes exemplary forms of modification sensitive sequencing with the type of modified bases detectable with these methods. These are described in more detail below.

TABLE 5
Exemplary modification sensitive sequencing methods

| | Base |
|---|---|
| Sequencing method | modification detectable |
| Bisulfite conversion | 5mC and 5hmC distinguishable |
| | from C |
| Oxidative bisulfite conversion | 5mC distinguishable from 5hmC |
| | and C |
| Tet-assisted bisulfite (TAB) | 5hmC distinguishable from 5mC |
| conversion | and C |
| APOBEC-coupled epigenetic | 5hmC distinguishable from 5mC |
| (ACE) conversion | and C |
| Enzymatic methyl-seq (EM-seq). | 5mC and 5hmC distinguishable |
| | from C |
| Single-enzyme 5-methylctyosine | 5mC and 5hmC distinguishable |
| sequencing (SEM-seq) | from C |
| Tet-assisted pyridine borane | 5mC and 5hmC distinguishable |
| sequencing (TAPS) | from C |
| Nanopore-based sequencing | 4mC, 5mC, 5hmC, 6mA, BrdU, |
| | FldU, IdU and EdU. |
| Single-molecule real-time | 4mC, 5mC, 5hmC, 6mA, and |
| sequencing (SMRT) | 8oxoG. |

As outlined below, there are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing. Together, the conversion procedure and the sequencing itself constitutes one form of modification aware sequencing, as referred to herein.

In some cases, a conversion procedure used in the methods of the disclosure is one that changes the base pairing specificity of a modified nucleoside (e.g., methylated cytosine), but does not change the base pairing specificity of the corresponding unmodified nucleoside (e.g., cytosine) or does not change the base pairing specificity of any un-modified nucleoside (e.g., cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as TAPS may in some cases be preferred over methods such as bisulfite sequencing and EM-seq because they are less destructive (especially important for low yield samples such as cfDNA or FFPE samples) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) non-conversion events can appear as false negatives (non-methylated regions). Random non-conversion methods can maximally affect a low percent of bases within a region, and thus the specificity of methylation change detection can be maximized (reduce false positives) by placing a threshold on percentage of bases within a region that are methylated/non-methylated. Hence, in some cases, a conversion procedure that does not involve denaturation is preferred.

In other cases, a conversion procedure used in the methods of the disclosure is one that changes the base pairing specificity of an unmodified nucleoside (e.g., cytosine), but does not change the base pairing specificity of the corresponding modified nucleoside (e.g., methylated cytosine such as 5hmC and/or 5mC). Such methods include, for example, bisulfite sequencing.

The skilled person can select a suitable method according to their needs, including which nucleoside modifications are to be detected and/or identified and which type of modified base is used in the end repair reaction.

In some embodiments, the conversion procedure converts modified nucleosides. In some embodiments, the conversion procedure which converts modified nucleosides comprises Tet-assisted conversion with a substituted borane reducing agent, optionally wherein the substituted borane reducing agent is 2-picoline borane, borane pyridine, tert-butylamine borane, ammonia borane or pyridine borane. In Tet-assisted pic-borane conversion with a substituted borane reducing agent conversion, a TET protein is used to convert 5mC and 5hmC to 5caC, without affecting unmodified C. 5caC, and 5fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Sequencing of the converted DNA identifies positions that are read as cytosine as being unmodified C positions. Meanwhile, positions that are read as T are identified as being T, 5mC, 5fC, 5caC, or 5hmC. Performing TAP conversion, such as on a DNA sample as described herein, thus facilitates identifying positions containing unmodified C using the sequence reads obtained.

Hence, in these embodiments, the end repair reaction can be performed with dNTPs, wherein the at least one type of dNTP comprises a 5mC or 5hmC, and regions synthesized during the end repair reaction can be identified as those regions comprising 5mC or 5hmC (via T being called at positions which are C in the reference) at non-CpG positions. This procedure encompasses Tet-assisted pyridine borane sequencing (TAPS), described in further detail in Liu et al. 2019, supra. In this method Tet enzyme is used to progressively oxidize 5mC and 5hmC to 5fC or 5caC, then pyridine borane deaminates 5fC, 5CaC to DHU, amplified as T.

Alternatively, protection of 5hmC (e.g., using BGT or 5-hydroxymethylcytosine carbamoyltransferase) can be combined with Tet-assisted conversion with a substituted borane reducing agent, e.g., as described above. In this method (TAPS-β), 5hmC can be protected from conversion, for example through glucosylation using β-glucosyl transferase (βGT), forming (forming 5-glucosylhydroxymethylcytosinc) 5ghmC, or through carbamoylation using 5-hydroxymethylcytosine carbamoyltransferase, forming 5cmC. A method of protecting 5hmC from conversion, for example through glucosylation using β-glucosyltransferase (βGT), forming 5-glucosylhydroxymethylcytosine (5ghmC), is described in Yu et al., Cell 2012; 149:1368-80. Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 or a TET2 comprising a T1372S mutation can be used to convert mC to caC, and then bisulfite treatment can be used to convert C and caC to U while 5cmC remains unaffected. In this method, 5hmC can be protected from conversion, for example through glucosylation using β-glucosyltransferase (βGT), forming 5-glucosylhydroxymethylcytosine (5ghmC). Treatment with a TET protein, such as mTet1 or a TET2 comprising a T1372S mutation, then converts 5mC to 5caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either 5hmC or unmodified C positions. Meanwhile, positions that are read as T are identified as being T, 5fC, 5caC, or 5mC. Performing TAPSβ conversion on a sample as described herein thus facilitates distinguishing positions containing unmodified C or 5hmC on the one hand from positions containing 5mC using the sequence reads obtained. Hence, in these embodiments, the end repair reaction can be performed with dNTPs, wherein the at least one type of dNTP comprises a 5mC, and regions synthesized during the end repair reaction can be identified as those regions comprising 5mC (via T being called at positions which are C in the reference) at non-CpG positions. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12 (17): e4496.

In some embodiments, the conversion procedure converts modified nucleosides. In some embodiments, the conversion procedure which converts modified nucleosides comprises chemical-assisted conversion with a substituted borane reducing agent, optionally wherein the substituted borane reducing agent is 2-picoline borane, borane pyridine, tert-butylamine borane, borane pyridine or ammonia borane. In chemical-assisted conversion with a substituted borane reducing agent, an oxidizing agent such as potassium perruthenate (KRuO₄) (also suitable for use in ox-BS conversion) is used to specifically oxidize 5hmC to 5fC. Treatment with pic-borane or another substituted borane reducing agent such as borane pyridine, tert-butylamine borane, or ammonia borane converts 5fC and 5caC to DHU but does not affect 5mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either 5mC or unmodified C positions. Meanwhile, positions that are read as T are identified as being T, 5fC, 5caC, or 5hmC. Performing this type of conversion as described herein thus facilitates distinguishing positions containing unmodified C or 5mC on the one hand from positions containing 5hmC using the sequence reads obtained. Hence, in these embodiments, the end repair reaction can be performed with dNTPs, wherein at least one type of dNTP comprises a 5hmC, and regions synthesized during the end repair reaction can be identified as those regions comprising 5hmC (via T being called at positions which are C in the reference) at non-CpG positions. For an exemplary description of this type of conversion, see, e.g., Liu et al., Nature Biotechnology 2019; 37:424-429.

Exemplary conversion procedures that change the base-pairing specificity of modified cytosines have been described. However, the methods described herein could in principle use any modified nucleoside and suitable conversion procedure (i.e. single-base epigenetic conversion assay) that changes the base-pairing specificity of the modified nucleoside and thereby allows the modified base to be distinguished from the corresponding unmodified nucleoside and/or other types of modification when sequenced. For example, any conversion procedure could be used allowing any one of N⁶-methyladenine (6 mA), N⁶-hydroxymethyladenine (6hmA), or N6-formyladenine (6fA) to be distinguished from unmodified adenosine.

In some embodiments, the conversion procedure converts unmodified nucleosides. In some embodiments, the conversion procedure which converts unmodified nucleosides comprises bisulfite conversion. Treatment with bisulfite converts unmodified cytosine and certain modified cytosine nucleotides (e.g., 5-formyl cytosine (5fC) or 5-carboxylcytosine (5caC)) to uracil whereas other modified cytosines (e.g., 5mC and 5hmC) are not converted. Thus, where bisulfite conversion is used, the first nucleobase comprises one or more of unmodified cytosine, 5fC, 5caC, or other cytosine forms affected by bisulfite, and the second nucleobase may comprise one or more of 5mC and 5hmC, such as 5mC and optionally 5hmC. Sequencing of bisulfite-treated DNA identifies positions that are read as cytosine as being 5mC or 5hmC positions. Meanwhile, positions that are read as T are identified as being T or a bisulfite-susceptible form of C, such as unmodified cytosine, 5fC, or 5caC. Thus, performing bisulfite conversion, such as on a DNA sample as described herein facilitates identifying positions containing 5mC or 5hmC. Hence, in these embodiments, the end repair reaction can be performed with dNTPs, wherein at least one type of dNTP comprises a 5mC and/or a 5hmC, and regions synthesized during the end repair reaction can be identified as those regions comprising 5mC or a 5hmC (via C being called at these positions) at non-CpG positions. For an exemplary description of bisulfite conversion, see, e.g., Moss et al., Nat Commun. 2018; 9:5068.

In some embodiments, the procedure which converts unmodified nucleosides comprises oxidative bisulfite (Ox-BS) conversion. This procedure first converts 5hmC to 5fC, which is bisulfite susceptible, followed by bisulfite conversion. Thus, when oxidative bisulfite conversion is used, the first nucleobase comprises one or more of unmodified cytosine, 5fC, 5caC, 5hmC, or other cytosine forms affected by bisulfite, and the second nucleobase comprises 5mC. Sequencing of Ox-BS converted DNA identifies positions that are read as cytosine as being 5mC positions. Meanwhile, positions that are read as T are identified as being T or a bisulfite-susceptible form of C, such as unmodified cytosine, 5fC, or 5hmC. Hence, in these embodiments, the end repair reaction can be performed with dNTPs, wherein at least one type of dNTP comprises a 5mC, and regions synthesized during the end repair reaction can be identified as those regions comprising 5mC (via C being called at these positions) at non-CpG positions. Performing Ox-BS conversion thus facilitates identifying positions containing mC. For an exemplary description of oxidative bisulfite conversion, see, e.g., Booth et al., Science 2012; 336:934-937.

In some embodiments, the procedure which converts unmodified nucleosides comprises Tet-assisted bisulfite (TAB) conversion. In TAB conversion, 5hmC is protected from conversion and 5mC is oxidized in advance of bisulfite treatment, so that positions originally occupied by 5mC are converted to U while positions originally occupied by 5hmC remain as a protected form of cytosine. For example, as described in Yu et al., Cell 2012; 149:1368-80, β-glucosyl transferase can be used to protect 5hmC (forming 5-glucosylhydroxymethylcytosine (5ghmC)), then a TET protein such as mTet1 can be used to convert 5mC to 5caC, and then bisulfite treatment can be used to convert C and 5caC to U while 5ghmC remains unaffected.

Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 can be used to convert mC to caC, and then bisulfite treatment can be used to convert C and caC to U while 5cmC remains unaffected. Thus, when TAB conversion is used, the first nucleobase comprises one or more of unmodified cytosine, 5fC, 5caC, 5mC, or other cytosine forms affected by bisulfite, and the second nucleobase comprises 5hmC. Sequencing of TAB-converted DNA identifies positions that are read as cytosine as being 5hmC positions. Meanwhile, positions that are read as T are identified as being T, or a bisulfite-susceptible form of C, such as unmodified cytosine, 5mC, 5fC, or 5caC. Performing TAB conversion on a first subsample as described herein thus facilitates identifying positions containing 5hmC. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions.

In some embodiments, the conversion procedure which converts unmodified nucleosides comprises APOBEC-coupled epigenetic (ACE) conversion. In ACE conversion, an AID/APOBEC family DNA deaminase enzyme such as APOBEC3A (A3A) is used to deaminate unmodified cytosine and 5mC without deaminating 5hmC, 5fC, or 5caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being 5hmC, 5fC, or 5caC positions. Meanwhile, positions that are read as T are identified as being T, unmodified C, or 5mC. Performing ACE conversion as described herein thus facilitates distinguishing positions containing 5hmC from positions containing 5mC or unmodified C using the sequence reads obtained from the first subsample. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12 (17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI having SEQ ID NO: 1 or SEQ ID NO: 2, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase of SEQ ID NO: 1 or SEQ ID NO: 2 can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (βGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149:1368-80, and in Yang et al., Bio-protocol, 2023; 12 (17): c4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, TET3, or a TET2 comprising a T1372S mutation. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate, as described elsewhere herein.

Modification sensitive sequencing also includes sequencing methods which do not rely on a conversion step, wherein the base pairing specificity of a base is changed dependent on its modification status. For instance, single molecule techniques such as nanopore based sequencing and single molecule real time sequencing can be used to directly detect modified bases.

For example, some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore. One example of a nanopore sequencing system is that commercialized by Oxford Nanopore Technologies (ONT). (See e.g., (Weirather et al., F1000Research, 6:100, 2017.) ONT sequencing directly sequences a native single-stranded DNA (ssDNA) molecule by measuring characteristic current changes as the bases are threaded through the nanopore by a molecular motor protein. ONT sequencing uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “ID” reads (“template” and “complement”) by removing the adaptor. The consensus sequence of two “ID” reads is a “2D” read with a higher accuracy.

Nanopore sequencing can be used to detect base modifications including 5mC, 5hmC, 6 mA, BrdU, FdU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the modification sensitive sequencing comprises nanopore sequencing. In such embodiments, the end repair may be performed using dNTPs, which comprise 4mC, 5mC, 5hmC, 6 mA, BrdU, FdU, IdU, and/or EdU.

Another modification sensitive single molecule sequencing technique is single molecule real time sequencing (SMRT) that has been commercialized by Pacific Biosciences. SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.)

Single molecule real time sequencing can be used to detect base modifications such as 4mC, 5mC, 5hmC, 6 mA, and 8oxoG (Gouil & Keniry Essays in Biochemistry (2019) 63 639-648). Accordingly, in some embodiments, the modification sensitive sequencing comprises single molecule real time sequencing. In such embodiments, the end repair may be performed using dNTPs, which comprise 4mC, 5mC, 5hmC, 6 mA, and/or 8oxoG.

### 2. Differential Depth of Sequencing

In some embodiments, nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

In some embodiments, the captured cfDNA corresponding to the hydroxymethylation variable target region set and the captured cfDNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the hydroxymethylation variable target region set and the captured cfDNA corresponding to the at least one other target region set in the same vessel.

### O. Analysis

Genetic data can be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure may be useful in determining disease progression.

The present methods are useful in determining the efficacy of a particular treatment option. The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing.

Further, the methods of the disclosure may be used to characterize the heterogeneity of an abnormal condition in a subject, the method comprising generating a genetic profile of extracellular polynucleotides in the subject, wherein the genetic profile comprises a plurality of data resulting from copy number variation and rare mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and rare mutation analyses alone or in combination.

The present methods can be used to diagnose, prognose, monitor or observe cancers or other diseases.

The present disclosure provides methods of analyzing DNA, such as a combined subsample of DNA as described herein. In some embodiments, the DNA of a plurality of subsamples is not combined prior to sequencing. In some embodiments, the disclosed methods comprise analyzing DNA (such as DNA from a subject) to identify at least one cell type, cell cluster type, tissue type, and/or cancer type from which one or more type-specific epigenetic target regions and/or type-specific sequence-variable target regions originated. In some embodiments, methods comprise determining the level of one or more type-specific epigenetic target regions and/or type-specific sequence-variable target regions that originated from the at least one cell type, cell cluster type, tissue type, and/or cancer type.

In some embodiments, detecting the presence, levels, or absence of DNA sequences and/or modifications facilitates disease diagnosis or identification of appropriate treatments. In some embodiments, the presence of or a change in the levels of one or more sequences and/or modifications is indicative of the presence or absence of a disease or disorder in a subject, such as cancer or precancer, or other disorder that causes changes in nucleic acids relative to a healthy subject.

Further, the present methods can be used to diagnose presence or absence of a condition, e.g., cancer or precancer, in a subject, to characterize a condition (e.g., staging cancer or determining heterogeneity of a cancer), to monitor response to treatment of a condition (such as a response to a chemotherapeutic or immunotherapeutic), to effect prognosis risk of developing a condition or subsequent course of a condition, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). In some embodiments, the condition is cancer or precancer. In some embodiments, the condition is characterized (e.g., staging cancer or determining heterogeneity of a cancer), response to treatment of a condition is monitored, or prognosis risk of developing a condition or subsequent course of a condition is determined. The present disclosure can also be useful in determining the efficacy of a particular treatment option. Successful treatment options may decrease the amount of detected DNA sequences associated with a cancer in a subject's blood as there may be fewer cancer cells to shed DNA. In other examples, this may not occur. In another example, certain treatment options may be correlated with genetic profiles of cancers over time. This correlation may be useful in selecting a therapy. In some embodiments, successful treatment options may result in increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in cfDNA or DNA isolated from a buffy coat sample or any other sample comprising cells, such as in a blood sample (e.g., a whole blood sample, a leukapheresis sample, or a PBMC sample) from the subject) if the treatment is successful as more cancer cells may die and shed DNA, or, e.g., if a successful treatment results in an increase or decrease in the quantity of a specific protein in the blood and an unsuccessful treatment results in no change. In some embodiments, successful treatment options may result in changes in levels of different immune cell types (including rare immune cell types). Additionally, if a cancer is observed to be in remission after treatment, the present methods can be used to monitor the likelihood of residual disease or the likelihood of recurrence of disease.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, colon cancers, prostate cancers, thyroid cancers, bladder cancers, head and neck cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In some embodiments, a method described herein comprises detecting the presence or absence of nucleic acids, such as DNA, produced by a tumor (or neoplastic cells, or cancer cells) or by precancer cells.

Information and data generated by the methods disclosed herein can also be used for characterizing a specific form of cancer. Cancers are often heterogeneous in both composition and staging. The methods disclosed herein may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of a condition in a subject. Such methods can include, e.g., generating an aggregate profile of extracellular nucleic acids derived from the subject, wherein the aggregate profile comprises a plurality of data resulting from various nucleic acid analyses. In some embodiments, the aggregate profile comprises epigenetic and mutation analyses. In some embodiments, an aggregate profile comprises a summation of information derived from different cells in a heterogeneous disease. This summation may comprise structural variation identities and levels, copy number variation, epigenetic variation, or other mutation analyses.

The present methods can be used to diagnose, prognose, monitor or observe pre-cancers, cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### P. End Repair and A-Tailing

End repair refers to methods for repairing DNA by the conversion of non-blunt ended DNA into blunt ended DNA. Sequencing workflows typically use end repair to make ends of DNA molecules compatible with adapters, which are subsequently ligated onto the DNA. Fragmented and/or damaged DNA (e.g., cfDNA or DNA from FFPE samples) often contain non-blunt ends, which contain 3′overhangs and/or 5′overhangs. A 3′overhang refers to the 3′ end of a DNA strand which extends beyond 5′ end of the paired strand, resulting in one or more unpaired nucleotides at 3′ end of the DNA strand. Conversely, a 5′overhang refers to 5′ end of a DNA strand which extends beyond 3′ end of the paired strand, resulting in one or more unpaired nucleotides at 5′ end of the DNA strand.

The process of end repair involves the conversion of double-stranded DNA with 3′overhangs and/or 5′overhangs to double-stranded DNA without overhangs. This can be done using one or more enzymes such as T4 DNA polymerase and/or Klenow fragment. The 3′ to 5′ exonuclease activity of these enzymes removes 3′ ends at 3′overhangs and 5′ to 3′ polymerase activity of these enzymes extends 3′ ends at 5′ overhangs to remove 5′ overhang, thereby generating a blunt-ended DNA molecule. In order to fill in these 5′ overhangs, end repair is conducted in the presence of dATP, dCTP, dGTP and dTTP. End repair can also include a second step, which involves the addition of a phosphate group to 5′ ends of DNA, by an enzyme such as polynucleotide kinase. This makes 5′ ends of the end-repaired DNA molecules compatible with the subsequent action of DNA polymerases and DNA ligases.

As used herein, the term “A-tailing” refers to the addition of a single deoxyadenosine residue to the end of a blunt-ended double-stranded DNA fragment to form a 3′ deoxyadenosine single-base overhang. Such A tailing reactions are conducted with polymerases which have the ability to add a non-templated A to the 3′ end of a blunt, double-stranded DNA molecule. Polymerases capable of A-tailing typically do not possess 3′-5′ exonuclease activity. When A-tailing is performed as a separate reaction to end repair, it is typically conducted in the presence of dATP, but the absence of dCTP, dTTP and dGTP. A-tailed fragments are not compatible for self-ligation (i.e., self-circularization and concantenation of the DNA), but they are compatible with 3′ T-overhangs, which can be used on adapters. Methods comprising end repair, A-tailing and ligation to adapters with 3′ T-overhangs can result in higher efficiency ligation, compared to blunt ended ligation, as blunt ligation can lead to self-ligation of the adapters and/or DNA molecules.

In some embodiments, the methods disclosed herein comprise end repair of the DNA molecules followed by blunt end ligation of adapters. In other embodiments, the methods disclosed herein comprise end repair of the DNA molecules followed by A-tailing and sticky-end ligation of T-tailed adapters. When the methods disclosed herein comprise an A-tailing step, it may be performed separately from the end repair with an intervening reaction clean-up step or it may be performed in the same reaction as the end repair (e.g., using NEBNext® Ultra™ II End Repair/dA-Tailing Module (E7546)). In instances wherein the A-tailing reaction is performed in the same reaction as end repair, the sticky-end ligation may be performed with a mixture of T-tailed adapters and C-tailed adapters.

In some embodiments, the end-repair and the A-tailing reactions are performed in a single tube. In such cases, the A tailing reaction can be performed at a higher temperature than the end repair. Optionally, end repair is performed at ambient temperature (e.g., 15-35° C.) and A tailing is performed at a temperature over 60° C. The A tailing reaction can be performed using a thermostabile polymerase (e.g., Taq DNA polymerase, Tfl DNA polymerase, Bst DNA Polymerase, Large Fragment or Tth DNA polymerase) and the method further comprises increasing temperature of the sample after the end repair to inactivate the polymerase used in end repair (e.g., T4 DNA polymerase or Klenow fragment). In some embodiments the A-tailing is performed using a DNA polymerase that: (i) does not possess 5′-3′ exonuclease activity; and/or (ii) is not a strand displacing DNA polymerase. These properties reduce the ability of the DNA polymerase to extend from nick. This reduces the level of synthesis which may occur during the end repair and A-tailing reactions thus reducing the proportion of sequencing data that may be filtered out as potentially containing artifactual data. Accordingly, in some embodiments, the A-tailing is performed using a DNA polymerase that cannot extend from a nick in the DNA such as HemoKlen Taq. In other embodiments, the A-tailing is performed using Taq DNA polymerase. In other embodiments, the A-tailing is performed using Tfl polymerase, Bst DNA Polymerase, Large Fragment or Tth polymerase.

In some embodiments, the methods disclosed herein comprise an A tailing reaction after the end repair and before the ligation reaction, wherein the end repair and A tailing reactions are separated by a reaction cleanup. The A tailing reaction is typically performed in the presence of dATP, but in the absence of dCTP, dTTP and dGTP. In some embodiments, the A tailing reaction is performed using Klenow Fragment lacking 3′-5′ exonuclease activity.

In some embodiments, a dNTP that comprises a modified base is used in end repair, which may be any modified base wherein the presence or the absence of the modification can be detected by a type of modification sensitive sequencing. The modified base may be 4-methylcytosine (4mC), 5-methylcytosine (5mC), 5-hydroxymethyl-cytosine (5hmC), N6-methyladenosine (6 mA), bromodeoxyuridine (BrdU), 5-fluorodeoxyuridine (FldU), 5-iododeoxyuridine (IdU), 5-ethynyldeoxyuridine (EdU) and/or 8-oxoguanine (8oxoG).

When a dNTP comprising a modified base is used, it may be used in place of the equivalent unmodified base in the end repair reaction. For instance, if a dCTP comprising 5mC is used in the end repair reaction, there may be no dCTP comprising an unmodified cytosine. This would ensure that dCTPs incorporated into the DNA molecule during the end repair reaction contain 5mC. In some embodiments, multiple types of dNTP comprising a modified base are used in the end repair. For example, dATP comprising 6 mA and dCTP comprising 5mC can be used in the end repair reaction in place of dATP comprising unmodified adenine and dCTP comprising unmodified cytosine. The use of multiple types of dNTP comprising a modified base is advantageous because it provides increased resolution in defining the regions of the end-repaired DNA molecule which have been synthesized during the end repair reaction. This is because, in this example, the end of a synthesized region can be defined as the first unmodified adenine or unmodified cytosine after a stretch of containing 6mAs and/or 5mCs, rather than relying on the detection of solely an unmodified adenine or solely an unmodified cytosine.

The modification sensitive sequencing method used will depend on the type of modified base used in the end-repair reaction such that the specific modification can be detected. Exemplary conversion-based methods are described above alongside the base modification which they can detect. Moreover, nanopore-based sequencing can be used to detect 4mC, 5mC, 5hmC, 6 mA, BrdU, FdU, IdU, and EdU, and single-molecule real time (SMRT) sequencing from Pacific Biosciences can be used to detect 4mC, 5mC, 5hmC, 6 mA, and 8oxoG.

### Q. Target Region Sets

In some embodiments, certain genomic regions of interest are detected and/or enriched. The genomic regions of interest may comprise one or more target region sets. In some embodiments, target region sets comprise variations that are not prevalent in DNA from healthy subjects or not prevalent in DNA obtained from healthy tissue regions. In some embodiments, target region sets comprise variations present in healthy cells but not normally present in the sample type, such as a blood sample. In some embodiments, the variations are present in aberrant cells (e.g., hyperplastic, metaplastic, or neoplastic cells). Exemplary target region sets include sequence-variable target region sets, epigenetic target region sets.

In some embodiments, a first target region set is detected, comprising at least epigenetic target regions. In some embodiments, the epigenetic target regions detected in a first subsample comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions show type-specific hypermethylation in healthy cfDNA from one or more related cell or tissue types. Without wishing to be bound by any particular theory, the presence of cancer cells may increase the shedding of DNA into the bloodstream (e.g., from the cancer and/or the surrounding tissue). As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, the methods herein comprise detecting a second captured target region set from a sample or second subsample, comprising at least epigenetic target regions. In some embodiments, the second epigenetic target region set comprises hypomethylation variable target regions. In some embodiments, the hypomethylation variable target regions are CpG-containing regions that are methylated or have high methylation in cfDNA from healthy subjects (e.g., above-average methylation relative to bulk cfDNA). Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

Additionally, target region sets may comprise DNA corresponding to a sequence-variable target region set.

### 1. Epigenetic Target Region Sets

In some embodiments, a target region set is or comprises an epigenetic target region set. Epigenetic target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation and Hypomethylation Variable Target Regions

In some embodiments, an epigenetic target region set comprises a hypermethylation variable target region. In some embodiments, the hypermethylation variable target regions are differentially or exclusively hypermethylated in one or more related cell or tissue types. Such hypermethylation variable target regions may be hypermethylated in other cell or tissue types but not to the extent observed in the one or more related cell or tissue types. In some embodiments, the hypermethylation variable target regions show even higher methylation in cfDNA from a diseased cell of the one or more related cell or tissue types.

In some embodiments, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In another example, as discussed above, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 6. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 6
Exemplary Hypermethylation Target
Regions based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, genomic regions targeted for sequencing comprise a plurality of loci listed in Table 6, e.g., at least 10%, 20%, 30%, 40%, 50%, 60%, 70%, 80%, 90%, or 100% of the loci listed in Table 6. In some embodiments, genomic regions are captured using probes. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 6, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5 (5): 492-504 (2016); Skvortsova et al., Br. J. Cancer. 94 (10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 3. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 6 and 3, indicating generality.

Any of the foregoing embodiments concerning target regions identified in Table 3 may be combined with any of the embodiments described above concerning target regions identified in Table 6. In some embodiments, genomic regions targeted for sequencing comprise a plurality of loci listed in Table 6 or Table 3, e.g., at least 10%, 20%, 30%, 40%, 50%, 60%, 70%, 80%, 90%, or 100% of the loci listed in Table 6 or Table 3.

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, an epigenetic target region set comprises a hypomethylation variable target region. In some embodiments, the hypomethylation variable target regions are exclusively hypomethylated in one or more related cell or tissue types. Such hypomethylation variable target regions may be hypomethylated in other cell or tissue types but not to the extent observed in the one or more related cell or tissue types.

In some embodiments, where different epigenetic target regions are captured, the epigenetic target regions comprise hypermethylation and/or hypomethylation variable target regions.

Further exemplary hypermethylation variable target regions and hypomethylation variable target regions useful for distinguishing between various cell types have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.cu.

b. CTCF Binding Regions

In some embodiments, an epigenetic target region set comprises CTCF binding regions. CTCF is a DNA-binding protein that contributes to chromatin organization and often colocalizes with cohesin. Perturbation of CTCF binding sites has been reported in a variety of different cancers. See, e.g., Katainen et al., Nature Genetics, doi: 10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Thus, perturbations of CTCF binding result in variation in the fragmentation patterns of cfDNA. As such, CTCF binding sites are a type of fragmentation variable target region.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

c. Transcription Start Sites

In some embodiments, an epigenetic target region set comprises variable transcription start sites. Transcription start sites may show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to DNA that is typical in healthy subjects. Perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34 (Database issue): D86-D89 (2006), which is incorporated herein by reference. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

d. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1.

e. Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### 2. Sequence-Variable Target Region Sets

In some embodiments, a target region set is or comprises a sequence-variable target region set. Sequence-variable target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The sequence-variable target region set may also comprise one or more control regions, e.g., as described herein. In some embodiments, a sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Examples of listings of genomic locations of interest may be found in, e.g., Table 7 and Table 8 herein. In some embodiments, a sequence-variable target region set comprises portions of at least 5, at least 10, at least 15, at least 20, at least 25, at least 30, at least 35, at least 40, at least 45, at least 50, at least 55, at least 60, at least 65, or 70 of the genes of Table 7. In some embodiments, a sequence-variable target region set comprises at least 5, at least 10, at least 15, at least 20, at least 25, at least 30, at least 35, at least 40, at least 45, at least 50, at least 55, at least 60, at least 65, or 70 of the SNVs of Table 7. In some embodiments, a sequence-variable target region set comprises portions of at least 1, at least 2, at least 3, at least 4, at least 5, or 6 of the fusions of Table 7. In some embodiments, a sequence-variable target region set comprises at least portions of at least 1, at least 2, or 3 of the indels of Table 7. In some embodiments, a sequence-variable target region set comprises portions of at least 5, at least 10, at least 15, at least 20, at least 25, at least 30, at least 35, at least 40, at least 45, at least 50, at least 55, at least 60, at least 65, at least 70, or 73 of the genes of Table 8. In some embodiments, a sequence-variable target region set comprises at least 5, at least 10, at least 15, at least 20, at least 25, at least 30, at least 35, at least 40, at least 45, at least 50, at least 55, at least 60, at least 65, at least 70, or 73 of the SNVs of Table 8. In some embodiments, a sequence-variable target region set comprises portions of at least 1, at least 2, at least 3, at least 4, at least 5, or 6 of the fusions of Table 8. In some embodiments, a sequence-variable target region set comprises at least portions of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 8. Each of these genomic locations of interest may be identified as a backbone region or hot-spot region for a given panel. Table 9 shows an example listing of hot-spot genomic locations of interest. In some embodiments, a sequence-variable target region set comprises portions of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 9. Each hot-spot genomic region is listed with the associated gene, chromosome on which it resides, the start and stop position of the genome representing the gene's locus, the length of the gene's locus in base pairs, the exons covered by the gene, and the critical feature (e.g., type of mutation) of a given genomic region of interest.

TABLE 7

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 8

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 9

| | | Start | Stop | Length | Exons | Critical |
|---|---|---|---|---|---|---|
| Gene | Chromosome | Position | Position | (bp) | Covered | Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 17 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

Examples of listings of target regions of interest may also be found in WO 2020/160414, e.g., at Table 4. Additional examples include loci disclosed in Gale et al., PLOS One 13: e0194630 (2018), incorporated herein by reference, which describes a panel of 35 cancer-related gene targets: AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1. In some embodiments, the sequence-variable target region set comprises target regions from at least 10, 20, 30, or 35 cancer-related genes, such as the cancer-related genes listed herein and in WO 2020/160414.

In some embodiments, the sequence-variable target region set has a footprint of at least 50 kbp, e.g., at least 100 kbp, at least 200 kbp, at least 300 kbp, or at least 400 kbp. In some embodiments, the sequence-variable target region set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

### R. Collections of Target-Specific Probes

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the capture probes probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of capture probes are configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes and/or target-binding probes are configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes or target-specific probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39:8740-8751 (2011); Freier et al., Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the capture probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes and/or target-binding probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 6, e.g., at least 10%, 20%, 30%, 40%, 50%, 60%, 70%, 80%, 90%, or 100% of the loci listed in Table 6. In some embodiments, the probes specific for hypermethylation variable target regions comprise probes specific for a plurality of loci listed in Table 3, e.g., at least 10%, 20%, 30%, 40%, 50%, 60%, 70%, 80%, 90%, or 100% of the loci listed in Table 3. In some embodiments, the probes specific for hypermethylation variable target regions comprise probes specific for a plurality of loci listed in Table 6 or Table 3, e.g., at least 10%, 20%, 30%, 40%, 50%, 60%, 70%, 80%, 90%, or 100% of the loci listed in Table 6 or Table 3. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 7. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 7. In some embodiments, probes specific for the sequence-variable target region set comprise probes specific for at least 1, at least 2, at least 3, at least 4, at least 5, or 6 of the fusions of Table 7. In some embodiments, probes specific for the sequence-variable target region set comprise probes specific for at least a portion of at least 1, at least 2, or 3 of the indels of Table 7. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 8. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 8. In some embodiments, probes specific for the sequence-variable target region set comprise probes specific for at least 1, at least 2, at least 3, at least 4, at least 5, or 6 of the fusions of Table 8. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 8. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 9.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

### S. Computer Systems

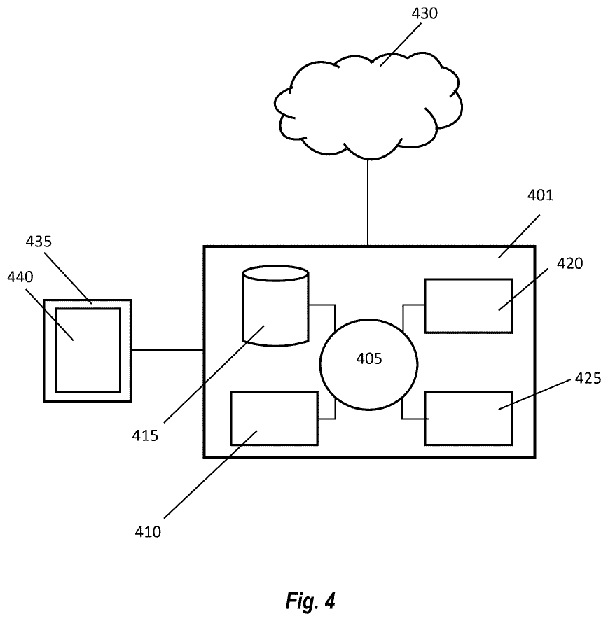

Methods of the present disclosure can be implemented using, or with the aid of, computer systems. FIG. 4 shows a computer system 401 that is programmed or otherwise configured to implement the methods of the present disclosure. The computer system 401 can regulate various aspects sample preparation, sequencing, and/or analysis. In some examples, the computer system 401 is configured to perform sample preparation and sample analysis, including (where applicable) nucleic acid sequencing, e.g., according to any of the methods disclosed herein.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage, and/or electronic display adapters. The memory 410, storage unit 415, interface 420, and peripheral devices 425 are in communication with the CPU 405 through a communication network or bus (solid lines), such as a motherboard. The storage unit 415 can be a data storage unit (or data repository) for storing data. The computer system 401 can be operatively coupled to a computer network 430 with the aid of the communication interface 420. The computer network 430 can be the Internet, an internet and/or extranet, or an intranet and/or extranet that is in communication with the Internet. The computer network 430 in some cases is a telecommunication and/or data network. The computer network 430 can include one or more computer servers, which can enable distributed computing, such as cloud computing. The computer network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. Examples of operations performed by the CPU 405 can include fetch, decode, execute, and writeback.

The storage unit 415 can store files, such as drivers, libraries, and saved programs. The storage unit 415 can store programs generated by users and recorded sessions, as well as output(s) associated with the programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet. Data may be transferred from one location to another using, for example, a communication network or physical data transfer (e.g., using a hard drive, thumb drive, or other data storage mechanism).

The computer system 401 can communicate with one or more remote computer systems through the network 430. For embodiment, the computer system 401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

In an aspect, the present disclosure provides a non-transitory computer-readable medium comprising computer-executable instructions which, when executed by at least one electronic processor, perform at least a portion of a method described herein. For example, see any of the embodiments set forth elsewhere herein, such as embodiments 1-105.

The code can be pre-compiled and configured for use with a machine have a processor adapted to execute the code or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### T. Applications

The methods disclosed herein may be used for monitoring the genetic, epigenetic, fragmentomic, and/or proteomic effects of a genome editing drug, including on-target and off-target edits and effects.

One important exemplary application of the methods of the disclosure is using the resulting sequencing data in diagnosing and prognosing cancer or other genetic diseases or conditions, e.g., that may arise as adverse effects of a genome-editing drug.

Hence, in some embodiments, methods described herein comprise identifying or predicting the presence or absence of DNA produced by a tumor (or neoplastic cells, or cancer cells), determining the probability that a test subject has a tumor or cancer, and/or characterizing a tumor, neoplastic cells or cancer as described herein.

### 1. Cancer and Other Diseases; Cell Type Quantification

The present methods can be used to diagnose presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may result in changes in levels of different immune cell types (including rare immune cell types), and/or increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a leukapheresis sample, or a PBMC sample) from the subject) if the treatment is successful as more cancer cells may die and shed DNA, or, e.g., if a successful treatment results in an increase or decrease in the quantity of a specific protein in the blood and an unsuccessful treatment results in no change.

Additionally, if a cancer is observed to be in remission after treatment, the present methods can be used to monitor the likelihood of residual disease or the likelihood of recurrence of disease.

In some embodiments, the present methods are used for screening for a cancer, or in a method for screening cancer. For example, the sample can be a sample from a subject who has not been previously diagnosed with cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at www.dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardio facial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

Furthermore, in some embodiments, the one or more methods described in the present disclosure may be used to assist in the treatment of a type of cancer.

These methods provided herein provide a deeper understanding of the changes in DNA and proteins that cause cancer, allowing the identification of biomarkers and design of treatments that target these proteins. In some embodiments, the biomarker may include an epigenetic signature, such as a methylation state, methylation score and/or DNA fragmentation pattern/score. In some embodiments, the epigenetic signature can be determined for one or more regions that include, but not limited to, transcription start sites, promoter regions, CTCF binding regions and regulatory protein binding regions. In some embodiments, the epigenetic signature is determined for one or more regions that include, but not limited to, transcription start sites, promoter regions, intergenic regions and/or intronic regions that are associated with at least one or more genes listed in any one or more of Tables 3, 6, 7, 8, and/or 9. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells.

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Subject as being a Candidate for a Subsequent Cancer Treatment

In some embodiments, a method provided herein is a method of determining a risk of cancer recurrence in a subject. In some embodiments, a method provided herein is a method of classifying a subject as being a candidate for a subsequent cancer treatment.

Any of such methods may comprise collecting DNA (e.g., originating or derived from a tumor cell) from the subject diagnosed with the cancer at one or more preselected timepoints following one or more previous cancer treatments to the subject. The subject may be any of the subjects described herein. The DNA may be DNA, such as cfDNA, from a blood sample (e.g., a whole blood sample, a buffy coat sample, a leukapheresis sample, or a PBMC sample). The DNA may comprise DNA obtained from a tissue sample.

Any of such methods may comprise enriching for a plurality of sets of target regions from DNA from the subject, wherein the plurality of target region sets comprise a sequence-variable target region set, and/or an epigenetic target region set, whereby a captured set of DNA molecules is produced. The enriching step may be performed according to any of the embodiments described elsewhere herein.

In any of such methods, the previous cancer treatment may comprise surgery, administration of a therapeutic composition, and/or chemotherapy.

Any of such methods may comprise sequencing the enriched DNA molecules, whereby a set of sequence information is produced. The enriched DNA molecules of a sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as cfDNA, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, from a blood sample (e.g., a whole blood sample, a buffy coat sample, a leukapheresis sample, or a PBMC sample), and/or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving one or more treatments for a condition (such as a response to a genome editing drug and/or a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening), such as based on changes in levels of different immune cell types, including rare immune cell types, in samples collected from a subject over time. In some embodiments, monitoring comprises analysis of at least two samples collected from a subject at at least two different time points as described herein.

The methods according to the present disclosure can also be useful in predicting a subject's response to a particular treatment option. Successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur. In another example, certain treatment options may be correlated with profiles (e.g., of immune cell types, proteins, and/or genetic profiles) of cancers over time. This correlation may be useful in selecting a therapy for a subject.

The disclosed methods can include evaluating (such as quantifying) and/or interpreting a quantity of one or more different immune cell types (including rare immune cell types), of a DNA sequence (such as one or more CDR3 sequences), and/or a protein or proteins present in one or more samples, such as one or more samples comprising cells or a blood sample (e.g., a buffy coat sample, a whole blood sample, a leukapheresis sample, or a PBMC sample), collected from a subject at one or more timepoints in comparison to a selected baseline value or reference standard (or a selected set of baseline values or reference standards). A baseline value or reference standard may be a quantity of the one or more different immune cell types (including rare immune cell types), a quantity of a DNA sequence (such as one or more CDR3 sequences), and/or a quantity of the protein or proteins measured in one or more samples (such as an average quantity or range of quantities of the protein or proteins present in at least two samples) collected from the subject at one or more time points, such as prior to receiving a treatment, prior to diagnosis of a condition (such as a cancer), or as part of a preventative health monitoring program. A baseline value or reference standard may be a quantity of the one or more different immune cell types (including rare immune cell types), a quantity of a DNA sequence (such as one or more CDR3 sequences), and/or a quantity of the protein or proteins measured in one or more samples (such as an average quantity or range of quantities of the protein or proteins present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a sample comprising cells or a blood sample (e.g., a buffy coat sample, a whole blood sample, a leukapheresis sample, or a PBMC sample) may be collected from a subject at two or more timepoints, to assess changes in the quantity of one or more immune cell types, the quantity of one or more DNA sequences (e.g., one or more CDR3 sequences), or the quantity of a protein or proteins (such as changes in quantities of the protein or proteins, or changes in one or more modifications (such as one or more post-translational modifications) of the protein or proteins) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring the quantity of one or more immune cell types, the quantity of one or more DNA sequences (e.g., one or more CDR3 sequences), or the quantity a protein or proteins and identifying differences between the immune cell types, DNA sequences, and/or protein or proteins in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein the quantity of one or more immune cell types, the quantity of one or more DNA sequences (e.g., one or more CDR3 sequences), or the quantity of a protein or proteins present in at least one sample (such as at least one whole blood sample, buffy coat sample, leukapheresis sample, or PBMC sample) collected from a subject at one or more timepoints (such as prior to receiving a treatment) is compared to the quantity of the one or more immune cell types, the quantity of the one or more DNA sequences (e.g., one or more CDR3 sequences), or the quantity of the protein or proteins present in at least one sample collected from the subject at one or more different time points (such as after receiving the treatment). The disclosed methods can allow for patient-specific monitoring, such that, for example, differences in immune cell type quantities, DNA sequence quantities (such as CDR3 sequence quantities, such as quantities of T-cell or B-cell specific CDR3 sequences), protein quantities and/or protein modifications between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples, such as a sample comprising cells or a blood sample (such as one or more whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples, such one or more samples comprising cells or one or more blood samples, e.g., one or more buffy coat samples, whole blood samples, leukapheresis samples, or PBMC samples, are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to patients or subjects, e.g., after identifying a condition in need of treatment that resulted from or arose following administration of a genome editing drug. In some embodiments, determination of the levels of particular immune cell types, including rare immune cell types, facilitates selection of appropriate treatment. In some embodiments, the patient or subject has a given disease, disorder or condition. In some embodiments of the disclosed methods, wherein the DNA and/or the sample is from a subject, the method further comprises determining the presence or status of a cancer in the subject. In some embodiments, determining the presence or status of a cancer in the subject can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject, e.g., at one or more timepoints, in comparison to a selected baseline value or reference standard (or a selected set of baseline values or reference standards), such as described in further detail below. In some such embodiments, the method further comprises administering at least one cancer therapy to a subject in need thereof (e.g., based on a determination of presence of a cancer in a subject). Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic agents such as immunotherapeutic agents can function by helping the immune system destroy cancer cells. For example, certain targeted therapeutic agents may mark cancer cells for the immune system to destroy them. Other targeted therapeutic agents may support the immune system to work more effectively against cancer. Yet other therapeutic agents may stop cancer cells from growing, for example, by interfering with cancer cell surface markers preventing them from dividing. Additionally, therapeutic agents can inhibit signals that promote angiogenesis. Such angiogenesis inhibitors prevent blood supply into the tumor thereby, preventing tumor growth. Other targeted therapeutic agents can deliver toxic substances to the tumor. Examples include monoclonal antibodies combined with toxins, chemotherapy, or radiation. Some targeted therapeutic agents induce apoptosis or deplete cancer of hormones.

In some embodiments, the therapeutic agents comprise one or more of PARP inhibitors, such as Olaparib (Lynparza), Rucaparib (Rubraca), Niraparib (Zejula), and Talazoparib (Talzenna). These may be used for treating mutations in BRCA1, BRCA2, ATM, BARD1, BRIP1, CDK12, CHEK1, CHEK2, FANCL, PALB2, RAD51B, RAD51 C, RAD51D and RAD54L alterations, and/or for genes associated Homologous Recombination Repair (HRR).

In some embodiments the treatment comprises one or more immunotherapies, immunotherapeutic agents and/or immune checkpoint inhibitors (ICIS). Immunotherapies are treatments with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1β, IL-6, and TNF-α. Other exemplary agents are T-cells activated against a tumor, such as T-cells activated by expressing a chimeric antigen targeting a tumor antigen recognized by the T-cell. In some embodiments, anti-PD-1 or anti-PD-L1 therapies comprise pembrolizumab (Keytruda), nivolumab (Opdivo), and cemiplimab (Libtayo), atezolizumab (Tecentriq), durvalumab (Imfinzi), and avelumab (Bavencio). These therapies may be used to treat patients identified as having high microsatellite instability (MSI) status or high tumor mutational burden (TMB).

In some embodiments, a therapeutic agent targets a mutated form of the EGFR protein. Such therapeutic agents can include osimertinib (Tagrisso), erlotinib (Tarceva), and gefinitib (Iressa).

Therapeutic agents can include one or more targeted therapeutic agents, including any one or more of abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Belcodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aliqopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azedra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride (Votrient), pembrolizumab (Keytruda), pemigatinib (Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

Table 10 provides an exemplary list of drugs used to treat mutations observed in target genes associated with certain cancer types.

TABLE 10
Exemplary drugs
a) Exemplary drugs

| Cancer | | |
|---|---|---|
| type | Drug | Target genes |
| Breast | abemaciclib (Verzenio) | CDK4, CDK6 |
| prostate | abiraterone acetate | CYP17A1 |
| | (Zytiga) | |
| leukemia | acalabrutinib (Calquence) | BTK |
| lymphoma | acalabrutinib (Calquence) | BTK |
| lung | adagrasib (Krazati) | KRAS G12C |
| Breast | ado-trastuzumab | Tubulin, Her2 |
| | emtansine (Kadcyla) | |
| lung | afatinib dimaleate | EGFR + EGFR exon 21 |
| | (Gilotrif) | L858R mutation + |
| | | EGFR-Ex19del + |
| | | HER2 + HER4 |
| lung | alectinib (Alecensa) | ALK, RET |
| leukemia | alemtuzumab (Campath) | CD52 |
| skin | alitretinoin (Panretin) | RARα + RARβ2 + |
| | | RARγ |
| soft tissue | alitretinoin (Panretin) | RARα + RARβ2 + |
| sarcoma | | RARγ |
| Breast | alpelisib (Pigray) | PI3Kα |
| lung | amivantamab-vmjw | EGFR + c-MET |
| | (Rybrevant) | |
| Breast | anastrozole (Arimidex) | Aromatase |
| prostate | apalutamide (Erleada) | AR |
| leukemia | asciminib hydrochloride | Bcr-Abl |
| | (Scemblix) | |
| bladder | atezolizumab (Tecentriq) | PDL1 |
| liver and | atezolizumab (Tecentriq) | PDL1 |
| bile duct | | |
| lung | atezolizumab (Tecentriq) | PDL1 |
| skin | atezolizumab (Tecentriq) | PDL1 |
| soft tissue | atezolizumab (Tecentriq) | PDL1 |
| sarcoma | | |
| Gastrointestinal | avapritinib (Ayvakit) | PDGFRα + c-Kit |
| systemic | avapritinib (Ayvakit) | PDGFRα + c-Kit |
| mastocytosis | | |
| bladder | avelumab (Bavencio) | PDL1 |
| endocrine | avelumab (Bavencio) | PDL1 |
| and | | |
| neuroendocrine | | |
| tumors | | |
| kidney | avelumab (Bavencio) | PDL1 |
| skin | avelumab (Bavencio) | PDL1 |
| lymphoma | axicabtagene ciloleucel | CD19 |
| | (Yescarta) | |
| kidney | axitinib (Inlyta) | VEGFR1 + VEGFR2 + |
| | | VEGFR3 |
| lymphoma | belinostat (Beleodaq) | HDAC |
| Brain | belzutifan (Welireg) | HIF-2A |
| kidney | belzutifan (Welireg) | HIF-2A |
| pancreatic | belzutifan (Welireg) | HIF-2A |
| ovarian | bevacizumab (Avastin) | VEGF-A |
| epithelial, | | |
| fallopian | | |
| tube, and | | |
| primary | | |
| peritoneal | | |
| Brain | bevacizumab (Avastin) | VEGF-A |
| Cervical | bevacizumab (Avastin) | VEGF-A |
| colorectal | bevacizumab (Avastin) | VEGF-A |
| kidney | bevacizumab (Avastin) | VEGF-A |
| liver and | bevacizumab (Avastin) | VEGF-A |
| bile duct | | |
| lung | bevacizumab (Avastin) | VEGF-A |
| lymphoma | bexarotene (Targretin) | RXRs |
| skin | binimetinib (Mektovi) | MEK1, MEK2 |
| leukemia | blinatumomab (Blincyto) | CD19, CD3 |
| lymphoma | bortezomib (Velcade) | Proteasome |
| multiple | bortezomib (Velcade) | Proteasome |
| myeloma | | |
| leukemia | bosutinib (Bosulif) | BCR-ABL, SRC |
| lymphoma | brentuximab vedotin | CD130, Tubulin |
| | (Adcetris) | |
| leukemia | brexucabtagene | CD19 |
| | autoleucel | |
| | (Tecartus) | |
| lymphoma | brexucabtagene | CD19 |
| | autoleucel | |
| | (Tecartus) | |
| lung | brigatinib (Alunbrig) | ALK, EGFR, FLT3, |
| | | IGF-1R, ROS1 |
| prostate | cabazitaxel (Jevtana) | Tubulin |
| kidney | cabozantinib-s-malate | AXL + RET + |
| | (Cabometyx) | ROS1 + TYRO3 + |
| | | Tie-2 + TrkB + |
| | | VEGFR1 + VEGFR2 + |
| | | VEGFR3 + c-Kit + |
| | | c-Met |
| liver and | cabozantinib-s-malate | AXL + RET + |
| bile duct | (Cabometyx) | ROS1 + TYRO3 + |
| | | Tie-2 + TrkB + |
| | | VEGFR1 + VEGFR2 + |
| | | VEGFR3 + c-Kit + |
| | | c-Met |
| thyroid | cabozantinib-s-malate | AXL + RET + |
| | (Cometriq) | ROS1 + TYRO3 + |
| | | Tie-2 + TrkB + |
| | | VEGFR1 + VEGFR2 + |
| | | VEGFR3 + c-Kit + |
| | | c-Met |
| lung | capmatinib hydrochloride | c-Met |
| | (Tabrecta) | |
| multiple | carfilzomib (Kyprolis) | protoasome |
| myeloma | | |
| lung | cemiplimab-rwlc | PD-1 |
| | (Libtayo) | |
| skin | cemiplimab-rwlc | PD-1 |
| | (Libtayo) | |
| lung | ceritinib (Zykadia) | ALK + IGF-1R + |
| | | INSR + ROS1 |
| colorectal | cetuximab (Erbitux) | EGFR |
| multiple | ciltacabtagene autoleucel | BCMA |
| myeloma | (Carvykti) | |
| skin | cobimetinib fumarate | MEK1, MEK2 |
| | (Cotellic) | |
| lymphoma | copanlisib hydrochloride | PI3Kα, PI3Kδ |
| | (Aliqopa) | |
| lung | crizotinib (Xalkori) | MTH1 |
| lymphoma | crizotinib (Xalkori) | MTH1 |
| Myofibroblastic | crizotinib (Xalkori) | MTH1 |
| Brain | dabrafenib (Tafinlar) | BRAF, CRAF |
| lung | dabrafenib mesylate | BRAF, CRAF |
| | (Tafinlar) | |
| skin | dabrafenib mesylate | BRAF, CRAF |
| | (Tafinlar) | |
| solid tumors | dabrafenib mesylate | BRAF, CRAF |
| anywhere in | (Tafinlar) | |
| the body | | |
| thyroid | dabrafenib mesylate | BRAF, CRAF |
| | (Tafinlar) | |
| lung | dacomitinib (Vizimpro) | EGFR + EGFR exon 21 |
| | | L858R mutation + |
| | | EGFR-Ex19del + |
| | | HER2 + HER4 |
| multiple | daratumumab (Darzalex) | CD38 + Hyaluronic acid |
| myeloma | | |
| multiple | daratumumab and | CD38 + Hyaluronic acid |
| myeloma | hyaluronidase-fihj | |
| | (Darzalex Faspro) | |
| prostate | darolutamide (Nubeqa) | AR |
| leukemia | dasatinib (Sprycel) | Bcr-Abl + EphA2 + |
| | | FYN + LCK + |
| | | PDGFRβ + Protein- |
| | | tyrosine kinases + |
| | | SRC + YES1 + c-Kit |
| neuroblastoma | dinutuximab (Unituxin) | GD2 |
| endometrial | dostarlimab-gxly | PD-1 |
| | (Jemperli) | |
| solid tumors | dostarlimab-gxly | PD-1 |
| anywhere in | (Jemperli) | |
| the body | | |
| liver and | durvalumab (Imfinzi) | PDL1 |
| bile duct | | |
| lung | durvalumab (Imfinzi) | PDL1 |
| leukemia | duvelisib (Copiktra) | PI3Kγ + PI3Kδ |
| Breast | elacestrant | Erα/ESR1 |
| | dihydrochloride | |
| | (Orserdu) | |
| multiple | elotuzumab (Empliciti) | SLAMF7 |
| myeloma | | |
| leukemia | enasidenib mesylate | IDH2 |
| | (Idhifa) | |
| colorectal | encorafenib (Braftovi) | BRAF, BRAF V600E |
| skin | encorafenib (Braftovi) | BRAF V600E, V600K |
| | | mutation |
| bladder | enfortumab vedotin-ejfv | Nectin-4, Tubulins |
| | (Padcev) | |
| lung | entrectinib (Rozlytrek) | ALK + ROS1 + |
| | | TRKA + TrkB + |
| | | TrkC |
| solid tumors | entrectinib (Rozlytrek) | ALK + ROS1 + |
| anywhere in | | TRKA + TrkB + |
| the body | | TrkC |
| prostate | enzalutamide (Xtandi) | AR |
| bladder | erdafitinib (Balversa) | FGFR1, FGFR2, FGFR3, |
| | | FGFR4 |
| lung | erlotinib hydrochloride | EGFR antagonists, |
| | (Tarceva) | EGFR exon 21 |
| | | L858R mutation |
| | | inhibitors, |
| | | EGFR-Ex19del |
| | | inhibitors |
| pancreatic | erlotinib hydrochloride | EGFR + EGFR exon 21 |
| | (Tarceva) | L858R mutation + |
| | | EGFR-Ex19del |
| Breast | everolimus (Afinitor) | mTORC1, mTORC2 |
| kidney | everolimus (Afinitor) | mTORC1, mTORC2 |
| pancreatic | everolimus (Afinitor) | mTORC1, mTORC2 |
| Breast | exemestane (Aromasin) | Aromatase |
| Breast | fam-trastuzumab | HER2, TOP1 |
| | deruxtecan-nxki | |
| | (Enhertu) | |
| Gastric | fam-trastuzumab | HER2, TOP1 |
| | deruxtecan-nxki | |
| | (Enhertu) | |
| lung | fam-trastuzumab | HER2, TOP1 |
| | deruxtecan-nxki | |
| | (Enhertu) | |
| myelodysplastic | fedratinib hydrochloride | JAK2, FLT3 |
| and | (Inrebic) | |
| myelopro- | | |
| liferative | | |
| disorders | | |
| Breast | fulvestrant (Faslodex) | ER |
| liver and | futibatinib (Lytgobi) | FGFRs |
| bile duct | | |
| lung | gefitinib (Iressa) | EGFR + EGFR exon 21 |
| | | L858R mutation + |
| | | EGFR-Ex19del |
| leukemia | gemtuzumab ozogamicin | CD33, DNA |
| | (MYLOTARG) | |
| leukemia | gilteritinib fumarate | AXL + FLT3 |
| | (Xospata) | |
| leukemia | glasdegib | SMO |
| | maleate (Daurismo) | |
| lymphoma | ibritumomab tiuxetan | CD20 |
| | (Zevalin) | |
| leukemia | ibrutinib (Imbruvica) | BTK |
| lymphoma | ibrutinib (Imbruvica) | BTK |
| multiple | idecabtagene vicleucel | BCMA |
| myeloma | (Abecma) | |
| leukemia | idelalisib (Zydelig) | PI3Kδ |
| dermatofibr | imatinib mesylate | BCR-ABL, PDGFR, |
| osarcoma | (Gleevec) | C-Kit |
| protuberans | | |
| Gastrointestinal | imatinib mesylate | BCR-ABL, PDGFR, |
| | (Gleevec) | C-Kit |
| leukemia | imatinib mesylate | BCR-ABL, PDGFR, |
| | (Gleevec) | C-Kit |
| myelodysplastic | imatinib mesylate | BCR-ABL, PDGFR, |
| and | (Gleevec) | C-Kit |
| myelopro- | | |
| liferative | | |
| disorders | | |
| systemic | imatinib mesylate | BCR-ABL, PDGFR, |
| mastocytosis | (Gleevec) | C-Kit |
| liver and | infigratinib phosphate | FGFR1 + FGFR2 + |
| bile duct | (Truseltiq) | FGFR3 + FGFR4 |
| leukemia | inotuzumab ozogamicin | CD22 + DNA |
| | (Besponsa) | |
| endocrine | iobenguane I 131 | NET |
| and | (Azedra) | |
| neuroendocrine | | |
| tumors | | |
| colorectal | ipilimumab (Yervoy) | CTLA4 |
| esophageal | ipilimumab (Yervoy) | CTLA4 |
| kidney | ipilimumab (Yervoy) | CTLA4 |
| liver and | ipilimumab (Yervoy) | CTLA4 |
| bile duct | | |
| lung | ipilimumab (Yervoy) | CTLA4 |
| malignant | ipilimumab (Yervoy) | CTLA4 |
| mesothelioma | | |
| skin | ipilimumab (Yervoy) | CTLA4 |
| multiple | isatuximab-irfc (Sarclisa) | CD38 |
| myeloma | | |
| leukemia | ivosidenib (Tibsovo) | IDH1 |
| multiple | ixazomib citrate | proteasome |
| myeloma | (Ninlaro) | |
| endocrine | lanreotide acetate | SSTR |
| and | (Somatuline Depot) | |
| neuroendocrine | | |
| tumors | | |
| Breast | lapatinib ditosylate | EGFR |
| | (Tykerb) | |
| solid tumors | larotrectinib sulfate | TRKA + TrkB + |
| anywhere in | (Vitrakvi) | TrkC |
| the body | | |
| endometrial | lenvatinib mesylate | FGFR1 + FGFR2 + |
| | (Lenvima) | FGFR3 + FGFR4 + |
| | | PDGFRα + RET + |
| | | VEGFR1 + VEGFR2 + |
| | | VEGFR3 + c-Kit |
| kidney | lenvatinib mesylate | FGFR1 + FGFR2 + |
| | (Lenvima) | FGFR3 + FGFR4 + |
| | | PDGFRα + RET + |
| | | VEGFR1 + VEGFR2 + |
| | | VEGFR3 + c-Kit |
| liver and | lenvatinib mesylate | FGFR1 + FGFR2 + |
| bile duct | (Lenvima) | FGFR3 + FGFR4 + |
| | | PDGFRα + RET + |
| | | VEGFR1 + VEGFR2 + |
| | | VEGFR3 + c-Kit |
| thyroid | lenvatinib mesylate | FGFR1 + FGFR2 + |
| | (Lenvima) | FGFR3 + FGFR4 + |
| | | PDGFRα + RET + |
| | | VEGFR1 + VEGFR2 + |
| | | VEGFR3 + c-Kit |
| Breast | letrozole | CDK4/ER |
| | (Femara)/Ribociclib | |
| | Succinate | |
| lymphoma | lisocabtagene maraleucel | CD19 |
| | (Breyanzi) | |
| lymphoma | loncastuximab tesirine- | CD19 + DNA |
| | lpyl (Zynlonta) | |
| lung | lorlatinib (Lorbrena) | ALK + ROS1 |
| prostate | lutetium Lu 177 | PSMA |
| | vipivotide tetraxetan | |
| | (Pluvicto) | |
| endocrine | lutetium Lu 177-dotatate | SSTR2 |
| and | (Lutathera) | |
| neuroendocrine | | |
| tumors | | |
| Breast | margetuximab-cmkb | HER2 |
| | (Margenza) | |
| leukemia | midostaurin (Rydapt) | FLT3 + PDGFR + |
| | | PKC + Syk + |
| | | VEGFR2 + c-Kit |
| systemic | midostaurin (Rydapt) | FLT3 + PDGFR + |
| mastocytosis | | PKC + Syk + |
| | | VEGFR2 + c-Kit |
| ovarian | mirvetuximab | FOLR1 + Tubulin |
| epithelial, | soravtansine-gynx | |
| fallopian | (Elahere) | |
| tube, and | | |
| primary | | |
| peritoneal | | |
| lung | mobocertinib | EGFR exon 20 + |
| | succinate | HER2 exon 20 |
| | (Exkivity) | |
| lymphoma | mogamulizumab-kpkc | CCR4 |
| | (Poteligeo) | |
| lymphoma | mosunetuzumab-axgb | CD20, CD3 |
| | (Lunsumio) | |
| leukemia | moxetumomab | CD22 |
| | pasudotox- | |
| | tdfk (Lumoxiti) | |
| neuroblastoma | naxitamab-gqgk | GD2 |
| | (Danyelza) | |
| lung | necitumumab (Portrazza) | EGFR |
| Breast | neratinib maleate | EGFR, HER2, HER4 |
| | (Nerlynx) | |
| leukemia | nilotinib (Tasigna) | Bcr-Abl + CSF-1R + |
| | | DDR1 + PDGFR + |
| | | c-Kit |
| ovarian | niraparib tosylate | PARP1 + PARP2 |
| epithelial, | monohydrate (Zejula) | |
| fallopian | | |
| tube, and | | |
| primary | | |
| peritoneal | | |
| bladder | nivolumab (Opdivo) | PD-1 |
| colorectal | nivolumab (Opdivo) | PD-1 |
| esophageal | nivolumab (Opdivo) | PD-1 |
| kidney | nivolumab (Opdivo) | PD-1 |
| liver and | nivolumab (Opdivo) | PD-1 |
| bile duct | | |
| lung | nivolumab (Opdivo) | PD-1 |
| lymphoma | nivolumab (Opdivo) | PD-1 |
| malignant | nivolumab (Opdivo) | PD-1 |
| mesothelioma | | |
| skin | nivolumab (Opdivo) | PD-1 |
| stomach | nivolumab (Opdivo) | PD-1 |
| (gastric) | | |
| skin | nivolumab and | LAG3 + PD-1 |
| | relatlimab- | |
| | rmbw (Opdualag) | |
| leukemia | obinutuzumab (Gazyva) | CD20 |
| lymphoma | obinutuzumab (Gazyva) | CD20 |
| leukemia | ofatumumab (Arzerra) | CD20 |
| ovarian | olaparib (Lynparza) | PARP1, PARP2, PARP3 |
| epithelial, | | |
| fallopian | | |
| tube, and | | |
| primary | | |
| peritoneal | | |
| Breast | olaparib (Lynparza) | PARP1, PARP2, PARP3 |
| pancreatic | olaparib (Lynparza) | PARP1, PARP2, PARP3 |
| prostate | olaparib (Lynparza) | PARP1, PARP2, PARP3 |
| leukemia | olutasidenib (Rezlidhia) | IDH1 |
| lung | osimertinib mesylate | EGFR + EGFR T790M + |
| | (Tagrisso) | EGFR exon 21 L858R |
| | | mutation + EGFR- |
| | | Ex19del |
| myelodysplastic | pacritinib citrate (Vonjo) | CSF-1R + FLT3 + |
| and | | IRAK1 + JAK2 |
| myelopro- | | |
| liferative | | |
| disorders | | |
| Breast | palbociclib (Ibrance) | CDK4, CDK6 |
| colorectal | panitumumab (Vectibix) | EGFR |
| kidney | pazopanib | FGFR1 + FGFR3 + |
| | hydrochloride (Votrient) | Flt3L + ITK + |
| | | LCK + PDGFRα + |
| | | PDGFRβ + VEGFR1 + |
| | | VEGFR2 + VEGFR3 + |
| | | c-Kit |
| soft tissue | pazopanib hydrochloride | FGFR1 + FGFR3 + |
| sarcoma | (Votrient) | Flt3L + ITK + LCK + |
| | | PDGFRα + PDGFRβ + |
| | | VEGFR1 + VEGFR2 + |
| | | VEGFR3 + c-Kit |
| Breast | pembrolizumab | PD-1 |
| | (Keytruda) | |
| Cervical | pembrolizumab | PD-1 |
| | (Keytruda) | |
| colorectal | pembrolizumab | PD-1 |
| | (Keytruda) | |
| endometrial | pembrolizumab | PD-1 |
| | (Keytruda) | |
| esophageal | pembrolizumab | PD-1 |
| | (Keytruda) | |
| kidney | pembrolizumab | PD-1 |
| | (Keytruda) | |
| liver and | pembrolizumab | PD-1 |
| bile duct | (Keytruda) | |
| lung | pembrolizumab | PD-1 |
| | (Keytruda) | |
| lymphoma | pembrolizumab | PD-1 |
| | (Keytruda) | |
| skin | pembrolizumab | PD-1 |
| | (Keytruda) | |
| solid tumors | pembrolizumab | PD-1 |
| anywhere in | (Keytruda) | |
| the body | | |
| stomach | pembrolizumab | PD-1 |
| (gastric) | (Keytruda) | |
| leukemia | pemigatinib (Pemazyre) | FGFR1, FGFR2, FGFR3, |
| | | FGFR4 |
| liver and | pemigatinib (Pemazyre) | FGFR1, FGFR2, FGFR3, |
| bile duct | | FGFR4 |
| lymphoma | pemigatinib (Pemazyre) | FGFR1, FGFR2, FGFR3, |
| | | FGFR4 |
| myelodysplastic | pemigatinib (Pemazyre) | FGFR1, FGFR2, FGFR3, |
| and | | FGFR4 |
| myelopro- | | |
| liferative | | |
| disorders | | |
| Breast | pertuzumab (Perjeta) | HER2 |
| Breast | pertuzumab, | HER2, Hyaluronic acid |
| | trastuzumab, and | |
| | hyaluronidase-zzxf | |
| | (Phesgo) | |
| giant cell | pexidartinib | CSF-1R + FLT3 + |
| tumor | hydrochloride | c-Kit |
| | (Turalio) | |
| lymphoma | pirtobrutinib (Jaypirca) | BTK C481S |
| lymphoma | polatuzumab vedotin-piiq | CD79B + Tubulin |
| | (Polivy) | |
| leukemia | ponatinib hydrochloride | Bcr-Abl + Protein- |
| | (Iclusig) | tyrosine kinases |
| lymphoma | pralatrexate (Folotyn) | Bcr-Abl + Protein- |
| | | tyrosine kinases |
| lung | pralsetinib (Gavreto) | RET |
| thyroid | pralsetinib (Gavreto) | RET |
| prostate | radium 223 dichloride | DNA |
| | (Xofigo) | |
| colorectal | ramucirumab (Cyramza) | VEGFR2 |
| Gastric | ramucirumab (Cyramza) | VEGFR2 |
| liver and | ramucirumab (Cyramza) | VEGFR2 |
| bile duct | | |
| lung | ramucirumab (Cyramza) | VEGFR2 |
| stomach | ramucirumab (Cyramza) | VEGFR2 |
| (gastric) | | |
| colorectal | regorafenib (Stivarga) | Abl family, BRAF, BRAF |
| | | V600E, CRAF, CSF-1R, |
| | | DDR2, EphA2, FGFR1, |
| | | FGFR2, FRK, MAPK11, |
| | | PDGFRa, PDGFRb, RET, |
| | | TRKA, Tie-2, VEGFR1, |
| | | VEGFR2, VEGFR3, c-kit |
| Gastrointestinal | regorafenib (Stivarga) | Abl family, BRAF, BRAF |
| | | V600E, CRAF, CSF-1R, |
| | | DDR2, EphA2, FGFR1, |
| | | FGFR2, FRK, MAPK11, |
| | | PDGFRa, PDGFRb, RET, |
| | | TRKA, Tie-2, VEGFR1, |
| | | VEGFR2, VEGFR3, c-kit |
| liver and | regorafenib (Stivarga) | Abl family, BRAF, BRAF |
| bile duct | | V600E, CRAF, CSF-1R, |
| | | DDR2, EphA2, FGFR1, |
| | | FGFR2, FRK, MAPK11, |
| | | PDGFRa, PDGFRb, RET, |
| | | TRKA, Tie-2, VEGFR1, |
| | | VEGFR2, VEGFR3, c-kit |
| skin | retifanlimab-dlwr | PD-1 |
| | (Zynyz) | |
| Breast | ribociclib (Kisqali) | CDK4, CDK6 |
| gastrointestinal | ripretinib (Qinlock) | EGFR + PDGFRα + c-Kit |
| leukemia | rituximab (Rituxan) | CD20 + Hyaluronic acid |
| lymphoma | rituximab (Rituxan) | CD20 + Hyaluronic acid |
| leukemia | rituximab and | CD20 + Hyaluronic acid |
| | hyaluronidase human | |
| | (Rituxan Hycela) | |
| lymphoma | rituximab and | CD20 + Hyaluronic acid |
| | hyaluronidase human | |
| | (Rituxan Hycela) | |
| lymphoma | romidepsin (Istodax) | HDAC |
| ovarian | rucaparib | PARP1 + PARP2 + PARP3 |
| epithelial, | camsylate (Rubraca) | |
| fallopian | | |
| tube, and | | |
| primary | | |
| peritoneal | | |
| prostate | rucaparib camsylate | PARP1 + PARP2 + PARP3 |
| | (Rubraca) | |
| myelodysplastic | ruxolitinib phosphate | JAK1 + JAK2 |
| and | (Jakafi) | |
| myelopro- | | |
| liferative | | |
| disorders | | |
| Breast | sacituzumab govitecan- | TOP1 |
| | hziy (Trodelvy) | |
| lymphoma | selinexor (Xpovio) | XPO1 |
| multiple | selinexor (Xpovio) | XPO1 |
| myeloma | | |
| lung | selpercatinib (Retevmo) | RET |
| solid tumors | selpercatinib (Retevmo) | RET |
| anywhere in | | |
| the body | | |
| thyroid | selpercatinib (Retevmo) | RET |
| plexiform | selumetinib sulfate | MEK1 + MEK2 |
| neurofibroma | (Koselugo) | |
| soft tissue | sirolimus protein-bound | MUT + mTOR |
| sarcoma | particles (Fyarro) | |
| skin | sonidegib (Odomzo) | SMO |
| kidney | sorafenib tosylate | BRAF inhibitors, CRAF |
| | (Nexavar) | inhibitors, FLT3 + |
| | | PDGFRβ + RET + |
| | | VEGFR1 + VEGFR2 + |
| | | VEGFR3 + c-Kit |
| liver and | sorafenib tosylate | BRAF + CRAF + FLT3 + |
| bile duct | (Nexavar) | PDGFRβ + RET + |
| | | VEGFR1 + VEGFR2 + |
| | | VEGFR3 + c-Kit |
| thyroid | sorafenib tosylate | BRAF + CRAF + FLT3 + |
| | (Nexavar) | PDGFRβ + RET + |
| | | VEGFR1 + VEGFR2 + |
| | | VEGFR3 + c-Kit |
| lung | sotorasib (Lumakras) | KRAS G12C |
| Gastrointestinal | sunitinib malate (Sutent) | PDGFR + RTK + VEGFR |
| kidney | sunitinib malate (Sutent) | PDGFR + RTK + VEGFR |
| pancreatic | sunitinib malate (Sutent) | PDGFR + RTK + VEGFR |
| lymphoma | tafasitamab-cxix | CD19 |
| | (Monjuvi) | |
| leukemia | tagraxofusp-erzs | CD123 |
| | (Elzonris) | |
| Breast | talazoparib tosylate | PARP1, PARP2 |
| | (Talzenna) | |
| Breast | tamoxifen citrate | ER |
| | (Soltamox) | |
| lymphoma | tazemetostat | EZH2 |
| | hydrobromide (Tazverik) | |
| soft tissue | tazemetostat | EZH2 |
| sarcoma | hydrobromide (Tazverik) | |
| skin | tebentafusp-tebn | CD3 + gp100 |
| | (Kimmtrak) | |
| multiple | teclistamab-cqyv | CD3 + gp100 |
| myeloma | (Tecvayli) | |
| kidney | temsirolimus (Torisel) | CD3 + gp100 |
| lung | tepotinib hydrochloride | MET |
| | (Tepmetko) | |
| leukemia | tisagenlecleucel | CD19 |
| | (Kymriah) | |
| lymphoma | tisagenlecleucel | CD19 |
| | (Kymriah) | |
| Cervical | tisotumab vedotin-tftv | Tubulin, tissue factor |
| | (Tivdak) | |
| kidney | tivozanib hydrochloride | (VEGFR)-1, VEGFR-2 and |
| | (Fotivda) | VEGFR-3, c-kit, and |
| | | PDGFRβ |
| Breast | toremifene (Fareston) | ER |
| Brain | trametinib (Mekinist) | MEK1 and MEK2 |
| lung | trametinib dimethyl | MEK1 and MEK2 |
| | sulfoxide (Mekinist) | |
| skin | trametinib dimethyl | MEK1 and MEK2 |
| | sulfoxide (Mekinist) | |
| solid tumors | trametinib dimethyl | MEK1 and MEK2 |
| anywhere in | sulfoxide (Mekinist) | |
| the body | | |
| thyroid | trametinib dimethyl | MEK1 and MEK2 |
| | sulfoxide (Mekinist) | |
| Breast | trastuzumab (Herceptin) | HER2 |
| esophageal | trastuzumab (Herceptin) | HER2 |
| stomach | trastuzumab (Herceptin) | HER2 |
| (gastric) | | |
| liver and | tremelimumab-actl | CTLA-4, CD80, and CD86 |
| bile duct | (Imjudo) | |
| lung | tremelimumab-actl | CTLA-4, CD80, and CD86 |
| | (Imjudo) | |
| Breast | tucatinib (Tukysa) | HER2 |
| colorectal | tucatinib (Tukysa) | HER2 |
| thyroid | vandetanib (Caprelsa) | VEGFR-2, EGFR, RET |
| skin | vemurafenib (Zelboraf) | BRAF, BRAF V600E, |
| | | CRAF, ARAF, SRMS, |
| | | ACK1, MAP4K5, FGR |
| leukemia | venetoclax (Venclexta) | BCL-2 |
| lymphoma | venetoclax (Venclexta) | BCL-2 |
| lymphoma | vorinostat (Zolinza) | HDAC1, HDAC2 and |
| | | HDAC3 (Class I) |
| | | and HDAC6 |
| | | (Class II) |
| leukemia | zanubrutinib (Brukinsa) | BTK |
| lymphoma | zanubrutinib (Brukinsa) | BTK |
| colorectal | ziv-aflibercept (Zaltrap) | PGF, VEGF-A |

In some embodiments, the methods described herein can be used to treat patients by (i) detecting one or more somatic mutations and/or cancer-related epigenetic signatures in the one or more target genes listed in Table 10; and (ii) administering the corresponding one or more drugs listed in Table 10. In some embodiments, these therapies may be used alone or in combination with other therapies to treat a disease.

In certain embodiments, the status of a nucleic acid variant from a sample from a subject as being of somatic or germline origin may be compared with a database of comparator results from a reference population to identify customized or targeted therapies for that subject.

Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA (e.g., cfDNA) isolated from a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject.

Thus, in some embodiments, quantities of each of one or more of a particular genetic and/or epigenetic signature (e.g, quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g, copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, genetic and/or epigenetic signatures, and/or cell types, are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

Therapeutic options for treating specific genetic-based diseases, disorders, or conditions, other than cancer, are generally well-known to those of ordinary skill in the art and will be apparent given the particular disease, disorder, or condition under consideration.

### III. Kits

The disclosure also provides kits for practice of (e.g., for use in) any of the methods described herein. An exemplary kit includes oligonucleotide probes for targeting and capturing a sequencing panel as discussed herein. Kits can also include packaging, leaflets, CDs or the like providing instructions for practice of the claimed methods. Also provided are kits, e.g., comprising the compositions as described herein. The kits can be useful in, or for use in, performing the methods as described herein. In some embodiments, the kit comprises target-specific probes that specifically bind to epigenetic and/or sequence-variable target region sets, wherein the target-specific probes of at least one epigenetic target region set bind to target regions that are differentially methylated in different immune cell types. In some such embodiments, the target-specific probes comprise a capture moiety. In some embodiments, the kit comprises a solid support linked to a binding partner of the capture moiety.

In some embodiments, a kit comprises an agent that recognizes methyl cytosine in DNA. In some such embodiments, the agent is an antibody or a methyl binding protein or methyl binding domain.

In some embodiments, a kit comprises reagents for a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity. The procedure that affects a first nucleobase of the DNA differently from a second nucleobase of the DNA may be any of the procedures described elsewhere herein.

In some embodiments, the kit comprises one or more conversion reagents. The conversion reagents may comprise reagents for any combination of steps described herein, including but not limited to in the numbered embodiments above and in any one of the workflows shown in the figures. In some embodiments, the kit comprises a TET2 enzyme comprising a T1372S mutation and a substituted borane reducing agent. The enzyme and reducing agent may be according to any of the embodiments thereof described elsewhere herein.

In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSF1R, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPMI, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the can be found throughout and are applicable to the kit.

### Additional Embodiments

The following additional embodiments are provided herein.

Embodiment A1 is a method for determining reduced efficacy of a genome editing drug, comprising:
(a) collecting a sample from a subject, wherein the subject has undergone administration of the genome editing drug to treat a disease;
(b) determining a first parameter comprising quantitative measures of levels of one or more proteins associated with the disease by performing a proteomic assay on molecules obtained or derived from a first portion of the sample;
(c) determining a second parameter comprising quantitative measures of editing of one or more predetermined variants associated with the disease by sequencing a sequencing library comprising molecules obtained or derived from a second portion of the sample; and
(d) determining whether the first and the second parameters are within a predetermined therapeutic threshold, wherein the first and second parameters below the predetermined therapeutic threshold indicate an additional dose of the genome editing drug should be administered to the subject.

Embodiment A2 is the method of embodiment A1, wherein the method further comprises determining adverse side effects of the genome editing drug by:
(e) sequencing a panel to generate sequencing reads from molecules obtained or derived from a third portion of the sample; and
(f) detecting from the sequencing reads the presence or absence of one or more genetic, epigenetic, and/or fragmentomic markers, wherein the presence or absence of one or more of the markers is indicative of the adverse side effect induced by the genome editing drug.

Embodiment A3 is the method of any one of embodiments A1-A2, wherein the genome editing drug comprises a CRISPR-Cas system that edits the one or more predetermined variants associated with the disease.

Embodiment A4 is the method of any one of embodiments A1-A3, wherein the genome editing drug further comprises one or more guide RNAs (sgRNAs) that direct a Cas protein to the one or more predetermined variants associated with the disease.

Embodiment A5 is the method of embodiment A4, wherein the Cas proteins comprises Cas9, dCas9, or nCas9.

Embodiment A6 is the method of embodiment A3, wherein the CRISPR-Cas system introduces a gene knockout, gene insertion, a wildtype allele, or an inactivating mutation.

Embodiment A7 is the method of any one of embodiments A1-A6, wherein the sample is a bodily sample comprising blood, plasma, serum, or urine sample.

Embodiment A8 is the method of any one of embodiments A1-A7, wherein the proteomic assay comprises an immunoassay, ELISA, HPLC, mass spectrometry, ProtSeq, IHC or immunofluorescence.

Embodiment A9 is the method of embodiment A1, wherein the genome editing drug is administered in vivo or ex vivo.

Embodiment A10 is the method of embodiment A9, wherein the ex vivo genome editing is done in hematopoietic stem cells.

Embodiment A11 is the method of embodiment A10, wherein the hematopoietic stem cells comprise a cell-based therapy.

Embodiment A12 is the method of embodiment A11, wherein the therapy comprises an immunotherapy.

Embodiment A13 is the method of any one of embodiments A1-A12, where in the disease is amyloidosis transthyretin-related (ATTR).

Embodiment A14 is the method of any one of embodiments A1-A13, wherein the one or more variants are within the TTR gene sequence.

Embodiment A15 is the method of any one of embodiments A1-A14, wherein the disease is a hemoglobinopathy.

Embodiment A16 is the method of embodiment A15, wherein the hemoglobinopathy is beta-thalassemia or sickle cell disease.

Embodiment A17 is the method of any one of embodiments A1-A16, wherein the adverse side effects is cancer.

Embodiment A18 is the method of embodiment A2, wherein at least a subset of the markers in (b) are specific to the genome editing drug.

Embodiment A19 is the method of embodiment A2, wherein at least a subset of the markers in (b) are specific to the subject based on the subject's genomic profile.

Embodiment A20 is the method of embodiment A2, wherein the markers in (b) further comprise one or more of a SNV, indels, structural variants, methylation, histone acetylation, TFBS, chromatin positioning, or nucleosome positioning.

Embodiment A21 is a method of screening for a disease induced by a genome editing drug, comprising:
(a) collecting a sample from a subject, wherein the subject has undergone administration of the genome editing drug;
(b) sequencing a sequencing panel comprising nucleic acid molecules obtained or derived from the sample to generate sequencing reads; and
(c) detecting from the sequencing reads the presence or absence of one or more genetic, epigenetic, and/or fragmentomic markers, wherein the presence or absence of one or more of the markers is indicative of the disease induced by the genome editing drug.

Embodiment A22 is the method of embodiment A21, wherein the nucleic acid molecules are DNA.

Embodiment A23 is the method of embodiment A22, wherein the DNA is cell-free DNA.

Embodiment A24 is the method of any one of embodiments A21-A23, further comprising generating copies of the cell-free DNA molecules prior to sequencing.

Embodiment A25 is the method of any one of embodiments A21-A24, further comprising attaching one or more adapters comprising barcodes to the nucleic acid molecules prior to sequencing.

Embodiment A26 is the method of embodiment A25, wherein the adapters are randomly attached to both ends of the nucleic acid molecules.

Embodiment A27 is the method of any one of embodiments A21-A26, wherein the nucleic acid molecules are uniquely barcoded.

Embodiment A28 is the method of any one of embodiments A21-A27, wherein the nucleic acid molecules are non-uniquely barcoded.

Embodiment A29 is a sequencing panel, comprising:
a plurality of target regions,
wherein the plurality of target regions comprise a plurality of loci associated with a genetic disease in a subject,
wherein the plurality of loci comprise one or more nucleotide targets for a genome editing drug.

Embodiment A30 is the sequencing panel of embodiment A29, wherein the sequencing panel is specific to the subject based on the subject's genomic profile.

Embodiment A31 is the sequencing panel of any one of embodiments A29-A30, wherein the panel comprises a plurality of nucleic acid sequences specific to a genome editing drug.

Embodiment A32 is a method for determining therapeutic efficacy and adverse side effects of a genome editing drug, comprising:
(a) collecting a sample from a subject, wherein the subject has undergone administration of the genome editing drug to treat a disease;
(b) determining a first parameter comprising quantitative measures of levels of one or more proteins associated with the disease by performing a proteomic assay on molecules obtained or derived from a first portion of the sample;
(c) determining a second parameter comprising quantitative measures of editing of one or more predetermined variants associated with the disease by sequencing a sequencing library comprising molecules obtained or derived from a second portion of the sample; and
(d) determining whether the first and the second parameters are within a predetermined therapeutic threshold, wherein the first and second parameters below the predetermined therapeutic threshold indicate an additional dose of the genome editing drug should be administered to the subject;
(e) sequencing a panel to generate sequencing reads from molecules obtained or derived from a third portion of the sample; and
(f) detecting from the sequencing reads the presence or absence of one or more genetic, epigenetic, and/or fragmentomic markers, wherein the presence or absence of one or more of the markers is indicative of the adverse side effect induced by the genome editing drug.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

1.-91. (canceled)

92. A method of preparing a panel of hybridization probes, comprising:
(a) sequencing nucleic acid molecules obtained or derived from a sample from a subject that has undergone administration of a genome editing drug to generate sequencing reads;
(b) identifying a plurality of genomic locations with alterations from among a plurality of the sequencing reads; and
(c) preparing the panel of hybridization probes based on information obtained from the plurality of sequencing reads, wherein the panel comprises hybridization probes specific for at least a portion of the plurality of genomic locations with alterations.

93. The method of claim 92, wherein the hybridization probes are labeled with a binding partner, optionally wherein the binding partner is biotin.

94. A method of preparing a sequencing library, comprising:
(a) sequencing nucleic acid molecules obtained or derived from a first sample from a subject that has undergone administration of a genome editing drug to generate sequencing reads;
(b) identifying a plurality of genomic locations with alterations from among a plurality of the sequencing reads;
(c) enriching a second sample from the subject for a plurality of target regions, wherein the target regions comprise at least a portion of the genomic locations identified in (b), thereby providing enriched target regions; and
(d) preparing a sequencing library comprising at least a portion of the enriched target regions.

95. The method claim 94, wherein enriching the second sample comprises targeted amplification of the plurality of target regions.

96. The method of claim 94, wherein enriching the second sample comprises capturing the plurality of target regions with a plurality of hybridization probes.

97. The method of claim 94, wherein preparing the sequencing library comprises adding adapters to at least a portion of the enriched target regions.

98. The method of claim 97, wherein the adapters comprise barcodes.

99. The method of claim 94, wherein preparing the sequencing library comprises amplifying the enriched target regions.

100. The method of claim 94, wherein the alterations comprise on-target genetic alterations.

101. The method of claim 94, wherein the alterations comprise off-target genetic alterations.

102. The method of claim 9, wherein the alterations comprise epigenetic alterations.

103. (canceled)

104. The method of claim 94, wherein the alterations are relative to a wild-type reference genome.

105. The method of claim 94, wherein the alterations are relative to the genome of the subject before administration of the genome editing drug.

106. The method of claim 96, wherein the hybridization probes are labeled with a binding partner.

107. The method of claim 106, wherein the binding partner is biotin.
